SEQUENCE LISTING (24 sequences)

SEQ ID NO: 1 - DNA, 3353 bp, Homo sapiens
  source 1..3353: mol_type = genomic DNA
ctctgtggagtctagctgccagggtcgcggcagctgcggggagagatgactggggagcgacccagcacggcgctcccggacagacgctgggggccgcggattctgggcttctggggaggctgtagggtttgggtgtttgccgcgatcttcctgctgctgtctctggcagcctcctggtccaaggctgagaacgacttcggtctggtgcagccgctggtgaccatggagcaactgctgtgggtgagcgggagacagatcggctcagtggacaccttccgcatcccgctcatcacagccactccgcggggcactcttctcgcctttgctgaggcgaggaaaatgtcctcatccgatgagggggccaagttcatcgccctgcggaggtccatggaccagggcagcacatggtctcctacagcgttcattgtcaatgatggggatgtccccgatgggctgaaccttggggcagtagtgagcgatgttgagacaggagtagtatttcttttctactccctttgtgctcacaaggccggctgccaggtggcctctaccatgttggtatggagcaaggatgatggtgtttcctggagcacaccccggaatctctccctggatattggcactgaagtgtttgcccctggaccgggctctggtattcagaaacagcgggagccacggaagggccgcctcatcgtgtgtggccatgggacgctggagcgggacggagtcttctgtctcctcagcgatgatcatggtgcctcctggcgctacggaagtggggtcagcggcatcccctacggtcagcccaagcaggaaaatgatttcaatcctgatgaatgccagccctatgagctcccagatggctcagtcgtcatcaatgcccgaaaccagaacaactaccactgccactgccgaattgtcctccgcagctatgatgcctgtgatacactaaggccccgtgatgtgaccttcgaccctgagctcgtggaccctgtggtagctgcaggagctgtagtcaccagctccggcattgtcttcttctccaacccagcacatccagagttccgagtgaacctgaccctgcgatggagcttcagcaatggtacctcatggcggaaagagacagtccagctatggccaggccccagtggctattcatccctggcaaccctggagggcagcatggatggagaggagcaggccccccagctctacgtcctgtatgagaaaggccggaaccactacacagagagcatctccgtggccaaaatcagtgtctatgggacactctgagctgtgccactgccacaggggtattctgccttcaggactctgccttcaggaacacgggtctgtagagggtctgctggagacgcctgaaagacagttccatcttcctttagactccagccttggcaaaatcaccttccctttaccagggaaatcacttcctttaggactgaaagctaggcgtcctctcccacaaaaaagtcctgccctcatctgagaatactgtctttccatatggctaagtgtggccccaccaccctctctgccctcccgggacattgattggtcctgtcttgggcaggtctagtgagctgtagaattgaatcaatgtgaactcagggaactggggaaggctgagcctcctctttggtgttgcggtaagataaccgacagggctggtgaaagtccccagatggcaggatatttggtttcagagtaaggactaggtgcaccaccatgactgactatcaatcaaaatgtttgtaacttaaaatttttaatgaaggataatgaatatttgtagagtctctatggttctgtcaatgcacatcttcgtgtctgttttcctcatgtatccttgtgagcctgggtgagttctggggagagacctgatgtgcgtactgcctgtgaaaatctgactttggcaaatcaaatcctcttttccttttgacatgccctctttttttgttgttgctttttttgagacagggctcgctctgtcacccaggctggagtgcagttgcacaatcacggctcactgaagcctcaacctcctgggctcaagtgatcctcacgtctcagcctccggagtagttgggactacaggtcagtgacaccatgcctggttaatttttttaatttttattttcagtagagacaaggttgcgctatgttgcccaggctggtatggaactcctgtgcttaagcaatcctcatgcctcagcttcccaaagtgctgaggttacagctatgagccaccgcacccagcctacattccttcttatcaccgagaaacaggttgatcttcacaggtgtaatgagtatgaagggagtgccataagatattttttattttttatttatttattttttaatttaatttttttttttttgagatggagtcttgctctggcacccaggctagagtgcagtggtgcgatctcggctcactgcaacctctgcctcccaggttcaagcgattcttctgcctcagcttcccgaatagctgggattacaggtgcccaccaccacacccggctaatttttgtatttttagtagagacggagtttcaccatgttggccaggctggtctcaaactcctggcctcaggtgatccacccgcctcggcctcccaaagtgttgtgattacaagcatgagccatggtgccggcgggctgatttttttaatttttagtagagacaaagtctcactatattgcctaggttggtctcaaactgctgagctcaagcaatctgccggccatgttctctcaaagtgctgggattacaggcttgagcccctgcacccagcctgtaagatattttaaaatccacacttggccaagcgtggtggctcacacatataatcccagcactttgggaggccaaggtgggcggatcacaaggtcaggagttcgagaccagcctggccaatatggtgaaatcccatctctactaaaaatacaaaaactagccaggcgtgttggcttacacttatagtcccagctactcaggtggctgaggcaggagaatcacttgaaccaggaggcggagtttgcagtgagctgagatcacagcactgcacaccagcctcggcaacagagtgagactccgtctgaaaaaaaaaaaatccacgctgttttatatttctaacatggggtaggataccatcctccatgaaatagggtgcaagtgtgtgaaatgcagatgggaatcctgtcccagctagcctgacagagaagtgagcacttcctatggcaaattatgtaagaaaatattccccaggcagatgcttatgaaaagaatctgttcagcctgcaggtaagtgggagagcccttgctc

SEQ ID NO: 2 - DNA, 1230 bp, synthetic construct
  source 1..1230: mol_type = other DNA
atggtgggagctgacccaaccaggcccagaggacctctgtcttactgggctggcaggagaggacagggactggccgctatcttcctgctgctggtgtctgccgctgagagcgaggcccgggctgaggacgatttttccctggtgcagccactggtgacaatggagcagctgctgtgggtgagcggaaagcagatcggctccgtggacaccttccggatccctctgatcaccgctacaccacgcggcacactgctggccttcgctgaggctaggaagaagagcgccagcgacgagggcgctaagtttatcgctatgcggcgctccaccgatcagggatctacctggagctccacagctttcatcgtggacgatggagaggccagcgatggactgaacctgggcgctgtggtgaacgacgtggataccggcatcgtgtttctgatctacacactgtgcgcccacaaggtgaactgtcaggtggcttctaccatgctggtgtggagcaaggacgatggcatctcttggagcccccctaggaacctgtctgtggacatcggaacagagatgttcgcccctggcccaggaagcggcatccagaagcagagggagccaggaaagggccgcctgatcgtgtgcggacacggcaccctggagagagatggagtgttttgtctgctgtccgacgatcacggcgcctcttggcactacggaacaggcgtgtccggaatccctttcggccagccaaagcacgaccacgacttcaacccagacgagtgccagccatacgagctgcctgatggctccgtgatcatcaacgccaggaaccagaacaactaccactgccggtgtcgcatcgtgctgagatcttacgacgcttgtgataccctgaggcctagagacgtgacattcgatccagagctggtggacccagtggtggctgctggagctctggccacctctagcggcatcgtgttctttagcaacccagcccaccccgagttcagggtgaacctgaccctgagatggtccttttctaacggcacatcctggcagaaggagagggtgcaagtgtggccaggacctagcggctactcctctctgaccgccctggagaactccacagacggcaagaagcagccaccccagctgtttgtgctgtacgagaagggactgaaccgctacaccgagagcatctccatggtgaagatcagcgtgtacggaacactgtga

SEQ ID NO: 3 - DNA, 1104 bp, synthetic construct
  source 1..1104: mol_type = other DNA
gaggatgacttcagcctggtgcagccgctggtgaccatggagcagctgctgtgggtgagcgggaagcagatcggctctgtagacactttccgcatcccgctcatcacagccacccctcggggcacgctcctggccttcgctgaggccaggaaaaaatctgcatccgatgagggggccaagttcatcgccatgaggaggtccacggaccagggtagcacgtggtcctctacagccttcatcgtagacgatggggaggcctccgatggcctgaacctgggcgctgtggtgaacgatgtagacacagggatagtgttccttatctataccctctgtgctcacaaggtcaactgccaggtggcctctaccatgttggtttggagtaaggacgacggcatttcctggagcccaccccggaatctctctgtggatattggcacagagatgtttgcccctggacctggctcaggcattcagaaacagcgggagcctgggaagggccggctcattgtgtgtggacacgggacgctggagcgagatggggtcttctgtctcctcagtgatgaccacggtgcctcctggcactacggcactggagtgagcggcattccctttggccagcccaaacacgatcacgatttcaaccccgacgagtgccagccctacgagcttccagatggctcggtcatcatcaacgcccggaaccagaataactaccattgccgctgcaggatcgtcctccgcagctatgacgcctgtgacaccctcaggccccgggatgtgaccttcgaccctgagctcgtggaccctgtggtagctgcaggagcactagccaccagctccggcattgtcttcttctccaatccagcccaccctgagttccgagtgaacctgaccctgcgctggagtttcagcaatggtacatcctggcagaaggagagggtccaggtgtggccgggacccagcggctactcgtccctgacagccctggaaaacagcacggatggaaagaagcagcccccgcagctgttcgttctgtacgagaaaggcctgaaccggtacaccgagagcatctccatggtcaaaatcagcgtctacggcacgctc

SEQ ID NO: 4 - DNA, 1104 bp, Homo sapiens
  source 1..1104: mol_type = genomic DNA
gaaaacgattttggcctggtgcagccgctggtgaccatggaacagctgctgtgggtgagcggccgccagattggcagcgtggatacctttcgcattccgctgattaccgcgaccccgcgcggcaccctgctggcgtttgcggaagcgcgcaaaatgagcagcagcgatgaaggcgcgaaatttattgcgctgcgccgcagcatggatcagggcagcacctggagcccgaccgcgtttattgtgaacgatggcgatgtgccggatggcctgaacctgggcgcggtggtgagcgatgtggaaaccggcgtggtgtttctgttttatagcctgtgcgcgcataaagcgggctgccaggtggcgagcaccatgctggtgtggagcaaagatgatggcgtgagctggagcaccccgcgcaacctgagcctggatattggcaccgaagtgtttgcgccgggcccgggcagcggcattcagaaacagcgcgaaccgcgcaaaggccgcctgattgtgtgcggccatggcaccctggaacgcgatggcgtgttttgcctgctgagcgatgatcatggcgcgagctggcgctatggcagcggcgtgagcggcattccgtatggccagccgaaacaggaaaacgattttaacccggatgaatgccagccgtatgaactgccggatggcagcgtggtgattaacgcgcgcaaccagaacaactatcattgccattgccgcattgtgctgcgcagctatgatgcgtgcgataccctgcgcccgcgcgatgtgacctttgatccggaactggtggatccggtggtggcggcgggcgcggtggtgaccagcagcggcattgtgttttttagcaacccggcgcatccggaatttcgcgtgaacctgaccctgcgctggagctttagcaacggcaccagctggcgcaaagaaaccgtgcagctgtggccgggcccgagcggctatagcagcctggcgaccctggaaggcagcatggatggcgaagaacaggcgccgcagctgtatgtgctgtatgaaaaaggccgcaaccattataccgaaagcattagcgtggcgaaaattagcgtgtatggcaccctg

SEQ ID NO: 5 - protein, 415 aa, Homo sapiens
MTGERPSTALPDRRWGPRILGFWGGCRVWVFAAIFLLLSLAASWSKAENDFGLVQPLVTMEQLLWVSGRQIGSVDTFRIPLITATPRGTLLAFAEARKMSSSDEGAKFIALRRSMDQGSTWSPTAFIVNDGDVPDGLNLGAVVSDVETGVVFLFYSLCAHKAGCQVASTMLVWSKDDGVSWSTPRNLSLDIGTEVFAPGPGSGIQKQREPRKGRLIVCGHGTLERDGVFCLLSDDHGASWRYGSGVSGIPYGQPKQENDFNPDECQPYELPDGSVVINARNQNNYHCHCRIVLRSYDACDTLRPRDVTFDPELVDPVVAAGAVVTSSGIVFFSNPAHPEFRVNLTLRWSFSNGTSWRKETVQLWPGPSGYSSLATLEGSMDGEEQAPQLYVLYEKGRNHYTESISVAKISVYGTL

SEQ ID NO: 6 - protein, 356 aa, synthetic construct
MEQLLWVSGKQIGSVDTFRIPLITATPRGTLLAFAEARKKSASDEGAKFIAMRRSTDQGSTWSSTAFIVDDGEASDGLNLGAVVNDVDTGIVFLIYTLCAHKVNCQVASTMLVWSKDDGISWSPPRNLSVDIGTEMFAPGPGSGIQKQREPGKGRLIVCGHGTLERDGVFCLLSDDHGASWHYGTGVSGIPFGQPKHDHDFNPDECQPYELPDGSVIINARNQNNYHCRCRIVLRSYDACDTLRPRDVTFDPELVDPVVAAGALATSSGIVFFSNPAHPEFRVNLTLRWSFSNGTSWQKERVQVWPGPSGYSSLTALENSTDGKKQPPQLFVLYEKGLNRYTESISMVKISVYGTL

SEQ ID NO: 7 - protein, 409 aa, synthetic construct [sequence omitted]

SEQ ID NO: 8 - protein, 368 aa, Homo sapiens
ENDFGLVQPLVTMEQLLWVSGRQIGSVDTFRIPLITATPRGTLLAFAEARKMSSSDEGAKFIALRRSMDQGSTWSPTAFIVNDGDVPDGLNLGAVVSDVETGVVFLFYSLCAHKAGCQVASTMLVWSKDDGVSWSTPRNLSLDIGTEVFAPGPGSGIQKQREPRKGRLIVCGHGTLERDGVFCLLSDDHGASWRYGSGVSGIPYGQPKQENDFNPDECQPYELPDGSVVINARNQNNYHCHCRIVLRSYDACDTLRPRDVTFDPELVDPVVAAGAVVTSSGIVFFSNPAHPEFRVNLTLRWSFSNGTSWRKETVQLWPGPSGYSSLATLEGSMDGEEQAPQLYVLYEKGRNHYTESISVAKISVYGTL

SEQ ID NO: 9 - DNA, 1858 bp, Homo sapiens
  source 1..1858: mol_type = genomic DNA
agtccccgggcgcctcctggagagcaaggacgcgggggagcagagatgatccgagccgcgccgccgccgctgttcctgctgctgctgctgctgctgctgctagtgtcctgggcgtcccgaggcgaggcagcccccgaccaggacgagatccagcgcctccccgggctggccaagcagccgtctttccgccagtactccggctacctcaaaggctccggctccaagcacctccactactggtttgtggagtcccagaaggatcccgagaacagccctgtggtgctttggctcaatgggggtcccggctgcagctcactagatgggctcctcacagagcatggccccttcctggtccagccagatggtgtcaccctggagtacaacccctattcttggaatctgattgccaatgtgttatacctggagtccccagctggggtgggcttctcctactccgatgacaagttttatgcaactaatgacactgaggtcgcccagagcaattttgaggcccttcaagatttcttccgcctctttccggagtacaagaacaacaaacttttcctgaccggggagagctatgctggcatctacatccccaccctggccgtgctggtcatgcaggatcccagcatgaaccttcaggggctggctgtgggcaatggactctcctcctatgagcagaatgacaactccctggtctactttgcctactaccatggccttctggggaacaggctttggtcttctctccagacccactgctgctctcaaaacaagtgtaacttctatgacaacaaagacctggaatgcgtgaccaatcttcaggaagtggcccgcatcgtgggcaactctggcctcaacatctacaatctctatgccccgtgtgctggaggggtgcccagccattttaggtatgagaaggacactgttgtggtccaggatttgggcaacatcttcactcgcctgccactcaagcggatgtggcatcaggcactgctgcgctcaggggataaagtgcgcatggaccccccctgcaccaacacaacagctgcttccacctacctcaacaacccgtacgtgcggaaggccctcaacatcccggagcagctgccacaatgggacatgtgcaactttctggtaaacttacagtaccgccgtctctaccgaagcatgaactcccagtatctgaagctgcttagctcacagaaataccagatcctattatataatggagatgtagacatggcctgcaatttcatgggggatgagtggtttgtggattccctcaaccagaagatggaggtgcagcgccggccctggttagtgaagtacggggacagcggggagcagattgccggcttcgtgaaggagttctcccacatcgcctttctcacgatcaagggcgccggccacatggttcccaccgacaagcccctcgctgccttcaccatgttctcccgcttcctgaacaagcagccatactgatgaccacagcaaccagctccacggcctgatgcagcccctcccagcctctcccgctaggagagtcctcttctaagcaaagtgcccctgcaggccgggttctgccgccaggactgcccccttcccagagccctgtacatcccagactgggcccagggtctcccatagacagcctgggggcaagttagcactttattcccgcagcagttcctgaatggggtggcctggccccttctctgcttaaagaatgccctttatgatgcactgattccatcccaggaacccaacagagctcaggacagcccacagggaggtggtggacggactgtaattgatagattgattatggaattaaattgggtacagcttcaaa

SEQ ID NO: 10 - DNA, 1353 bp, synthetic construct
  source 1..1353: mol_type = other DNA
gctccggaccaggatgaaatcgattgtctccccggcctggccaagcagccctctttccggcaatactccggctacctcagagcatcggactccaagcacttccactactggtttgtggagtcgcagaacgacccaaagaacagccccgtggtgctttggcttaacgggggtcccggctgcagctcgctcgatgggctgcttacagagcacggcccctttctgatccagccagatggtgtcaccctggagtacaacccctatgcttggaacctgattgccaacgtgctgtatatcgagtccccagctggggtgggcttctcctactcggatgacaagatgtacgtgaccaatgacacagaggtggcggagaacaattatgaagcccttaaagacttcttccgcctctttccggaatacaaggacaacaaacttttcctgacaggagagagctatgctggcatctacatccccaccttggctgtactggtcatgcaggatcctagcatgaatcttcaggggctggctgtgggcaatggacttgcctcctatgagcagaacgacaactccctggtctactttgcctactaccatggccttctggggaacagactttggacttcactgcagacccactgctgcgctcagaacaagtgtaacttctatgacaacaaagacccagagtgtgtaaacaatctcctggaagtgtctcgaattgtgggcaaatctggcctcaacatctacaatctctatgctccgtgtgctggtggggtgcccggcagacatagatatgaggacacacttgtagtccaggattttggcaacatcttcactcgcctgccacttaagcggagatttcctgaggcactgatgcgttctggggacaaggtacgcttggatcctccctgcaccaacaccacagccccttccaactacctcaacaacccctatgttcggaaggctctccacatccccgagtcgctgccccgctgggacatgtgcaacttcttggtgaatttacagtaccgccgcctctaccaaagcatgaactcccagtacctgaagctgctcagttcacagaaataccagatcctgctctacaacggagatgtggacatggcctgcaacttcatgggcgatgagtggtttgtggattcgctcaaccagaagatggaggtgcagcgccggccctggctagtggactacggggagagcggagaacaggtagctggtttcgtgaaggagtgttcacacatcaccttcctcaccatcaagggtgccggacacatggtccccacggacaagcctcgagctgcttttaccatgttctcgaggttcctgaacaaagagccttac

SEQ ID NO: 11 - protein, 480 aa, Homo sapiens
MIRAAPPPLFLLLLLLLLLVSWASRGEAAPDQDEIQRLPGLAKQPSFRQYSGYLKGSGSKHLHYWFVESQKDPENSPVVLWLNGGPGCSSLDGLLTEHGPFLVQPDGVTLEYNPYSWNLIANVLYLESPAGVGFSYSDDKFYATNDTEVAQSNFEALQDFFRLFPEYKNNKLFLTGESYAGIYIPTLAVLVMQDPSMNLQGLAVGNGLSSYEQNDNSLVYFAYYHGLLGNRLWSSLQTHCCSQNKCNFYDNKDLECVTNLQEVARIVGNSGLNIYNLYAPCAGGVPSHFRYEKDTVVVQDLGNIFTRLPLKRMWHQALLRSGDKVRMDPPCTNTTAASTYLNNPYVRKALNIPEQLPQWDMCNFLVNLQYRRLYRSMNSQYLKLLSSQKYQILLYNGDVDMACNFMGDEWFVDSLNQKMEVQRRPWLVKYGDSGEQIAGFVKEFSHIAFLTIKGAGHMVPTDKPLAAFTMFSRFLNKQPY

SEQ ID NO: 12 - protein, 451 aa, synthetic construct
APDQDEIDCLPGLAKQPSFRQYSGYLRASDSKHFHYWFVESQNDPKNSPVVLWLNGGPGCSSLDGLLTEHGPFLIQPDGVTLEYNPYAWNLIANVLYIESPAGVGFSYSDDKMYVTNDTEVAENNYEALKDFFRLFPEYKDNKLFLTGESYAGIYIPTLAVLVMQDPSMNLQGLAVGNGLASYEQNDNSLVYFAYYHGLLGNRLWTSLQTHCCAQNKCNFYDNKDPECVNNLLEVSRIVGKSGLNIYNLYAPCAGGVPGRHRYEDTLVVQDFGNIFTRLPLKRRFPEALMRSGDKVRLDPPCTNTTAPSNYLNNPYVRKALHIPESLPRWDMCNFLVNLQYRRLYQSMNSQYLKLLSSQKYQILLYNGDVDMACNFMGDEWFVDSLNQKMEVQRRPWLVDYGESGEQVAGFVKECSHITFLTIKGAGHMVPTDKPRAAFTMFSRFLNKEPY

SEQ ID NO: 13 - DNA, 141 bp, synthetic construct
  source 1..141: mol_type = other DNA
atgaccggcgaacgcccgagcaccgcgctgccggatcgccgctggggcccgcgcattctgggcttttggggcggctgccgcgtgtgggtgtttgcggcgatttttctgctgctgagcctggcggcgagctggagcaaagcg

SEQ ID NO: 14 - DNA, 123 bp, synthetic construct
  source 1..123: mol_type = other DNA
atgacttccagtccaaaggcgcctcctggagagcaaggacgcaaggaagcagagatgcccggaaccgcgctgtctccactgctcttgttgctgctcctgtcctgggcgtcccggaacgaagca

SEQ ID NO: 15 - DNA, 75 bp, synthetic construct
  source 1..75: mol_type = other DNA
atgcgacccccgcgtccctcctcagctatgctgacgttttttgctgcgttcttggccgcgcccttggcgctggct

SEQ ID NO: 16 - protein, 47 aa, synthetic construct
MTGERPSTALPDRRWGPRILGFWGGCRVWVFAAIFLLLSLAASWSKA

SEQ ID NO: 17 - protein, 41 aa, synthetic construct
MTSSPKAPPGEQGRKEAEMPGTALSPLLLLLLLSWASRNEA

SEQ ID NO: 18 - protein, 25 aa, synthetic construct
MRPPRPSSAMLTFFAAFLAAPLALA

SEQ ID NO: 19 - DNA, 1182 bp, synthetic construct
  source 1..1182: mol_type = other DNA
atgcggccccctaggcccagctccgccatgctgaccttctttgctgctttcctggctgctccactggccctggctgaggacgatttttctctggtgcagcccctggtgacaatggagcagctgctgtgggtgagcggaaagcagatcggctccgtggacaccttcaggatccccctgatcaccgccacacctagaggcacactgctggccttcgctgaggccaggaagaagagcgccagcgacgagggcgctaagtttatcgccatgaggagatccaccgatcagggctctacctggtctagcacagctttcatcgtggacgatggagaggcctccgatggactgaacctgggcgctgtggtgaacgacgtggataccggcatcgtgtttctgatctacacactgtgcgcccacaaggtgaactgtcaggtggcttctaccatgctggtgtggagcaaggacgatggcatctcctggtctccaccccggaacctgtctgtggacatcggaacagagatgttcgctccaggacctggaagcggaatccagaagcagagggagcctggaaagggcagactgatcgtgtgcggacacggcaccctggagagggatggagtgttttgtctgctgtccgacgatcacggcgcctcttggcactacggaacaggcgtgagcggaatccccttcggccagcctaagcacgaccacgacttcaaccctgacgagtgccagccatacgagctgcccgatggctccgtgatcatcaacgcccggaaccagaacaactaccactgccggtgtcgcatcgtgctgcgctcttacgacgcttgtgataccctgaggccaagagacgtgacattcgatcctgagctggtggacccagtggtggctgctggagctctggccacctcctctggcatcgtgttctttagcaaccctgcccacccagagttccgggtgaacctgaccctgcgctggagcttttccaacggcacatcctggcagaaggagagggtgcaagtgtggccaggaccaagcggctacagctccctgaccgctctggagaactccacagacggcaagaagcagcctccacagctgtttgtgctgtacgagaagggactgaacagatacaccgagtctatcagcatggtgaagatctccgtgtacggaacactgtga

SEQ ID NO: 20 - protein, 393 aa, synthetic construct
MRPPRPSSAMLTFFAAFLAAPLALAEDDFSLVQPLVTMEQLLWVSGKQIGSVDTFRIPLITATPRGTLLAFAEARKKSASDEGAKFIAMRRSTDQGSTWSSTAFIVDDGEASDGLNLGAVVNDVDTGIVFLIYTLCAHKVNCQVASTMLVWSKDDGISWSPPRNLSVDIGTEMFAPGPGSGIQKQREPGKGRLIVCGHGTLERDGVFCLLSDDHGASWHYGTGVSGIPFGQPKHDHDFNPDECQPYELPDGSVIINARNQNNYHCRCRIVLRSYDACDTLRPRDVTFDPELVDPVVAAGALATSSGIVFFSNPAHPEFRVNLTLRWSFSNGTSWQKERVQVWPGPSGYSSLTALENSTDGKKQPPQLFVLYEKGLNRYTESISMVKISVYGTL

SEQ ID NO: 21 - DNA, 6220 bp, synthetic construct
  source 1..6220: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgatatcaagcttgctagccgccaccatgcggccccctaggcccagctccgccatgctgaccttctttgctgctttcctggctgctccactggccctggctgaggacgatttttctctggtgcagcccctggtgacaatggagcagctgctgtgggtgagcggaaagcagatcggctccgtggacaccttcaggatccccctgatcaccgccacacctagaggcacactgctggccttcgctgaggccaggaagaagagcgccagcgacgagggcgctaagtttatcgccatgaggagatccaccgatcagggctctacctggtctagcacagctttcatcgtggacgatggagaggcctccgatggactgaacctgggcgctgtggtgaacgacgtggataccggcatcgtgtttctgatctacacactgtgcgcccacaaggtgaactgtcaggtggcttctaccatgctggtgtggagcaaggacgatggcatctcctggtctccaccccggaacctgtctgtggacatcggaacagagatgttcgctccaggacctggaagcggaatccagaagcagagggagcctggaaagggcagactgatcgtgtgcggacacggcaccctggagagggatggagtgttttgtctgctgtccgacgatcacggcgcctcttggcactacggaacaggcgtgagcggaatccccttcggccagcctaagcacgaccacgacttcaaccctgacgagtgccagccatacgagctgcccgatggctccgtgatcatcaacgcccggaaccagaacaactaccactgccggtgtcgcatcgtgctgcgctcttacgacgcttgtgataccctgaggccaagagacgtgacattcgatcctgagctggtggacccagtggtggctgctggagctctggccacctcctctggcatcgtgttctttagcaaccctgcccacccagagttccgggtgaacctgaccctgcgctggagcttttccaacggcacatcctggcagaaggagagggtgcaagtgtggccaggaccaagcggctacagctccctgaccgctctggagaactccacagacggcaagaagcagcctccacagctgtttgtgctgtacgagaagggactgaacagatacaccgagtctatcagcatggtgaagatctccgtgtacggaacactgtgactcgagtttttttttgcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcc

SEQ ID NO: 22 - DNA, 6268 bp, synthetic construct
  source 1..6268: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccagatcttcaatattggccattagccatattattcattggttatatagcataaatcaatattggctattggccattgcatacgttgtatctatatcataatatgtacatttatattggctcatgtccaatatgaccgccatgttggcattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattcgatatcaagcttgctagccgccaccatggtgggagctgacccaaccaggcccagaggacctctgtcttactgggctggcaggagaggacagggactggccgctatcttcctgctgctggtgtctgccgctgagagcgaggcccgggctgaggacgatttttccctggtgcagccactggtgacaatggagcagctgctgtgggtgagcggaaagcagatcggctccgtggacaccttccggatccctctgatcaccgctacaccacgcggcacactgctggccttcgctgaggctaggaagaagagcgccagcgacgagggcgctaagtttatcgctatgcggcgctccaccgatcagggatctacctggagctccacagctttcatcgtggacgatggagaggccagcgatggactgaacctgggcgctgtggtgaacgacgtggataccggcatcgtgtttctgatctacacactgtgcgcccacaaggtgaactgtcaggtggcttctaccatgctggtgtggagcaaggacgatggcatctcttggagcccccctaggaacctgtctgtggacatcggaacagagatgttcgcccctggcccaggaagcggcatccagaagcagagggagccaggaaagggccgcctgatcgtgtgcggacacggcaccctggagagagatggagtgttttgtctgctgtccgacgatcacggcgcctcttggcactacggaacaggcgtgtccggaatccctttcggccagccaaagcacgaccacgacttcaacccagacgagtgccagccatacgagctgcctgatggctccgtgatcatcaacgccaggaaccagaacaactaccactgccggtgtcgcatcgtgctgagatcttacgacgcttgtgataccctgaggcctagagacgtgacattcgatccagagctggtggacccagtggtggctgctggagctctggccacctctagcggcatcgtgttctttagcaacccagcccaccccgagttcagggtgaacctgaccctgagatggtccttttctaacggcacatcctggcagaaggagagggtgcaagtgtggccaggacctagcggctactcctctctgaccgccctggagaactccacagacggcaagaagcagccaccccagctgtttgtgctgtacgagaagggactgaaccgctacaccgagagcatctccatggtgaagatcagcgtgtacggaacactgtgactcgagtttttttttgcggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggagatgtgggaggttttttaaagcaagtaaaacctctacaaatgtggtaaaatcgataggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaataggccgaaatcggcaaaatcccttataaatcaaaagaatagcccgagatagggttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcc

SEQ ID NO: 23 - DNA, 7424 bp, synthetic construct
  source 1..7424: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattctaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcgagtcagtaaggctctttgttcaggaacctcgagaacatggtaaaagcagctcgaggcttgtccgtggggaccatgtgtccggcacccttgatggtgaggaaggtgatgtgtgaacactccttcacgaaaccagctacctgttctccgctctccccgtagtccactagccagggccggcgctgcacctccatcttctggttgagcgaatccacaaaccactcatcgcccatgaagttgcaggccatgtccacatctccgttgtagagcaggatctggtatttctgtgaactgagcagcttcaggtactgggagttcatgctttggtagaggcggcggtactgtaaattcaccaagaagttgcacatgtcccagcggggcagcgactcggggatgtggagagccttccgaacataggggttgttgaggtagttggaaggggctgtggtgttggtgcagggaggatccaagcgtaccttgtccccagaacgcatcagtgcctcaggaaatctccgcttaagtggcaggcgagtgaagatgttgccaaaatcctggactacaagtgtgtcctcatatctatgtctgccgggcaccccaccagcacacggagcatagagattgtagatgttgaggccagatttgcccacaattcgagacacttccaggagattgtttacacactctgggtctttgttgtcatagaagttacacttgttctgagcgcagcagtgggtctgcagtgaagtccaaagtctgttccccagaaggccatggtagtaggcaaagtagaccagggagttgtcgttctgctcataggaggcaagtccattgcccacagccagcccctgaagattcatgctaggatcctgcatgaccagtacagccaaggtggggatgtagatgccagcatagctctctcctgtcaggaaaagtttgttgtccttgtattccggaaagaggcggaagaagtctttaagggcttcataattgttctccgccacctctgtgtcattggtcacgtacatcttgtcatccgagtaggagaagcccaccccagctggggactcgatatacagcacgttggcaatcaggttccaagcataggggttgtactccagggtgacaccatctggctggatcagaaaggggccgtgctctgtaagcagcccatcgagcgagctgcagccgggacccccgttaagccaaagcaccacggggctgttctttgggtcgttctgcgactccacaaaccagtagtggaagtgcttggagtccgatgctctgaggtagccggagtattgccggaaagagggctgcttggccaggccggggagacaatcgatttcatcctggtccggagctgcttcgttccgggacgcccaggacaggagcagcaacaagagcagtggagacagcgcggttccgggcatctctgcttccttgcgtccttgctctccaggaggcgcctttggactggaagtcatggtggcggctagcgagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaaactagcgctagagcttgctcccgcccgccgcgcgcttcgctttttatagggccgccgccgccgccgcctcgccataaaaggaaactttcggagcgcgccgctctgattggctgccgccgcacctctccgcctcgccccgccccgcccctcgcccccatcgctgcacaaaataattaaaaaataaataaatacaaaattgggggtggggaggggggggagatggggagagtgaagcagaacgtggcctcggatcccccgggctgcagtattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctcgctagcactagtggcgcgcccaggtaagtttagtctttttgtcttttatttcaggtcccggatccggtggtggtgcaaatcaaagaactgctcctcagtggatgttgcctttacttctaggcctgtacggaagtggctagccgccaccatgcgacccccgcgtccctcctcagctatgctgacgttttttgctgcgttcttggccgcgcccttggcgctggctgaggatgacttcagcctggtgcagccgctggtgaccatggagcagctgctgtgggtgagcgggaagcagatcggctctgtagacactttccgcatcccgctcatcacagccacccctcggggcacgctcctggccttcgctgaggccaggaaaaaatctgcatccgatgagggggccaagttcatcgccatgaggaggtccacggaccagggtagcacgtggtcctctacagccttcatcgtagacgatggggaggcctccgatggcctgaacctgggcgctgtggtgaacgatgtagacacagggatagtgttccttatctataccctctgtgctcacaaggtcaactgccaggtggcctctaccatgttggtttggagtaaggacgacggcatttcctggagcccaccccggaatctctctgtggatattggcacagagatgtttgcccctggacctggctcaggcattcagaaacagcgggagcctgggaagggccggctcattgtgtgtggacacgggacgctggagcgagatggggtcttctgtctcctcagtgatgaccacggtgcctcctggcactacggcactggagtgagcggcattccctttggccagcccaaacacgatcacgatttcaaccccgacgagtgccagccctacgagcttccagatggctcggtcatcatcaacgcccggaaccagaataactaccattgccgctgcaggatcgtcctccgcagctatgacgcctgtgacaccctcaggccccgggatgtgaccttcgaccctgagctcgtggaccctgtggtagctgcaggagcactagccaccagctccggcattgtcttcttctccaatccagcccaccctgagttccgagtgaacctgaccctgcgctggagtttcagcaatggtacatcctggcagaaggagagggtccaggtgtggccgggacccagcggctactcgtccctgacagccctggaaaacagcacggatggaaagaagcagcccccgcagctgttcgttctgtacgagaaaggcctgaaccggtacaccgagagcatctccatggtcaaaatcagcgtctacggcacgctctgactcgagcggccgctctagagatctttttccctctgccaaaaattatggggacatcatgaagccccttgagcatctgacttctggctaataaaggaaatttattttcattgcaatagtgtgttggaattttttgtgtctctcactcggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccatgcagccagctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgggctgcaggggggggggggggggggtgggggggggggggggggg

SEQ ID NO: 24 - DNA, 7473 bp, synthetic construct
  source 1..7473: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattctaccacatttgtagaggttttacttgctttaaaaaacctcccacatctccccctgaacctgaaacataaaatgaatgcaattgttgttgttaacttgtttattgcagcttataatggttacaaataaagcaatagcatcacaaatttcacaaataaagcatttttttcactgcattctagttgtggtttgtccaaactcatcaatgtatcttatcatgtctgtcgagtcagtaaggctctttgttcaggaacctcgagaacatggtaaaagcagctcgaggcttgtccgtggggaccatgtgtccggcacccttgatggtgaggaaggtgatgtgtgaacactccttcacgaaaccagctacctgttctccgctctccccgtagtccactagccagggccggcgctgcacctccatcttctggttgagcgaatccacaaaccactcatcgcccatgaagttgcaggccatgtccacatctccgttgtagagcaggatctggtatttctgtgaactgagcagcttcaggtactgggagttcatgctttggtagaggcggcggtactgtaaattcaccaagaagttgcacatgtcccagcggggcagcgactcggggatgtggagagccttccgaacataggggttgttgaggtagttggaaggggctgtggtgttggtgcagggaggatccaagcgtaccttgtccccagaacgcatcagtgcctcaggaaatctccgcttaagtggcaggcgagtgaagatgttgccaaaatcctggactacaagtgtgtcctcatatctatgtctgccgggcaccccaccagcacacggagcatagagattgtagatgttgaggccagatttgcccacaattcgagacacttccaggagattgtttacacactctgggtctttgttgtcatagaagttacacttgttctgagcgcagcagtgggtctgcagtgaagtccaaagtctgttccccagaaggccatggtagtaggcaaagtagaccagggagttgtcgttctgctcataggaggcaagtccattgcccacagccagcccctgaagattcatgctaggatcctgcatgaccagtacagccaaggtggggatgtagatgccagcatagctctctcctgtcaggaaaagtttgttgtccttgtattccggaaagaggcggaagaagtctttaagggcttcataattgttctccgccacctctgtgtcattggtcacgtacatcttgtcatccgagtaggagaagcccaccccagctggggactcgatatacagcacgttggcaatcaggttccaagcataggggttgtactccagggtgacaccatctggctggatcagaaaggggccgtgctctgtaagcagcccatcgagcgagctgcagccgggacccccgttaagccaaagcaccacggggctgttctttgggtcgttctgcgactccacaaaccagtagtggaagtgcttggagtccgatgctctgaggtagccggagtattgccggaaagagggctgcttggccaggccggggagacaatcgatttcatcctggtccggagctgcttcgttccgggacgcccaggacaggagcagcaacaagagcagtggagacagcgcggttccgggcatctctgcttccttgcgtccttgctctccaggaggcgcctttggactggaagtcatggtggcggctagcgagtggacacctgtggagagaaaggcaaagtggatgtcagtaagaccaataggtgcctatcagaaacgcaagagtcttctctgtctcgacaagcccagtttctattggtctccttaaacctgtcttgtaaccttgatacttacctgcccagtgcctcacgaccaaactagcgctagagcttgctcccgcccgccgcgcgcttcgctttttatagggccgccgccgccgccgcctcgccataaaaggaaactttcggagcgcgccgctctgattggctgccgccgcacctctccgcctcgccccgccccgcccctcgcccccatcgctgcacaaaataattaaaaaataaataaatacaaaattgggggtggggaggggggggagatggggagagtgaagcagaacgtggcctcggatcccccgggctgcagtattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgtcgaggccacgttctgcttcactctccccatctcccccccctccccacccccaattttgtatttatttattttttaattattttgtgcagcgatgggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagcaagctcgctagcactagtggcgcgcccaggtaagtttagtctttttgtcttttatttcaggtcccggatccggtggtggtgcaaatcaaagaactgctcctcagtggatgttgcctttacttctaggcctgtacggaagtggctagccgccaccatggtgggggcagacccgaccagaccccggggaccgctgagctattgggcgggccgtcggggtcaggggctcgcagcgatcttcctgctcctggtgtccgcggcggaatccgaggccagggcagaggatgacttcagcctggtgcagccgctggtgaccatggagcagctgctgtgggtgagcgggaagcagatcggctctgtagacactttccgcatcccgctcatcacagccacccctcggggcacgctcctggccttcgctgaggccaggaaaaaatctgcatccgatgagggggccaagttcatcgccatgaggaggtccacggaccagggtagcacgtggtcctctacagccttcatcgtagacgatggggaggcctccgatggcctgaacctgggcgctgtggtgaacgatgtagacacagggatagtgttccttatctataccctctgtgctcacaaggtcaactgccaggtggcctctaccatgttggtttggagtaaggacgacggcatttcctggagcccaccccggaatctctctgtggatattggcacagagatgtttgcccctggacctggctcaggcattcagaaacagcgggagcctgggaagggccggctcattgtgtgtggacacgggacgctggagcgagatggggtcttctgtctcctcagtgatgaccacggtgcctcctggcactacggcactggagtgagcggcattccctttggccagcccaaacacgatcacgatttcaaccccgacgagtgccagccctacgagcttccagatggctcggtcatcatcaacgcccggaaccagaataactaccattgccgctgcaggatcgtcctccgcagctatgacgcctgtgacaccctcaggccccgggatgtgaccttcgaccctgagctcgtggaccctgtggtagctgcaggagcactagccaccagctccggcattgtcttcttctccaatccagcccaccctgagttccgagtgaacctgaccctgcgctggagtttcagcaatggtacatcctggcagaaggagagggtccaggtgtggccgggacccagcggctactcgtccctgacagccctggaaaacagcacggatggaaagaagcagcccccgcagctgttcgttctgtacgagaaaggcctgaaccggtacaccgagagcatctccatggtcaaaatcagcgtctacggcacgctctgactcgaggcggccgctctagagatctttttccctctgccaaaaattatggggacatcatgaagccccttgagcatctgacttctggctaataaaggaaatttattttcattgcaatagtgtgttggaattttttgtgtctctcactcggcatgctggggagagatctaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccatgcagccagctggcgtaatagcgaagaggcccgcaccgatcgcccttcccaacagttgcgtagcctgaatggcgaatggcgcgacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgattagggtgatggttcacgtagtgggccatcgccctgatagacggtttttcgccctttgacgttggagtccacgttctttaatagtggactcttgttccaaactggaacaacactcaaccctatctcggtctattcttttgatttataagggattttgccgatttcggcctattggttaaaaaatgagctgatttaacaaaaatttaacgcgaattttaacaaaatattaacgtttacaatttcctgatgcggtattttctccttacgcatctgtgcggtatttcacaccgcatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgggctgcaggggggggggggggggggtgggggggggggggggggg

(19) United States
(12) Patent Application Publication
(10) Pub. No.: US 2025/0135033 A1
(43) Pub. Date: May 1, 2025

(54) COMPOSITIONS AND METHODS FOR TREATING SIALIDOSIS

(71) Applicant: University of Massachusetts, Boston, MA (US)

(72) Inventors: Heather Gray-Edwards, Worcester, MA (US); Miguel Sena Esteves, Worcester, MA (US); Jillian Gallagher, Worcester, MA (US)

(73) Assignee: University of Massachusetts, Boston, MA (US)

(21) Appl. No.: 18/834,366

(22) PCT Filed: Jan. 31, 2023
(86) PCT No.: PCT/US2023/061644

§ 371 Date: Jul. 30, 2024

Related U.S. Application Data
(60) Provisional application No. 63/304,802, filed on Jan. 31, 2022.

(51) Int. Cl. A61K 48/00; A61K 38/47; A61K 38/48; A61P 3/00; C12N 15/86

(52) U.S. Cl. CPC A61K 48/005; A61K 38/47; A61K 38/4813; A61P 3/00; C12N 15/86; C12Y 302/01018; C12Y 302/01023; C12Y 304/16005; C12N 2750/14122; C12N 2750/14143; C12N 2830/15

(57) ABSTRACT

In some aspects the disclosure provides compositions and methods for promoting expression of functional NEU1 protein in a subject. In other aspects, the disclosure provides compositions and methods for treating sialidosis, galactosialidosis, and/or Alzheimer's Disease in a subject having or suspected of having sialidosis, galactosialidosis, and/or Alzheimer's Disease.

### RELATED APPLICATION

This application is a national stage filing under 35 U.S.C. § 371 of international PCT application PCT/US2023/061644, filed Jan. 31, 2023, which claims priority under 35 U.S.C. § 119(e) to U.S. provisional patent application, U.S. Ser. No. 63/304,802, filed Jan. 31, 2022, the entire contents of each of which are incorporated by reference herein.

### REFERENCE TO AN ELECTRONIC SEQUENCE LISTING

The contents of the electronic sequence listing (U012070168US01-SEQ-KZM.xml; Size: 63,566 bytes; and Date of Creation: Jul. 29, 2024) is herein incorporated by reference in its entirety.

### BACKGROUND OF INVENTION

Sialidosis is a rare neurodegenerative disease caused by deficiency of the sialic acid-cleaving enzyme Neuraminidase 1 (NEU1) and progressive buildup of sialylated glycopeptides and oligosaccharides in tissues and body fluids. Type 1 sialidosis is the attenuated non-neuropathic form of the disease, also known as cherry red spot myoclonus syndrome. Clinical signs manifest in the second decade of life with gait abnormalities, decreased visual acuity due to bilateral macular cherry red spots, involuntary myoclonus, retina cherry red spot, ataxia, loss of visual acuity, nystagmus, seizures and hyperreflexia. It is fatal in the 3rd to 5th decade of life. Type 1 patients have abnormal ophthalmic exam, cortical and cerebellar atrophy on MRI, epileptic events on electroencephalography (EEG) and myoclonus on electromyography (EMG). Type 2 sialidosis is a more aggressive form of the disease and is further subdivided into infantile/juvenile or congenital/hydropic forms based on the age of onset. Children with the infantile/juvenile form also develop myoclonus and seizures like the type 1 patients described above, but also develop developmental delay, milestone loss, intellectual disability and hearing and vision loss. Type 2 patients also suffer from systemic disease similar to the mucopolysaccharidoses including hepatosplenomegaly, dysostosis multiplex with joint disease, corneal clouding, facial edema and short stature. Infantile and juvenile forms of this disease are fatal in the first and second decades of life, respectively. These patients also exhibit abnormalities on EEG and MRI, as described above, and exhibit oligosacchariduria. Patients with the congenital form are stillborn or die shortly after birth with hydrops fetalis and ascites being common. There is no effective therapy for sialidosis.

### SUMMARY OF INVENTION

According to some aspects, the disclosure provides compositions and methods for delivering a transgene to a subject. In some aspects, the disclosure provides a recombinant adeno-associated virus (rAAV) comprising a capsid and a nucleic acid comprising a promoter and a sequence encoding NEU1. In some embodiments, the sequence encoding NEU1 is a mammalian sequence. In some embodiments, the mammalian sequence is a human, mouse, rat, goat, or sheep sequence.

In some embodiments, the nucleic acid does not include a signal peptide (e.g., a native NEU1 signal peptide). In some embodiments, the nucleic acid further comprises a sequence encoding a signal peptide, optionally wherein the sequence encoding a signal peptide is operably linked to the sequence encoding NEU1. In some embodiments, the signal peptide is a native NEU1 signal peptide or a variant thereof, optionally wherein the native NEU1 signal peptide comprises the amino acid sequence set forth in SEQ ID NO: 16. In some embodiments, the signal peptide is a signal peptide derived from a lysosomal protein. In some embodiments, the signal peptide is an iduronidase (IDUA) signal peptide, optionally wherein the IDUA signal peptide comprises the amino acid sequence set forth in SEQ ID NO: 18.

In some embodiments, the promoter is a chicken beta-actin (CBA) promoter, an enhanced chicken beta-actin promoter, a retroviral Rous sarcoma virus (RSV) long terminal repeat (LTR) promoter, a cytomegalovirus (CMV) promoter, a Simian vacuolating virus 40 (SV40) promoter, a dihydrofolate reductase promoter, a beta-actin promoter, a phosphoglycerol kinase (PGK) promoter, a EF1alpha promoter, or a U6 promoter.

In some embodiments, the sequence encoding NEU1 comprises the nucleotide sequence set forth in any one of SEQ ID NOs: 1-4. In some embodiments, the sequence encoding NEU1 encodes a NEU1 protein comprising an amino acid sequence as set forth in any one of SEQ ID NOs: 5-8.

In some embodiments, the sequence encoding NEU1 is a codon-optimized human NEU1 sequence.

In some embodiments, the nucleic acid further comprises a sequence encoding cathepsin A. In some embodiments, the nucleic acid comprises a first expression cassette engineered to express NEU1 and a second expression cassette engineered to express cathepsin A.

In some embodiments, the sequence encoding cathepsin A comprises the nucleotide sequence set forth in any one of SEQ ID NOs: 9-10. In some embodiments, the sequence encoding cathepsin A encodes a cathepsin A protein comprising the amino acid sequence set forth in any one of SEQ ID NOs: 11-12.

In some embodiments, the nucleic acid further comprises one or more enhancer sequences, optionally a cytomegalovirus (CMV) enhancer sequence and/or an SV40 enhancer sequence.

In some embodiments, the nucleic acid comprises one or more ITRs, wherein each ITR is selected from the group consisting of AAV1 ITR, AAV2 ITR, AAV3 ITR, AAV4 ITR, AAV5 ITR, and AAV6 ITR.

In some embodiments, the capsid is an AAV1, AAV2, AAV3, AAV4, AAV5, AAV6, AAV7, AAV8, or AAV9 capsid protein.

In some aspects, the disclosure provides a pharmaceutical composition comprising (i) an rAAV as described herein; and (ii) a pharmaceutically acceptable excipient or solution.

In some aspects, the disclosure provides an isolated nucleic acid comprising the sequence as set forth in any one of SEQ ID NOs: 21-24.

In some aspects, the disclosure provides a host cell comprising an rAAV as described herein or an isolated nucleic acid construct as described herein. In some embodiments, the cell is a mammalian cell, optionally a human cell, bacterial cell, yeast cell, or insect cell.

In some aspects, the disclosure provides a method for promoting expression of neuraminidase 1 (NEU1) protein in a subject. In some embodiments, a method for promoting expression of neuraminidase 1 (NEU1) protein in a subject comprises administering to the subject an rAAV as described herein, a pharmaceutical composition as described herein, an isolated nucleic acid as described herein, or a host cell as described herein.

In some aspects, the disclosure provides a method for promoting expression of multiprotein complex in the lysosome of a target cell in a subject. In some embodiments, the multiprotein complex comprises Neuraminidase 1 (NEU1), acid beta-galactosidase (GLB1) and cathepsin A protein. In some embodiments, a method for promoting expression of multiprotein complex in the lysosome of a target cell in a subject comprises administering to the subject an rAAV as described herein, a pharmaceutical composition as described herein, an isolated nucleic acid as described herein, or a host cell as described herein.

In some aspects, the disclosure provides a method for treating sialidosis or galactosialidosis in a subject in need thereof. In some embodiments, a method for treating sialidosis or galactosialidosis comprises administering to the subject an rAAV as described herein, a pharmaceutical composition as described herein, an isolated nucleic acid as described herein, or a host cell as described herein.

In some embodiments, a subject has Type 1 sialidosis or Type 2 sialidosis. In some embodiments, the subject has one or more mutations in an endogenous NEU1 gene and/or endogenous CTSA gene. In some embodiments, the subject has a deletion in exon 2, exon 5, exon 6, and/or a A319V substitution.

In some aspects, the disclosure provides a method for treating Alzheimer's Disease in a subject in need thereof, the method comprising administering to the subject an rAAV as described herein, a pharmaceutical composition as described herein, an isolated nucleic acid as described herein, or a host cell as described herein.

In some embodiments, administration of an rAAV vector is by systemic injection.

These and other aspects and embodiments will be described in greater detail herein. The description of some exemplary embodiments of the disclosure are provided for illustration purposes only and not meant to be limiting. Additional compositions and methods are also embraced by this disclosure.

The summary above is meant to illustrate, in a non-limiting manner, some of the embodiments, advantages, features, and uses of the technology disclosed herein. Other embodiments, advantages, features, and uses of the technology disclosed herein will be apparent from the Detailed Description, Drawings, Examples, and Claims.

### BRIEF DESCRIPTION OF THE DRAWINGS

FIG. 1 shows a mapping of the exons and introns of human NEU1 gene. Shown at the top of the gene schematic are mutations known to be associated with type I sialidosis (e.g., A319V substitution). Additionally, it is known that deletions within exon 2 are associated with type I sialidosis (as indicated by the large ‘X’ over exon 2). Shown at the bottom of the gene schematic are mutations known to be associated with type II sialidosis.

FIGS. 2A-2G show schematics depicting exemplary embodiments of a recombinant adeno-associated virus (AAV) construct encoding Neuraminidase 1 (NEU1).

FIG. 3 shows representative data indicating the ability of rAAVs comprising a sequence encoding NEU1 to (i) modulate NEU1 protein activity in the liver and kidneys; and (ii) transduce the liver and kidneys, following systematic injection of a mouse model.

FIGS. 4A-4D show representative data indicating the ability of rAAVs comprising a sequence encoding NEU1 (AAV9-NEU1, AAV9-CTSA-BiCB6-NEU1, and AAV9-IDUA-NEU1) to (i) modulate NEU1 protein activity in the liver (FIG. 4A) and kidneys (FIG. 4B); and (ii) transduce the liver (FIG. 4C) and kidneys (FIG. 4D), following systematic injection of a mouse model.

FIG. 5A shows an animal study design in which neonatal mice were transduced with an rAAV encoding NEU1 (AAV9-NEU1, AAV9-CTSA-BiCB6-NEU1, and AAV9-IDUA-NEU1) and evaluated for survival, toxicity, behavior, NEU1 enzymatic activity, sialic acid levels, vector genome distribution, histology and immunohistochemical analyses over the course of 8 months until a humane endpoint is reached.

FIG. 5B shows the weight of mice one month after injection with an rAAV encoding NEU1.

FIGS. 6A-6B provide sequence maps indicating the target regions for the guide RNAs targeting exons 2, 5 and 6 of NEU1 for generation of a sheep model system.

### DETAILED DESCRIPTION OF INVENTION

In some aspects, the disclosure relates to compositions and methods for delivering transgenes encoding certain proteins such as neuraminidase (NEU1) and cathepsin A to a subject. In some embodiments, compositions are useful for methods of treating lysosomal diseases such as sialidosis, an autosomal recessive inherited disorder, that is caused by α-N-acetyl neuraminidase (NEU1) deficiency. In some embodiments, the disclosure relates to AAV-mediated gene replacement therapy for sialidosis. In some embodiments, the NEU1 deficiency results from one or more mutations in the neuraminidase gene (NEU1). In some aspects, the disclosure relates to compositions and methods for treating galactosialidosis, an autosomal recessive inherited disorder, that is caused by α-N-acetyl neuraminidase (NEU1) deficiency and a beta-galactosidase (GLB1) deficiency. In some embodiments, the disclosure relates to AAV-mediated gene replacement therapy for galactosialidosis. In some embodiments, the NEU1 deficiency results from one or more mutations in the neuraminidase gene (NEU1). In some embodiments, the GLB1 deficiency results from one or more mutations in the beta-galactosidase gene (GLB1). In some embodiments, the GLB1 deficiency results from one or more mutations in the cathepsin A gene (CTSA).

Mutations in the CTSA gene, which codes for cathepsin A (also known as protective protein or PPCA), can result in a double enzymatic deficiency in acid beta-galactosidase and neuraminidase activities even if both genes for these enzymes (GLB1 and NEU1, respectively) are normal. In some embodiments, AAV vectors are provided expressing a codon-optimized human NEU1 gene. In some embodiments, the AAV vectors further express a CTSA gene (e.g., a codon-optimized human CTSA). In some embodiments, the rAAV is delivered via systemic injection.

Neuraminidase protein (also known as Sialidase 1) exists in the lysosome as a megadalton multiprotein complex with acid beta-galactosidase (GLB1) and cathepsin A (protective protein or PPCA). Transport of neuraminidase from the endoplasmic reticulum where it is synthesized to the lysosome is dependent on its interaction with PPCA. Mutations in the CTSA gene, which codes for PPCA, results in a double enzymatic deficiency in acid beta-galactosidase and neuraminidase activities although both genes for these enzymes (GLB1 and NEU1, respectively) are normal, and hence why the CTSA protein is commonly known as protective protein/cathepsin A (PPCA). The disease caused by mutations in the CTSA gene is known as galactosialidosis. The NEU1 reliance on PPCA for intracellular transport indicates that overexpression of NEU1 alone may not be sufficient to achieve efficient secretion of the protein to enable cross-correction of from genetically engineered cells to all other cells in the central nervous system or elsewhere.

In some embodiments, the endogenous NEU1 gene in a subject having sialidosis has one or more mutations as described in Khan, A. and C. Sergi, “Sialidosis: A Review of Morphology and Molecular Biology of a Rare Pediatric Disorder” Diagnostics (Basel). 2018 June; 8(2): 29. In some embodiments, the endogenous NEU1 gene in a subject having sialidosis or galactosialidosis has one or more mutations as described in OMIM entry 256550, entitled “NEURAMINIDASE DEFICIENCY.” In some embodiments, the endogenous NEU1 gene in a subject having galactosialidosis has one or more mutations as described in OMIM entry 256540, entitled “GALACTOSIALIDOSIS.”

In some aspects, the disclosure relates to compositions and methods for treating Alzheimer's Disease, a progressive form of dementia, that is associated with decreased expression of NEU1. Thus, in some embodiments, the disclosure relates to AAV-mediated gene replacement therapy of NEU1 for patients having Alzheimer's Disease.

### Isolated Nucleic Acids

In some aspects, the disclosure provides a nucleic acid comprising at least one transgene operably linked to a promoter, wherein the transgene encodes NEU1. In some embodiments, the NEU1 gene comprises a nucleotide sequence set forth in any one of SEQ ID NOs: 1-4. In some embodiments, the NEU1 gene comprises a nucleotide sequence encoding an amino acid sequence as set forth in any one of SEQ ID NOs: 5-8.

The NEU1 gene encodes neuraminidase 1, a protein that catalyzes the removal of sialic acid (N-acetylneuraminic acid) moieties from glycoproteins and glycolipids. Active NEU1 protein comprises a 47-amino acid signal peptide.

The NEU1 gene may encode an mRNA having the nucleotide sequence of NM_000434.3. The NEU1 gene may encode a protein having the amino acid sequence of NP_000425.1. In some embodiments, the NEU1 transgene comprises the nucleic acid sequence according to SEQ ID NO: 1. In some embodiments, the NEU1 transgene comprises a nucleic acid sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 1. In some embodiments, the NEU1 transgene encodes the amino acid sequence according to SEQ ID NO: 5. In some embodiments, the NEU1 protein encodes an amino acid sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 5.

The CTSA gene may encode an mRNA having the nucleotide sequence of NM_000308.4, NM_001127695.2, or NM_001167594.2. The CTSA gene may encode a protein having the amino acid sequence of NP_000299.3, NP_001121167.1, or NP_001161066.1. In some embodiments, the CTSA transgene comprises the sequence according to SEQ ID NO: 9. In some embodiments, the CTSA transgene comprises a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 9. In some embodiments, the CTSA transgene encodes the amino acid sequence according to SEQ ID NO: 11. In some embodiments, the cathepsin A protein comprises an amino acid sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 11.

In some embodiments, a nucleic acid comprises a sequence encoding a signal peptide. In some embodiments, the sequence encoding a signal peptide is operably linked (e.g., adjacent to) a sequence encoding a signal peptide. In some embodiments, a nucleic acid encoding a NEU1 gene further comprises a signal peptide. In some embodiments, a nucleic acid encoding a CTSA gene further comprises a signal peptide.

In some embodiments, a signal peptide is a native or endogenous signal peptide. For example, in some embodiments, a signal peptide is a native or endogenous NEU1 signal peptide (e.g., comprising an amino acid sequence set forth in SEQ ID NO: 16). In some embodiments, a signal peptide is a variant of a native signal peptide. In some embodiments, a signal peptide directs a gene to a specific cellular location. In some embodiments, a signal peptide directs a gene to the lysosome. In some embodiments, a signal peptide is a signal peptide derived from a lysosomal protein. In some embodiments, a signal peptide is an iduronidase (IDUA) signal peptide (e.g., comprising an amino acid sequence set forth in SEQ ID NO: 18). In some embodiments, a signal peptide is a CTSA signal peptide (e.g., comprising an amino acid sequence set forth in SEQ ID NO: 17).

The terms “percent identity,” “sequence identity,” “% identity,” “% sequence identity,” and % identical,” as they may be interchangeably used herein, refer to a quantitative measurement of the similarity between two sequences (e.g., nucleic acid or amino acid). The percent identity of genomic DNA sequence, intron and exon sequence, and amino acid sequence between humans and other species varies by species type, with chimpanzee having the highest percent identity with humans of all species in each category. Percent identity can be determined using the algorithms of Karlin and Altschul, Proc. Natl. Acad. Sci. USA 87:2264-68, 1990, modified as in Karlin and Altschul, Proc. Natl. Acad. Sci. USA 90:5873-77, 1993. Such algorithms is incorporated into the NBLAST and XBLAST programs (version 2.0) of Altschul et al., J. Mol. Biol. 215:403-10, 1990. BLAST protein searches can be performed with the XBLAST program, score=50, word length=3, to obtain amino acid sequences homologous to the protein molecules of interest. Where gaps exist between two sequences, Gapped BLAST can be utilized as described in Altschul et al., Nucleic Acids Res. 25(17):3389-3402, 1997. When utilizing BLAST and Gapped BLAST programs, the default parameters of the respective programs (e.g., XBLAST and NBLAST) can be used. When a percent identity is stated, or a range thereof (e.g., at least, more than, etc.), unless otherwise specified, the endpoints shall be inclusive and the range (e.g., at least 70% identity) shall include all ranges within the cited range (e.g., at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity) and all increments thereof (e.g., tenths of a percent (i.e., 0.1%), hundredths of a percent (i.e., 0.01%), etc.).

In some aspects, the disclosure provides a nucleic acid comprising at least one transgene operably linked to a promoter, wherein the transgene encodes NEU1 and/or CTSA.

A transgene which encodes a protein is generally operably linked to a promoter. As used herein, “operably linked” sequences include both expression control sequences that are contiguous with the gene of interest (e.g., transgene) and expression control sequences that act in trans or at a distance to control the gene of interest (e.g., transgene). In some embodiments, the promoter is a constitutive promoter, for example a chicken beta-actin (CBA) promoter, a retroviral Rous sarcoma virus (RSV) long terminal repeat (LTR) promoter (optionally with the RSV enhancer), a cytomegalovirus (CMV) promoter (optionally with the CMV enhancer) [see, e.g., Boshart et al., Cell, 41:521-530 (1985)], a Simian vacuolating virus 40 (SV40) promoter, a dihydrofolate reductase promoter, a beta-actin promoter, a phosphoglycerol kinase (PGK) promoter, and a EF1alpha promoter [Invitrogen]. In some embodiments, a promoter is an enhanced chicken beta-actin promoter. In some embodiments, a promoter is a U6 promoter. In some embodiments, a promoter is an inducible promoter. Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only. In another embodiment, the native promoter for the transgene (e.g., NEU1, CTSA) will be used. The native promoter may be preferred when it is desired that expression of the transgene should mimic the native expression. The native promoter may be used when expression of the transgene must be regulated temporally or developmentally, or in a tissue-specific manner, or in response to specific transcriptional stimuli. In a further embodiment, other native expression control elements, such as enhancer elements, polyadenylation sites or Kozak consensus sequences may also be used to mimic the native expression.

In some embodiments, the promoter drives transgene expression in neuronal tissues. In some embodiments, the disclosure provides a nucleic acid operably comprising a tissue-specific promoter operably linked to a transgene. As used herein, “tissue-specific promoter” refers to a promoter that preferentially regulates (e.g., drives or up-regulates) gene expression in a particular cell type relative to other cell types. A cell-type-specific promoter can be specific for any cell type, such as liver cells (e.g., hepatocytes), heart cells, muscle cells, etc.

Further examples of tissue-specific promoters include but are not limited to a liver-specific thyroxin binding globulin (TBG) promoter, an insulin promoter, a creatine kinase (MCK) promoter, an alpha-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998), and the immunoglobulin heavy chain promoter, among others which will be apparent to the skilled artisan.

In some aspects, the disclosure relates to isolated nucleic acids comprising a transgene (e.g., NEU1, CTSA) operably linked to a promoter via a chimeric intron. In some embodiments, a chimeric intron comprises a nucleic acid sequence from a chicken beta-actin gene, for example a non-coding intronic sequence from intron 1 of the chicken beta-actin gene. In some embodiments, the intronic sequence of the chicken beta-actin gene ranges from about 50 to about 150 nucleotides in length (e.g., any length between 50 and 150 nucleotides, inclusive). In some embodiments, the intronic sequence of the chicken beta-actin gene ranges from about 100 to 120 (e.g., 100, 101, 102, 103, 104, 105, 106, 107, 108, 109, 110, 111, 112, 113, 114, 115, 116, 117, 118, 119, or 120) nucleotides in length. In some embodiments, a chimeric intron is adjacent to one or more untranslated sequences (e.g., an untranslated sequence located between the promoter sequence and the chimeric intron sequence and/or an untranslated sequence located between the chimeric intron and the first codon of the transgene sequence). In some embodiments, each of the one or more untranslated sequences are non-coding sequences from a rabbit beta-globulin gene (e.g., untranslated sequence from rabbit beta-globulin exon 1, exon 2, etc.).

Aspects of the disclosure relate to codon-optimized nucleic acid sequences. In some embodiments, the nucleic acid sequence encoding NEU1 protein and/or cathepsin A protein is a codon-optimized sequence (e.g., codon optimized for expression in mammalian cells). Without wishing to be bound by any particular theory, codon-optimization enables the reduction of certain undesirable characteristics in nucleic acid sequences, for example structural elements that may be immunogenic in a mammalian host (e.g., CpG islands, high GC content, etc.). In some embodiments, a codon-optimized sequence encoding NEU1 protein and/or cathepsin A protein comprises reduced GC content relative to a wild-type sequence that has not been codon-optimized. In some embodiments, a codon-optimized sequence encoding NEU1 protein and/or cathepsin A protein comprises a 1-5%, 3-5%, 3-10%, 5-10%, 5-15%, 10-20%, 15-30%, 20-40%, 25-50%, or 30-60% reduction in GC content relative to a wild-type sequence that has not been codon-optimized. In some embodiments, a codon-optimized sequence encoding NEU1 protein and/or cathepsin A protein comprises fewer guanine and/or cytosine nucleobases relative to a wild-type sequence that has not been codon-optimized. In some embodiments, a codon-optimized sequence encoding NEU1 protein and/or cathepsin A protein comprises 1-5, 3-5, 3-10, 5-10, 5-15, 10-20, 15-30, 20-40, 25-50, or 30-60 fewer guanine and/or cytosine nucleobases relative to a wild-type sequence that has not been codon-optimized. In some embodiments, a codon-optimized sequence encoding NEU1 protein and/or cathepsin A protein comprises fewer CpG dinucleotide islands relative to a wild-type sequence that has not been codon-optimized. In some embodiments, a codon-optimized sequence encoding NEU1 protein and/or cathepsin A protein comprises 1-3, 3-5, 3-10, 5-10, 5-15, 10-20, 15-30, 20-40, 25-50, or 30-60 fewer CpG dinucleotide islands relative to a wild-type sequence that has not been codon-optimized.

The term “expression cassette”, as used herein, refers to a nucleic acid construct comprising nucleic acid elements sufficient for the expression of a gene product. Typically, an expression cassette comprises a nucleic acid encoding a gene product operatively linked to a promoter sequence. The term “operatively linked” refers to the association of two or more nucleic acid fragments on a single nucleic acid fragment so that the function of one is affected by the other. For example, a promoter is operatively linked with a coding sequence when it is capable of affecting the expression of that coding sequence (e.g., the coding sequence is under the transcriptional control of the promoter). Encoding sequences can be operatively linked to regulatory sequences in sense or antisense orientation. In some embodiments, the promoter is a heterologous promoter. The term “heterologous promoter”, as used herein, refers to a promoter that does is not found to be operatively linked to a given encoding sequence in nature. In some embodiments, an expression cassette may comprise additional elements, for example, an intron, an enhancer, a polyadenylation site, a woodchuck response element (WRE), and/or other elements known to affect expression levels of the encoding sequence. Without wishing to be bound by theory, inclusion of an intron in an expression cassette, for example, between the transcriptional start site and an encoding nucleic acid sequence, for example, a protein-encoding cDNA sequence, is believed to result in increased expression levels of the encoding nucleic acid and the encoded gene product as compared to an expression construct not including an intron.

The term “intron” is art recognized and refers to a nucleic acid sequence in an expression cassette that is removed after transcription of a primary transcript by a cellular process termed splicing. Intron sequences generally comprise a splice donor and a splice acceptor and sequences of such donor and acceptor sites are well known to those of skill in the art. The term “positioned within an intron”, as used herein, refers to a nucleic acid construct, for example, an expression cassette, that is positioned between a splice donor and a splice acceptor sites of an intronic sequence.

In different embodiments, multicistronic expression constructs are provided in which the expression cassettes are positioned in different ways. For example, in some embodiments, a multicistronic expression construct is provided in which a first expression cassette is positioned adjacent to a second expression cassette. In some embodiments, a multicistronic expression construct is provided in which a first expression cassette comprises an intron, and a second expression cassette is positioned within the intron of the first expression cassette. In some embodiments, the second expression cassette, positioned within an intron of the first expression cassette, comprises a promoter and a nucleic acid sequence encoding a gene product operatively linked to the promoter.

In different embodiments, multicistronic expression constructs are provided in which the expression cassettes are oriented in different ways. For example, in some embodiments, a multicistronic expression construct is provided in which a first expression cassette is in the same orientation as a second expression cassette. In some embodiments, a multicistronic expression construct is provided comprising a first and a second expression cassette in opposite orientations.

The term “orientation” as used herein in connection with expression cassettes, refers to the directional characteristic of a given cassette or structure. In some embodiments, an expression cassette harbors a promoter 5′ of the encoding nucleic acid sequence, and transcription of the encoding nucleic acid sequence runs from the 5′ terminus to the 3′ terminus of the sense strand, making it a directional cassette (e.g. 5′-promoter/(intron)/encoding sequence-3′). Since virtually all expression cassettes are directional in this sense, those of skill in the art can easily determine the orientation of a given expression cassette in relation to a second nucleic acid structure, for example, a second expression cassette, a viral genome, or, if the cassette is comprised in an AAV construct, in relation to an AAV ITR. In some embodiments, one or more bindings sites for one or more of miRNAs are incorporated in a rAAV vector or nucleic acid (e.g., in a transgene), to inhibit the expression of the transgene in one or more tissues of an subject harboring the transgene. The skilled artisan will appreciate that binding sites may be selected to control the expression of a transgene in a tissue specific manner. For example, binding sites for the liver-specific miR-122 may be incorporated into a transgene to inhibit expression of that transgene in the liver. The target sites in the mRNA may be in the 5′ UTR, the 3′ UTR or in the coding region. Typically, the target site is in the 3′ UTR of the mRNA. Furthermore, the transgene may be designed such that multiple miRNAs regulate the mRNA by recognizing the same or multiple sites. The presence of multiple miRNA binding sites may result in the cooperative action of multiple RISCs and provide highly efficient inhibition of expression. The target site sequence may comprise a total of 5-100, 10-60, or more nucleotides. The target site sequence may comprise at least 5 nucleotides of the sequence of a target gene binding site.

### Recombinant AAVs

The isolated nucleic acids of the disclosure may be recombinant Adeno-associated viral (rAAVs) vectors. In some embodiments, an isolated nucleic acid as described by the disclosure comprises a region (e.g., a first region) comprising a first adeno-associated virus (AAV) inverted terminal repeat (ITR), or a variant thereof and a second region comprising a first transgene encoding NEU1. In some embodiments, the second region comprises a first transgene encoding NEU1. In some embodiments, the second region comprises a first transgene encoding CTSA. In some embodiments, the isolated nucleic acid further comprises a third region comprising a second transgene, which may or may not be different than the first transgene. In some embodiments, the second region comprises a first transgene encoding NEU1 and a third region comprises a second transgene encoding a second gene of interest (e.g., transgene). In some embodiments, the second region comprises a first transgene encoding CTSA and a third region comprises a second transgene encoding a second gene of interest (e.g., transgene). In some embodiments, the second region and third region each comprise the transgene encoding NEU1. In some embodiments, the second region and third region each comprise the transgene encoding CTSA. In some embodiments, the second region comprises a first transgene encoding NEU1 and a third region comprises a second transgene encoding CTSA. In some embodiments, the second region comprises a first transgene encoding CTSA and a third region comprises a second transgene encoding NEU1. The isolated nucleic acid (e.g., the rAAV vector) may be packaged into a capsid protein and administered to a subject and/or delivered to a selected target cell. The transgene may also comprise a region encoding, for example, a protein and/or an expression control sequence (e.g., a poly-A tail), as described elsewhere in the disclosure.

The instant disclosure provides a vector comprising a single, cis-acting WT ITR. In some embodiments, the ITR is a 5′ ITR. In some embodiments, the ITR is a 3′ ITR. Generally, ITR sequences are about 145 bp in length. Preferably, substantially the entire sequences encoding the ITR(s) is used in the molecule, although some degree of minor modification of these sequences is permissible.

In some embodiments, an rAAV vector or rAAV particle comprises a mutant ITR that lacks a functional terminal resolution site (TRS). The term “lacking a terminal resolution site” can refer to an AAV ITR that comprises a mutation (e.g., a sense mutation such as a non-synonymous mutation, or missense mutation) that abrogates the function of the terminal resolution site (TRS) of the ITR, or to a truncated AAV ITR that lacks a nucleic acid sequence encoding a functional TRS (e.g., a ΔTRS ITR). Without wishing to be bound by any particular theory, a rAAV vector comprising an ITR lacking a functional TRS produces a self-complementary rAAV vector, for example as described by McCarthy (2008) Molecular Therapy 16(10):1648-1656.

Another example of such a molecule employed in the present disclosure is a “cis-acting” plasmid containing the transgene, in which the selected transgene sequence and associated regulatory elements are flanked by the 5′ AAV ITR sequence and a 3′ hairpin-forming RNA sequence. Adeno-associated viral ITR sequences may be obtained from any known AAV, including presently identified mammalian AAV types. In some embodiments, an ITR sequence is an AAV2, AAV3, AAV4, AAV5, AAV6, AAV7, AAV8, AAVrh8, AAV9, AAV10, and/or AAVrh10 ITR sequence.

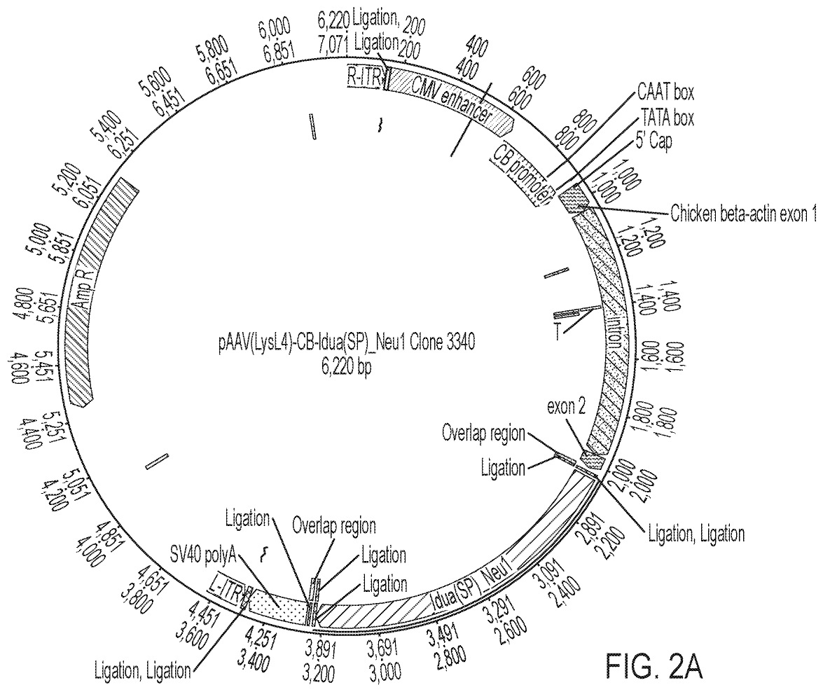

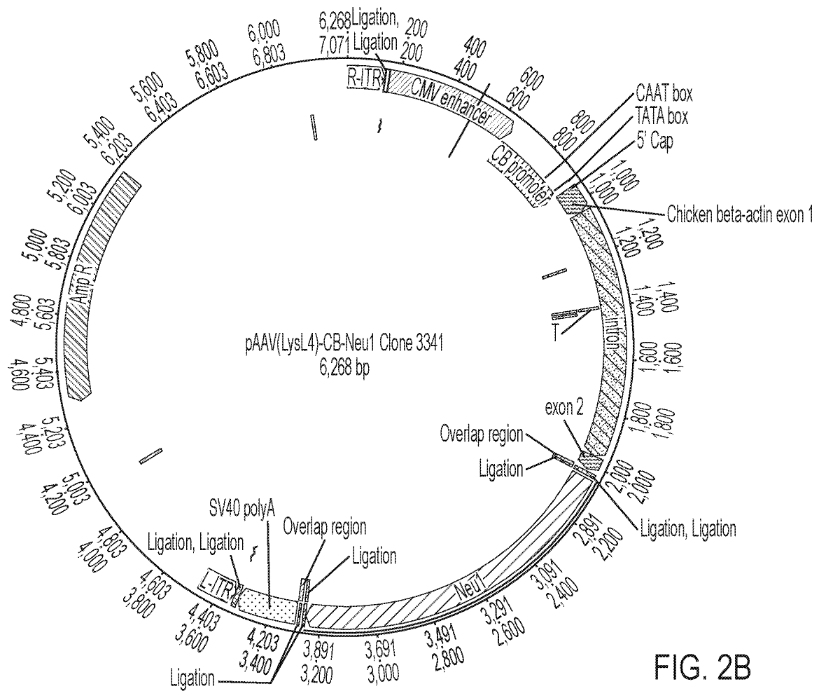

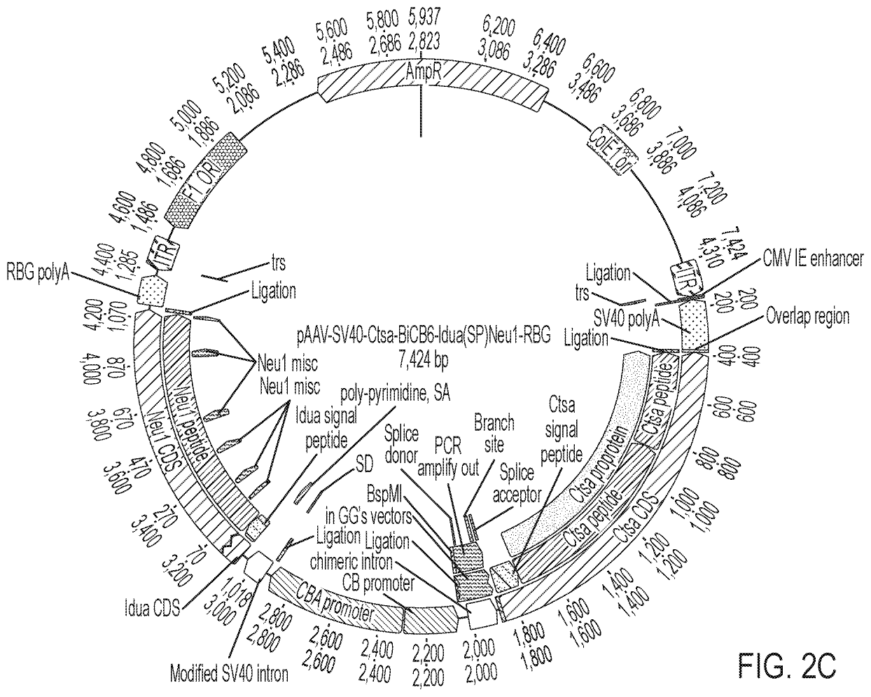

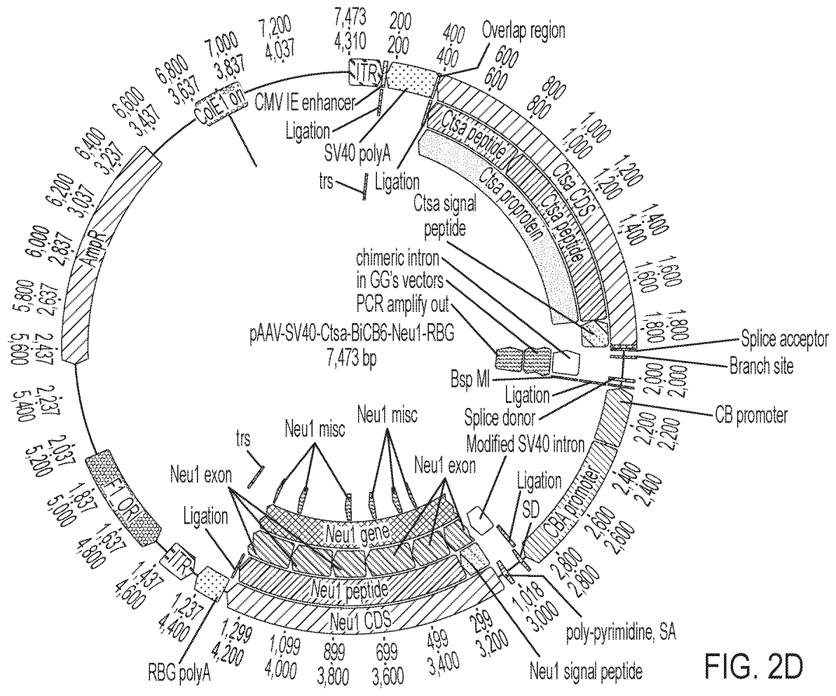

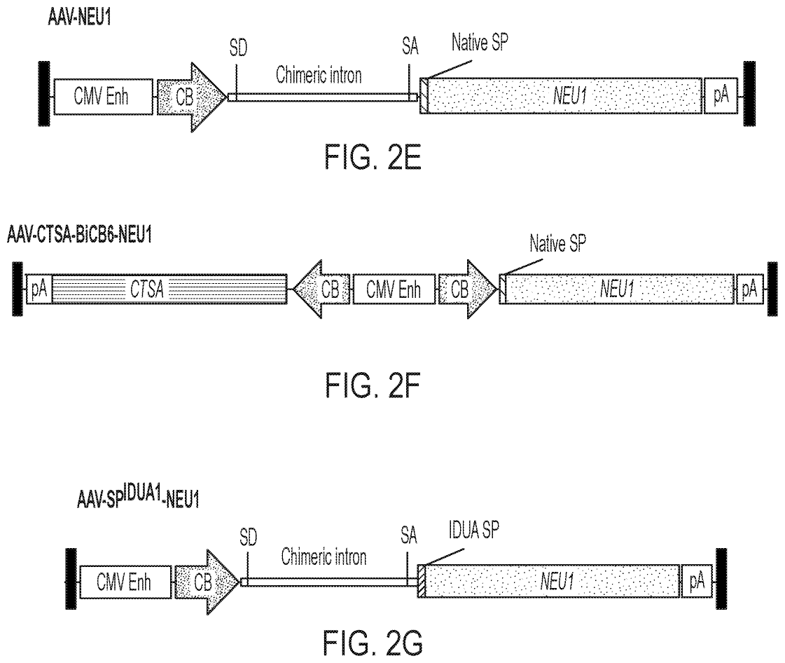

In some embodiments, an rAAV vector (e.g., as shown in FIGS. 2A-2D) comprises a nucleic acid sequence according to any one of SEQ ID NOs: 21-24, or a portion thereof. In some embodiments, the rAAV vector comprises a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to any one of SEQ ID NOs: 21-24.

In some embodiments, an rAAV vector is a self-complementary vector that comprises a nucleic acid sequence according to any one of SEQ ID NOs: 21-24, or a portion thereof. In some embodiments, the rAAV vector comprises a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to any one of SEQ ID NOs: 21-24.

The isolated nucleic acids and/or rAAVs of the present disclosure may be modified and/or selected to enhance the targeting of the isolated nucleic acids and/or rAAVs to a target tissue (e.g., liver or skeletal muscle). Non-limiting methods of modifications and/or selections include AAV capsid serotypes (e.g., AAV9), tissue-specific promoters, and/or targeting peptides. In some embodiments, the isolated nucleic acids and rAAVs of the present disclosure comprise AAV capsid serotypes with enhanced targeting to liver or skeletal muscle tissues (e.g., AAV9). In some embodiments, the isolated nucleic acids and rAAVs of the present disclosure comprise tissue-specific promoters. In some embodiments, the isolated nucleic acids and rAAVs of the present disclosure comprise AAV capsid serotypes with enhanced targeting to liver or skeletal muscle tissues and tissue-specific promoters.

In some aspects, the disclosure provides isolated AAVs. As used herein with respect to AAVs, the term “isolated” refers to an AAV that has been artificially obtained or produced. Isolated AAVs may be produced using recombinant methods. Such AAVs are referred to herein as “recombinant AAVs” or “rAAVs.” Recombinant AAVs preferably have tissue-specific targeting capabilities, such that a transgene of the rAAV will be delivered specifically to one or more predetermined tissue(s). The AAV capsid is an important element in determining these tissue-specific targeting capabilities. Thus, an rAAV having a capsid appropriate for the tissue being targeted can be selected. In some embodiments, the rAAV comprises an AAV1, AAV2, AAV3, AAV4, AAV5, AAV6, AAV7, AAV8, AAVrh8, AAV9, AAV10 or AAVrh10 capsid protein, or a protein having substantial homology thereto. In some embodiments, the rAAV comprises an AAV9 capsid protein.

In some embodiments, the rAAVs of the disclosure are pseudo-typed rAAVs. Pseudo-typing is the process of producing viruses or viral vectors in combination with foreign viral envelope proteins. The result is a pseudo-typed virus particle. With this method, the foreign viral envelope proteins can be used to alter host tropism or an increased/decreased stability of the virus particles. In some aspects, a pseudo-typed rAAV comprises nucleic acids from two or more different AAVs, wherein the nucleic acid from one AAV encodes a capsid protein and the nucleic acid of at least one other AAV encodes other viral proteins and/or the viral genome. In some embodiments, a pseudo-typed rAAV refers to an AAV comprising an inverted terminal repeats (ITRs) of one AAV serotype and an capsid protein of a different AAV serotype. For example, a pseudo-typed AAV vector containing the ITRs of serotype X encapsidated with the proteins of Y will be designated as AAVX/Y (e.g., AAV2/1 has the ITRs of AAV2 and the capsid of AAV1). In some embodiments, pseudo-typed rAAVs may be useful for combining the tissue-specific targeting capabilities of a capsid protein from one AAV serotype with the viral DNA from another AAV serotype, thereby allowing targeted delivery of a transgene to a target tissue.

Methods for obtaining rAAVs having a desired capsid protein are well known in the art. (See, for example, US Patent Application Publication Number US 2003/0138772, the contents of which are incorporated herein by reference in their entirety). Typically, the methods involve culturing a host cell which contains a nucleic acid sequence encoding an AAV capsid protein or fragment thereof; a functional rep gene; an rAAV vector composed of, AAV inverted terminal repeats (ITRs) and a transgene; and sufficient helper functions to permit packaging of the rAAV vector into the AAV capsid proteins. Typically, capsid proteins are structural proteins encoded by the cap gene of an AAV. In some embodiments, AAVs comprise three capsid proteins, virion proteins 1 to 3 (named VP1, VP2 and VP3), all of which are transcribed from a single cap gene via alternative splicing. In some embodiments, the molecular weights of VP1, VP2 and VP3 are respectively about 87 kDa, about 72 kDa and about 62 kDa. In some embodiments, upon translation, capsid proteins form a spherical 60-mer protein shell around the viral genome. In some embodiments, capsid proteins protect a viral genome, deliver a genome and/or interact with a host cell. In some aspects, capsid proteins deliver the viral genome to a host in a tissue specific manner.

In some embodiments, the AAV capsid protein is of an AAV serotype selected from the group consisting of AAV3, AAV4, AAV5, AAV6, AAV8, AAVrh8 AAV9, AAV10 and AAVrh10. In some embodiments, the AAV capsid protein is of an AAVrh8 or AAVrh10 serotype. In some embodiments, the AAV capsid protein is of an AAVrh8 serotype.

In some embodiments, components to be cultured in the host cell to package an rAAV vector in an AAV capsid may be provided to the host cell in trans. Alternatively, any one or more of the required components (e.g., rAAV vector, rep sequences, cap sequences, and/or helper functions) may be provided by a stable host cell which has been engineered to contain one or more of the required components using methods known to those of skill in the art. Most suitably, such a stable host cell will contain the required component(s) under the control of an inducible promoter. However, the required component(s) may be under the control of a constitutive promoter. Examples of suitable inducible and constitutive promoters are provided herein, in the discussion of regulatory elements suitable for use with the transgene. In still another alternative, a selected stable host cell may contain selected component(s) under the control of a constitutive promoter and other selected component(s) under the control of one or more inducible promoters. For example, a stable host cell may be generated which is derived from 293 cells (which contain E1 helper functions under the control of a constitutive promoter), but which contain the rep and/or cap proteins under the control of inducible promoters. Still other stable host cells may be generated by one of skill in the art.

In some embodiments, the disclosure relates to a host cell containing a nucleic acid that comprises a coding sequence selected from the group consisting of any one of SEQ ID NOs: 1-4 and/or SEQ ID NO 9 or 10 that is operably linked to a promoter. In some embodiments, the disclosure relates to a host cell containing a nucleic acid comprising a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 1-8 operably linked to a promoter. In some embodiments, the disclosure relates to a host cell containing a nucleic acid that comprises a coding sequence selected from the group consisting of any one of SEQ ID NOs: 1-4 and/or SEQ ID NO 9 or 10 that is operably linked to a promoter. In some embodiments, the disclosure relates to a host cell containing a nucleic acid comprising a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to SEQ ID NO: 1 operably linked to a promoter. In some embodiments, the disclosure relates to a host cell containing a nucleic acid that comprises a coding sequence selected from the group consisting of any one of SEQ ID NOs: 1-4 and/or SEQ ID NO 9 or 10 that is operably linked to a promoter. In some embodiments, the disclosure relates to a host cell containing a nucleic acid comprising a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to any one of SEQ ID NOs: 1-4 and/or SEQ ID NO 9 or 10 operably linked to a promoter. In some embodiments, the disclosure relates to a host cell containing a nucleic acid that comprises a coding sequence selected from the group consisting of any one of SEQ ID NOs: 1-4 and/or SEQ ID NO 9 or 10 that is operably linked to a promoter. In some embodiments, the disclosure relates to a host cell containing a nucleic acid comprising a sequence having at least 80% identity (e.g., at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 95.5%, at least 96%, at least 96.5%, at least 97%, at least 97.5%, at least 98%, at least 98.5%, at least 99%, at least 99.5%, at least 99.6%, at least 99.7%, at least 99.8%, at least 99.9% identity or more) to any one of SEQ ID NOs: 1-4 and/or SEQ ID NO 9 or 10 operably linked to a promoter.

In some embodiments, the disclosure relates to a composition comprising the host cell described above. In some embodiments, the composition comprising the host cell above further comprises a cryopreservative.

The rAAV vector, rep sequences, cap sequences, and helper functions useful for producing the rAAV of the disclosure may be delivered to the packaging host cell using any appropriate genetic element (vector). The selected genetic element may be delivered by any suitable method, including those described herein. The methods used to construct any embodiment of this disclosure are known to those with skill in nucleic acid manipulation and include genetic engineering, recombinant engineering, and synthetic techniques. See, e.g., Sambrook et al., Molecular Cloning: A Laboratory Manual, Cold Spring Harbor Press, Cold Spring Harbor, N.Y. Similarly, methods of generating rAAV virions (i.e., infectious viral particle) are well known and the selection of a suitable method is not a limitation on the present disclosure. See, e.g., K. Fisher et al., J. Virol., 70:520-532 (1993) and U.S. Pat. No. 5,478,745.

In some embodiments, rAAVs may be produced using the triple transfection method (described in detail in U.S. Pat. No. 6,001,650). Typically, the rAAVs are produced by transfecting a host cell with an rAAV vector (comprising a transgene) to be packaged into AAV particles, an AAV helper function vector, and an accessory function vector. An AAV helper function vector encodes the “AAV helper function” sequences (i.e., rep and cap), which function in trans for productive AAV replication and encapsidation. Preferably, the AAV helper function vector supports efficient AAV vector production without generating any detectable WT AAV virions (i.e., AAV virions containing functional rep and cap genes). Non-limiting examples of vectors suitable for use with the present disclosure include pHLP19, described in U.S. Pat. No. 6,001,650 and pRep6cap6 vector, described in U.S. Pat. No. 6,156,303, the entirety of both incorporated by reference herein. The accessory function vector encodes nucleotide sequences for non-AAV derived viral and/or cellular functions upon which AAV is dependent for replication (i.e., “accessory functions”). The accessory functions include those functions required for AAV replication, including, without limitation, those moieties involved in activation of AAV gene transcription, stage specific AAV mRNA splicing, AAV DNA replication, synthesis of cap expression products, and AAV capsid assembly. Viral-based accessory functions can be derived from any of the known helper viruses such as adenovirus, herpesvirus (other than herpes simplex virus type-1), and vaccinia virus.

In some aspects, the disclosure provides transfected host cells. The term “transfection” is used to refer to the uptake of foreign DNA by a cell, and a cell has been “transfected” when exogenous DNA has been introduced through the cell membrane. A number of transfection techniques are generally known in the art. See, e.g., Graham et al. (1973) Virology, 52:456, Sambrook et al. (1989) Molecular Cloning, a laboratory manual, Cold Spring Harbor Laboratories, New York, Davis et al. (1986) Basic Methods in Molecular Biology, Elsevier, and Chu et al. (1981) Gene 13:197. Such techniques can be used to introduce one or more exogenous nucleic acids, such as a nucleotide integration vector and other nucleic acid molecules, into suitable host cells.

A “host cell” refers to any cell that harbors, or is capable of harboring, a substance of interest. Often a host cell is a mammalian cell. A host cell may be used as a recipient of an AAV helper construct, an AAV minigene plasmid, an accessory function vector, or other transfer DNA associated with the production of rAAVs. The term includes the progeny of the original cell which has been transfected. Thus, a “host cell” as used herein may refer to a cell which has been transfected with an exogenous DNA sequence. It is understood that the progeny of a single parental cell may not necessarily be completely identical in morphology or in genomic or total DNA complement as the original parent, due to natural, accidental, or deliberate mutation.

As used herein, the term “cell line” refers to a population of cells capable of continuous or prolonged growth and division in vitro. Often, cell lines are clonal populations derived from a single progenitor cell. It is further known in the art that spontaneous or induced changes can occur in karyotype during storage or transfer of such clonal populations. Therefore, cells derived from the cell line referred to may not be precisely identical to the ancestral cells or cultures, and the cell line referred to includes such variants.

As used herein, the term “recombinant cell” refers to a cell into which an exogenous DNA segment, such as DNA segment that leads to the transcription of a biologically-active polypeptide or production of a biologically active nucleic acid such as an RNA, has been introduced.

As used herein, the term “vector” includes any genetic element, such as a plasmid, phage, transposon, cosmid, chromosome, artificial chromosome, virus, virion, etc., which is capable of replication when associated with the proper control elements and which can transfer gene sequences between cells. Thus, the term includes cloning and expression vehicles, as well as viral vectors. In some embodiments, useful vectors are contemplated to be those vectors in which the nucleic acid segment to be transcribed is positioned under the transcriptional control of a promoter. A “promoter” refers to a DNA sequence recognized by the synthetic machinery of the cell, or introduced synthetic machinery, required to initiate the specific transcription of a gene. The phrases “operatively positioned,” “under control,” or “under transcriptional control” means that the promoter is in the correct location and orientation in relation to the nucleic acid to control RNA polymerase initiation and expression of the gene. The term “expression vector or construct” means any type of genetic construct containing a nucleic acid in which part or all of the nucleic acid encoding sequence is capable of being transcribed. In some embodiments, expression includes transcription of the nucleic acid, for example, to generate a biologically-active polypeptide product or inhibitory RNA (e.g., shRNA, miRNA, miRNA inhibitor) from a transcribed gene.

The foregoing methods for packaging recombinant vectors in desired AAV capsids to produce the rAAVs of the disclosure are not meant to be limiting and other suitable methods will be apparent to the skilled artisan.

The isolated nucleic acids of the present disclosure may be rAAV vectors. Recombinant AAV vectors of the disclosure are typically composed of, at a minimum, a transgene and its regulatory sequences, and 5′ and 3′ AAV ITRs. It is this rAAV vector which is packaged into a capsid protein and delivered to a selected target cell. In some embodiments, the transgene is a nucleic acid sequence, heterologous to the vector sequences, which encodes a polypeptide, protein, functional RNA molecule or other gene product, of interest. The nucleic acid coding sequence is operatively linked to regulatory components in a manner which permits transgene transcription, translation, and/or expression in a cell of a target tissue.

Aspects of the disclosure relate to the discovery that modifying the regulatory sequences of rAAVs provides levels of transgene expression that are therapeutically effective yet do not cause the vector-mediated toxicity associated with previously used rAAVs. Accordingly, in some embodiments, the disclosure relates to an rAAV comprising modified genetic regulatory elements. In some embodiments, the modified genetic regulatory element is a hybrid promoter. As used herein, the term “hybrid promoter” refers to a regulatory construct capable of driving transcription an RNA transcript (e.g., a transcript comprising encoded by a transgene) in which the construct comprises two or more regulatory elements artificially arranged. Typically, a hybrid promoter comprises at least one element that is a minimal promoter and at least one element having an enhancer sequence or an intronic, exonic, or untranslated region (UTR) sequence comprising one or more transcriptional regulatory elements. In embodiments in which a hybrid promoter comprises an exonic, intronic, or UTR sequence, such sequence(s) may encode upstream portions of the RNA transcript while also containing regulatory elements that modulate (e.g., enhance) transcription of the transcript. In some embodiments, two or more elements of a hybrid promoter are from heterologous sources relative to one another. In some embodiments, two or more elements of a hybrid promoter are from heterologous sources relative to the transgene. In some embodiments, two or more elements of a hybrid promoter are from different genetic loci. In some embodiments, two or more elements of a hybrid promoter are from the same genetic locus but are arranged in a manner not found at the genetic locus. In some embodiments, the hybrid promoter comprises a first nucleic acid sequence from one promoter fused to one or more nucleic acid sequences comprises promoter or enhancer elements of a difference source. In some embodiments, a hybrid promoter comprises a first sequence from the chicken beta-actin promoter and a second sequence of the CMV enhancer. In some embodiments, a hybrid promoter comprises a first sequence from a chicken beta-actin promoter and a second sequence from an intron of a chicken-beta actin gene. In some embodiments, a hybrid promoter comprises a first sequence from the chicken beta-actin promoter fused to a CMV enhancer sequence and a sequence from an intron of the chicken-beta actin gene.

In some aspects, the rAAV comprises an enhancer element. As used herein, the term “enhancer element” refers to a nucleic acid sequence that when bound by an activator protein, activates or increases transcription of a gene or genes. Enhancer sequences can be upstream (i.e., 5′) or downstream (i.e., 3′) relative to the genes they regulate. Examples of enhancer sequences include cytomegalovirus (CMV) enhancer sequence and the SV40 enhancer sequence. In some embodiments, rAAVs comprise a CMV enhancer element or a portion thereof. As used herein, the term “a portion thereof” refers to a fragment of a nucleotide or amino acid sequence that retains the desired functional characteristic of the entire nucleotide or amino acid sequence from which it is derived. For example, a “CMV enhancer sequence or a portion thereof” refers to a nucleotide sequence derived from WT CMV enhancer that is capable of increasing transcription of a transgene.

In some aspects, the rAAV comprises a posttranscriptional response element. As used herein, the term “posttranscriptional response element” refers to a nucleic acid sequence that, when transcribed, adopts a tertiary structure that enhances expression of a gene. Examples of posttranscriptional regulatory elements include, but are not limited to, woodchuck hepatitis virus posttranscriptional regulatory element (WPRE), mouse RNA transport element (RTE), constitutive transport element (CTE) of the simian retrovirus type 1 (SRV-1), the CTE from the Mason-Pfizer monkey virus (MPMV), and the 5′ untranslated region of the human heat shock protein 70 (Hsp70 5′UTR). In some embodiments, the rAAV vector comprises a woodchuck hepatitis virus posttranscriptional regulatory element (WPRE).

In some aspects, the disclosure provides rAAV vectors comprising a hybrid or chimeric intron. As used herein, the term “chimeric intron” refers an intron having sequences from two or more different sources. In some embodiments, a chimeric intron comprises a nucleic acid encoding a splice donor site from a first source (e.g., organism or species) and a splice acceptor site from a second source (e.g., organism or species). In some embodiments, a chimeric intron comprise one or more transcriptional regulatory elements and/or enhancer sequences. In some embodiments, a chimeric intron is positioned between an exon of a hybrid promoter and transgene.

In some embodiments, the disclosure provides an rAAV comprising a promoter operably linked to a transgene, wherein the transgene encodes a NEU1 gene. In some embodiments, the disclosure provides an rAAV comprising a first expression cassette comprising a promoter operably linked to a transgene, wherein the transgene encodes a NEU1 gene, and wherein the rAAV further comprises a second expression cassette comprising a promoter operably linked to a transgene, wherein the transgene encodes a CTSA gene. In some embodiments, the disclosure provides an rAAV comprising a promoter operably linked to a first transgene, wherein the transgene encodes a NEU1 protein and a second transgene wherein the transgene encodes a Cathepsin A protein.

In certain embodiments, the disclosure relates to rAAV vectors comprising artificial transcription elements. As used here, the term “artificial transcription element” refers, in some embodiments, to a synthetic sequence enabling the controlled transcription of DNA by an RNA polymerase to produce an RNA transcript. Transcriptionally active elements of the present disclosure are generally smaller than 500 bp, preferably smaller than 200 bp, more preferably smaller than 100, most preferably smaller than 50 bp. In some embodiments, an artificial transcription element comprises two or more nucleic acid sequences from transcriptionally active elements. Transcriptionally active elements are generally recognized in the art and include, for example, promoter, enhancer sequence, TATA box, G/C box, CCAAT box, specificity protein 1 (Sp1) binding site, Inr region, CRE (cAMP regulatory element), activating transcription factor 1 (ATF1) binding site, ATF1-CRE binding site, APBbeta box, APBalpha box, CArG box, CCAC box and those disclosed by U.S. Pat. No. 6,346,415. Combinations of the foregoing transcriptionally active elements are also contemplated.

In some embodiments, the artificial transcription element comprises promoter sequence. In some embodiments, the artificial transcription element comprises enhancer sequence. In some embodiments, the artificial transcription element comprises ATF1-CRE binding site. In some embodiments, the artificial transcription element comprises SP1 binding site. In some embodiments, the artificial transcription element comprises C box. In some embodiments, the artificial transcription element comprises TATA box. In some embodiments, the artificial transcription element comprises ATF1-CRE binding site, SP1 binding site and TATA box. Expression control sequences include appropriate transcription initiation, termination, promoter and enhancer sequences; efficient RNA processing signals such as splicing and polyadenylation (polyA) signals; sequences that stabilize cytoplasmic mRNA; sequences that enhance translation efficiency (i.e., Kozak consensus sequence); sequences that enhance protein stability; and when desired, sequences that enhance secretion of the encoded product. A great number of expression control sequences, including promoters which are native, constitutive, inducible and/or tissue-specific, are known in the art and may be utilized.

As used herein, a nucleic acid sequence (e.g., coding sequence) and regulatory sequences are said to be “operably” linked when they are covalently linked in such a way as to place the expression or transcription of the nucleic acid sequence under the influence or control of the regulatory sequences. If it is desired that the nucleic acid sequences be translated into a functional protein, two DNA sequences are said to be operably linked if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region would be operably linked to a nucleic acid sequence if the promoter region were capable of effecting transcription of that DNA sequence such that the resulting transcript might be translated into the desired protein or polypeptide. Similarly two or more coding regions are operably linked when they are linked in such a way that their transcription from a common promoter results in the expression of two or more proteins having been translated in frame. In some embodiments, operably linked coding sequences yield a fusion protein. In some embodiments, operably linked coding sequences yield a functional RNA (e.g., shRNA, miRNA, miRNA inhibitor).

For nucleic acids encoding proteins, a polyadenylation sequence generally is inserted following the transgene sequences and before the 3′ AAV ITR sequence. An rAAV construct useful in the present disclosure may also contain an intron, desirably located between the promoter/enhancer sequence and the transgene. One possible intron sequence is derived from SV-40, and is referred to as the SV-40 T intron sequence.

Another vector element that may be used is an internal ribosome entry site (IRES). An IRES sequence is used to produce more than one polypeptide from a single gene transcript. An IRES sequence would be used to produce a protein that contain more than one polypeptide chains. Selection of these and other common vector elements are conventional and many such sequences are available [see, e.g., Sambrook et al., and references cited therein at, for example, pages 3.18 3.26 and 16.17 16.27 and Ausubel et al., Current Protocols in Molecular Biology, John Wiley & Sons, New York, 1989]. In some embodiments, a Foot and Mouth Disease Virus 2A sequence is included in polyprotein; this is a small peptide (approximately 18 amino acids in length) that has been shown to mediate the cleavage of polyproteins (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459).

The cleavage activity of the 2A sequence has previously been demonstrated in artificial systems including plasmids and gene therapy vectors (AAV and retroviruses) (Ryan, M D et al., EMBO, 1994; 4: 928-933; Mattion, N M et al., J Virology, November 1996; p. 8124-8127; Furler, S et al., Gene Therapy, 2001; 8: 864-873; and Halpin, C et al., The Plant Journal, 1999; 4: 453-459; de Felipe, P et al., Gene Therapy, 1999; 6: 198-208; de Felipe, P et al., Human Gene Therapy, 2000; 11: 1921-1931; and Klump, H et al., Gene Therapy, 2001; 8: 811-817).

The precise nature of the regulatory sequences needed for gene expression in host cells may vary between species, tissues or cell types, but shall in general include, as necessary, 5′ non-transcribed and 5′ non-translated sequences involved with the initiation of transcription and translation respectively, such as a TATA box, capping sequence, CAAT sequence, enhancer elements, and the like. Especially, such 5′ non-transcribed regulatory sequences will include a promoter region that includes a promoter sequence for transcriptional control of the operably joined gene. Regulatory sequences may also include enhancer sequences or upstream activator sequences as desired. The vectors of the disclosure may optionally include 5′ leader or signal sequences.

Examples of constitutive promoters include, without limitation, the retroviral Rous sarcoma virus (RSV) LTR promoter (optionally with the RSV enhancer), the cytomegalovirus (CMV) promoter (optionally with the CMV enhancer) [see, e.g., Boshart et al., Cell, 41:521-530 (1985)], the SV40 promoter, the dihydrofolate reductase promoter, the beta-actin promoter, the phosphoglycerol kinase (PGK) promoter, and the EF1alpha promoter [Invitrogen].

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech, and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system (WO 98/10088); the ecdysone insect promoter (No et al., Proc. Natl. Acad. Sci. USA, 93:3346-3351 (1996)), the tetracycline-repressible system (Gossen et al., Proc. Natl. Acad. Sci. USA, 89:5547-5551 (1992)), the tetracycline-inducible system (Gossen et al., Science, 268:1766-1769 (1995), see also Harvey et al., Curr. Opin. Chem. Biol., 2:512-518 (1998)), the RU486-inducible system (Wang et al., Nat. Biotech., 15:239-243 (1997) and Wang et al., Gene Ther., 4:432-441 (1997)) and the rapamycin-inducible system (Magari et al., J. Clin. Invest., 100:2865-2872 (1997)). Still other types of inducible promoters which may be useful in this context are those which are regulated by a specific physiological state, e.g., temperature, acute phase, a particular differentiation state of the cell, or in replicating cells only.

The AAV sequences of the vector typically comprise the cis-acting 5′ and 3′ inverted terminal repeat sequences (See, e.g., B. J. Carter, in “Handbook of Parvoviruses”, ed., P. Tijsser, CRC Press, pp. 155 168 (1990)). The ITR sequences are about 145 bp in length. Preferably, substantially the entire sequences encoding the ITRs are used in the molecule, although some degree of minor modification of these sequences is permissible. The ability to modify these ITR sequences is within the skill of the art. (See, e.g., texts such as Sambrook et al., “Molecular Cloning. A Laboratory Manual”, 2d ed., Cold Spring Harbor Laboratory, New York (1989); and K. Fisher et al., J Virol., 70:520 532 (1996)). An example of such a molecule employed in the present disclosure is a “cis-acting” plasmid containing the transgene, in which the selected transgene sequence and associated regulatory elements are flanked by the 5′ and 3′ AAV ITR sequences. The AAV ITR sequences may be obtained from any known AAV, including presently identified mammalian AAV types.

In some embodiments, the rAAVs of the disclosure are pseudo-typed rAAVs. For example, a pseudo-typed AAV vector containing the ITRs of serotype X encapsidated with the proteins of Y will be designated as AAVX/Y (e.g., AAV2/1 has the ITRs of AAV2 and the capsid of AAV1). In some embodiments, pseudo-typed rAAVs may be useful for combining the tissue-specific targeting capabilities of a capsid protein from one AAV serotype with the viral DNA from another AAV serotype, thereby allowing targeted delivery of a transgene to a target tissue.

In addition to the major elements identified above for the rAAV vector, the vector also includes conventional control elements necessary which are operably linked to the transgene in a manner which permits its transcription, translation and/or expression in a cell transfected with the plasmid vector or infected with the virus produced by the disclosure.

### Recombinant AAV Administration Methods

The rAAVs may be delivered to a subject in compositions according to any appropriate methods known in the art. The rAAV, preferably suspended in a physiologically compatible carrier (i.e., in a composition), may be administered to a subject (i.e., host animal (e.g., human, mouse, rat, cat, dog, sheep, rabbit, horse, cow, goat, pig, guinea pig, hamster, chicken, turkey, or a non-human primate (e.g., Macaque))). In some embodiments a host animal does not include a human.

Delivery of the rAAVs to a mammalian subject may be by, for example, intramuscular injection or by administration into the bloodstream of the mammalian subject. Administration into the bloodstream may be by injection into a vein, an artery, or any other vascular conduit. In some embodiments, the rAAVs are administered into the bloodstream by way of isolated limb perfusion, a technique well known in the surgical arts, the method essentially enabling the artisan to isolate a limb from the systemic circulation prior to administration of the rAAV virions. A variant of the isolated limb perfusion technique, described in U.S. Pat. No. 6,177,403, can also be employed by the skilled artisan to administer the virions into the vasculature of an isolated limb to potentially enhance transduction into muscle cells or tissue.

Aspects of the disclosure relate to compositions comprising an rAAV comprising at least one modified genetic regulatory sequence or element. In some embodiments, the composition further comprises a pharmaceutically acceptable carrier. The compositions of the disclosure may comprise an rAAV alone, or in combination with one or more other viruses (e.g., a second rAAV encoding having one or more different transgenes). In some embodiments, a composition comprises 1, 2, 3, 4, 5, 6, 7, 8, 9, 10, or more different rAAVs each having one or more different transgenes.

In some aspects, the disclosure relates to a composition (e.g., a pharmaceutical composition) comprising an rAAV comprising a nucleic acid encoding a NEU1 gene. In some aspects, the disclosure relates to a composition (e.g., a pharmaceutical composition) comprising an rAAV comprising a nucleic acid encoding a CTSA gene. In some aspects, the disclosure relates to a composition (e.g., a pharmaceutical composition) comprising an rAAV comprising a nucleic acid encoding NEU1 and CTSA genes.

Suitable carriers may be readily selected by one of skill in the art in view of the indication for which the rAAV is directed. For example, one suitable carrier includes saline, which may be formulated with a variety of buffering solutions (e.g., phosphate buffered saline). Other exemplary carriers include sterile saline, lactose, sucrose, calcium phosphate, gelatin, dextran, agar, pectin, peanut oil, sesame oil, and water. The selection of the carrier is not a limitation of the present disclosure.

Optionally, the compositions of the disclosure may contain, in addition to the rAAV and carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

Recombinant AAVs are administered in sufficient amounts to transfect the cells of a desired tissue and to provide sufficient levels of gene transfer and expression without undue adverse effects. Conventional and pharmaceutically acceptable routes of administration include, but are not limited to, direct delivery to the selected organ (e.g., injection into the liver, skeletal muscle), oral, inhalation (including intranasal and intratracheal delivery), intraocular, intravenous, intramuscular, subcutaneous, intradermal, intratumoral, and other parental routes of administration. Routes of administration may be combined, if desired.

The dose of rAAV virions required to achieve a particular “therapeutic effect,” e.g., the units of dose in genome copies/per kilogram of body weight (GC/kg), will vary based on several factors including, but not limited to: the route of rAAV virion administration, the level of gene or RNA expression required to achieve a therapeutic effect, the specific disease or disorder being treated, and the stability of the gene or RNA product. One of skill in the art can readily determine an rAAV virion dose range to treat a patient having a particular disease or disorder based on the aforementioned factors, as well as other factors that are well known in the art.

The terms “effective amount,” “therapeutically effective amount,” and “pharmaceutically effective amount,” as may be used interchangeably herein, refer to an amount of a biologically active agent (e.g., the isolated nucleic acids, rAAV, compositions of the present disclosure) sufficient to elicit a desired response. The effective amount will depend primarily on factors such as the species, age, weight, health of the subject, and the tissue to be targeted, and may thus vary among animal and tissue. For example, an effective amount of the rAAV is generally in the range from about 1 ml to about 100 ml of solution containing from about 10⁶ to 10¹⁶ genome copies (e.g., from 1×10⁶ to 1×10¹⁶, inclusive). In some cases, a dosage between about 10¹¹ to 10¹² rAAV genome copies is appropriate. In some embodiments, a dosage of between about 10¹¹ to 10¹³ rAAV genome copies is appropriate. In some embodiments, a dosage of between about 10¹¹ to 10¹⁴ rAAV genome copies is appropriate. In some embodiments, a dosage of between about 10¹¹ to 10¹⁵ rAAV genome copies is appropriate. In some embodiments, a dosage of 4.68×10⁷ is appropriate. In some embodiments, a dosage of 4.68×10⁸ genome copies is appropriate. In some embodiments, a dosage of 4.68×10⁹ genome copies is appropriate. In some embodiments, a dosage of 1.17×10¹⁰ genome copies is appropriate. In some embodiments, a dosage of 2.34×10¹⁰ genome copies is appropriate. In some embodiments, a dosage of 3.20×10¹¹ genome copies is appropriate. In some embodiments, a dosage of 1.2×10¹³ genome copies is appropriate. In some embodiments, a dosage of about 1×10¹⁴ vector genome (vg) copies is appropriate.

In some aspects, the disclosure relates to the recognition that one potential side-effect for administering an AAV to a subject is an immune response in the subject to the AAV, including inflammation. In some embodiments, a subject is immunosuppressed prior to administration of one or more rAAVs as described herein.

As used herein, “immunosuppressed” or “immunosuppression” refers to a decrease in the activation or efficacy of an immune response in a subject. Immunosuppression can be induced in a subject using one or more (e.g., multiple, such as 2, 3, 4, 5, or more) agents, including, but not limited to, rituximab, methylprednisolone, prednisolone, sirolimus, immunoglobulin injection, prednisone, methotrexate, and any combination thereof.

In some embodiments, methods described by disclosure further comprise the step inducing immunosuppression (e.g., administering one or more immunosuppressive agents) in a subject prior to the subject being administered an rAAV (e.g., an rAAV or pharmaceutical composition as described by the disclosure). In some embodiments, a subject is immunosuppressed (e.g., immunosuppression is induced in the subject) between about 30 days and about 0 days (e.g., any time between 30 days until administration of the rAAV, inclusive) prior to administration of the rAAV to the subject. In some embodiments, the subject is pre-treated with immune suppression (e.g., rituximab, sirolimus, and/or prednisone) for at least 7 days.

In some embodiments, immunosuppression of a subject maintained during and/or after administration of an rAAV or pharmaceutical composition. In some embodiments, a subject is immunosuppressed (e.g., administered one or more immunosuppressants) for between 1 day and 1 year after administration of the rAAV or pharmaceutical composition.

In some embodiments, rAAV compositions are formulated to reduce aggregation of AAV particles in the composition, particularly where high rAAV concentrations are present (e.g., ˜10¹³ GC/ml or more). Methods for reducing aggregation of rAAVs are well known in the art and, include, for example, addition of surfactants, pH adjustment, salt concentration adjustment, etc. See, e.g., Wright F R, et al., Molecular Therapy (2005) 12, 171-178, the contents of which are incorporated herein by reference.

Formulation of pharmaceutically acceptable excipients and carrier solutions is well-known to those of skill in the art, as is the development of suitable dosing and treatment regimens for using the particular compositions described herein in a variety of treatment regimens.

Typically, these formulations may contain at least about 0.1% of the active compound or more, although the percentage of the active ingredient(s) may, of course, be varied and may conveniently be between about 1 or 2% and about 70% or 80% or more of the weight or volume of the total formulation. Naturally, the amount of active compound in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In certain circumstances it will be desirable to deliver the rAAV-based therapeutic constructs in suitably formulated pharmaceutical compositions disclosed herein either subcutaneously, intrapancreatically, intranasally, parenterally, intravenously, intramuscularly, intrathecally, or orally, intraperitoneally, or by inhalation. In some embodiments, the administration modalities as described in U.S. Pat. Nos. 5,543,158; 5,641,515; and 5,399,363 (each specifically incorporated herein by reference in its entirety) may be used to deliver rAAVs. In some embodiments, a preferred mode of administration is by portal vein injection.

The pharmaceutical forms suitable for injectable use include sterile aqueous solutions or dispersions and sterile powders for the extemporaneous preparation of sterile injectable solutions or dispersions. Dispersions may also be prepared in glycerol, liquid polyethylene glycols, and mixtures thereof and in oils. Under ordinary conditions of storage and use, these preparations contain a preservative to prevent the growth of microorganisms. In many cases the form is sterile and fluid to the extent that easy syringability exists. It must be stable under the conditions of manufacture and storage and must be preserved against the contaminating action of microorganisms, such as bacteria and fungi. The carrier can be a solvent or dispersion medium containing, for example, water, ethanol, polyol (e.g., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For administration of an injectable aqueous solution, for example, the solution may be suitably buffered, if necessary, and the liquid diluent first rendered isotonic with sufficient saline or glucose. These particular aqueous solutions are especially suitable for intravenous, intramuscular, subcutaneous and intraperitoneal administration. In this connection, a sterile aqueous medium that can be employed will be known to those of skill in the art. For example, one dosage may be dissolved in 1 ml of isotonic NaCl solution and either added to 1000 ml of hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the host. The person responsible for administration will, in any event, determine the appropriate dose for the individual host.

Sterile injectable solutions are prepared by incorporating the active rAAV in the required amount in the appropriate solvent with various of the other ingredients enumerated herein, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. The rAAV compositions disclosed herein may also be formulated in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug-release capsules, and the like.

As used herein, “carrier” includes any and all solvents, dispersion media, vehicles, coatings, diluents, antibacterial and antifungal agents, isotonic and absorption delaying agents, buffers, carrier solutions, suspensions, colloids, and the like. The use of such media and agents for pharmaceutical active substances is well known in the art. Supplementary active ingredients can also be incorporated into the compositions. The phrase “pharmaceutically-acceptable” refers to molecular entities and compositions that do not produce an allergic or similar untoward reaction when administered to a host.

Delivery vehicles such as liposomes, nanocapsules, microparticles, microspheres, lipid particles, vesicles, and the like, may be used for the introduction of the compositions of the present disclosure into suitable host cells. In particular, the rAAV vector delivered transgenes may be formulated for delivery either encapsulated in a lipid particle, a liposome, a vesicle, a nanosphere, or a nanoparticle or the like.

Such formulations may be preferred for the introduction of pharmaceutically acceptable formulations of the nucleic acids or the rAAV constructs disclosed herein. The formation and use of liposomes is generally known to those of skill in the art. Recently, liposomes were developed with improved serum stability and circulation half-times (U.S. Pat. No. 5,741,516). Further, various methods of liposome and liposome like preparations as potential drug carriers have been described (U.S. Pat. Nos. 5,567,434; 5,552,157; 5,565,213; 5,738,868 and 5,795,587).

Liposomes have been used successfully with a number of cell types that are normally resistant to transfection by other procedures. In addition, liposomes are free of the DNA length constraints that are typical of viral-based delivery systems. Liposomes have been used effectively to introduce genes, drugs, radiotherapeutic agents, viruses, transcription factors and allosteric effectors into a variety of cultured cell lines and animals. In addition, several successful clinical trials examining the effectiveness of liposome-mediated drug delivery have been completed.

Liposomes are formed from phospholipids that are dispersed in an aqueous medium and spontaneously form multilamellar concentric bilayer vesicles (also termed multilamellar vesicles (MLVs). MLVs generally have diameters of from 25 nm to 4 μm. Sonication of MLVs results in the formation of small unilamellar vesicles (SUVs) with diameters in the range of 200 to 500 Angstroms, containing an aqueous solution in the core.

Alternatively, nanocapsule formulations of the rAAV may be used. Nanocapsules can generally entrap substances in a stable and reproducible way. To avoid side effects due to intracellular polymeric overloading, such ultrafine particles (sized around 0.1 μm) should be designed using polymers able to be degraded in vivo. Biodegradable polyalkyl-cyanoacrylate nanoparticles that meet these requirements are contemplated for use.

In addition to the methods of delivery described above, the following techniques are also contemplated as alternative methods of delivering the rAAV compositions to a host. Sonophoresis (e.g., ultrasound) has been used and described in U.S. Pat. No. 5,656,016 as a device for enhancing the rate and efficacy of drug permeation into and through the circulatory system. Other drug delivery alternatives contemplated are intraosseous injection (U.S. Pat. No. 5,779,708), microchip devices (U.S. Pat. No. 5,797,898), ophthalmic formulations (Bourlais et al., 1998), transdermal matrices (U.S. Pat. Nos. 5,770,219 and 5,783,208) and feedback-controlled delivery (U.S. Pat. No. 5,697,899).

In some embodiments, the disclosure relates to administration of one or more additional therapeutic agents to a subject who has been administered an rAAV or pharmaceutical composition as described herein.

### Kits and Related Compositions

The agents (e.g., nucleic acids, rAAV, vectors, etc.) described herein may, in some embodiments, be assembled into pharmaceutical or diagnostic or research kits to facilitate their use in therapeutic, diagnostic or research applications. A kit may include one or more containers housing the components of the disclosure and instructions for use. Specifically, such kits may include one or more agents described herein, along with instructions describing the intended application and the proper use of these agents. In certain embodiments agents in a kit may be in a pharmaceutical formulation and dosage suitable for a particular application and for a method of administration of the agents. Kits for research purposes may contain the components in appropriate concentrations or quantities for running various experiments.

In some embodiments, the disclosure relates to a kit for producing an rAAV, the kit comprising a container housing an isolated nucleic acid having a sequence of any one of SEQ ID NO: 1-8. In some embodiments, the kit further comprises instructions for producing the rAAV. In some embodiments, the kit further comprises at least one container housing an rAAV vector, wherein the rAAV vector comprises a transgene.

In some embodiments, the disclosure relates to a kit comprising a container housing an rAAV as described supra. In some embodiments, the kit further comprises a container housing a pharmaceutically acceptable carrier. For example, a kit may comprise one container housing an rAAV and a second container housing a buffer suitable for injection of the rAAV into a subject. In some embodiments, the container is a syringe.

The kit may be designed to facilitate use of the methods described herein by researchers and can take many forms. Each of the compositions of the kit, where applicable, may be provided in liquid form (e.g., in solution), or in solid form, (e.g., a dry powder). In certain cases, some of the compositions may be constitutable or otherwise processable (e.g., to an active form), for example, by the addition of a suitable solvent or other species (for example, water or a cell culture medium), which may or may not be provided with the kit. As used herein, “instructions” can define a component of instruction and/or promotion, and typically involve written instructions on or associated with packaging of the disclosure. Instructions also can include any oral or electronic instructions provided in any manner such that a user will clearly recognize that the instructions are to be associated with the kit, for example, audiovisual (e.g., videotape, DVD, etc.), Internet, and/or web-based communications, etc. The written instructions may be in a form prescribed by a governmental agency regulating the manufacture, use or sale of pharmaceuticals or biological products, which instructions can also reflects approval by the agency of manufacture, use or sale for animal administration.

The kit may contain any one or more of the components described herein in one or more containers. As an example, in one embodiment, the kit may include instructions for mixing one or more components of the kit and/or isolating and mixing a sample and applying to a subject. The kit may include a container housing agents described herein. The agents may be in the form of a liquid, gel or solid (powder). The agents may be prepared sterilely, packaged in syringe and shipped refrigerated. Alternatively, it may be housed in a vial or other container for storage. A second container may have other agents prepared sterilely. Alternatively, the kit may include the active agents premixed and shipped in a syringe, vial, tube, or other container. The kit may have one or more or all of the components required to administer the agents to an animal, such as a syringe, topical application devices, or intravenous (iv) needle tubing and bag, particularly in the case of the kits for producing specific somatic animal models.

In some cases, the methods involve transfecting cells with total cellular DNAs isolated from the tissues that potentially harbor proviral AAV genomes at very low abundance and supplementing with helper virus function (e.g., adenovirus) to trigger and/or boost AAV rep and cap gene transcription in the transfected cell. In some cases, RNA from the transfected cells provides a template for RT-PCR amplification of cDNA and the detection of novel AAVs. In cases where cells are transfected with total cellular DNAs isolated from the tissues that potentially harbor proviral AAV genomes, it is often desirable to supplement the cells with factors that promote AAV gene transcription. For example, the cells may also be infected with a helper virus, such as an Adenovirus or a Herpes Virus. In a specific embodiment, the helper functions are provided by an adenovirus. The adenovirus may be a WT adenovirus, and may be of human or non-human origin, preferably non-human primate (NHP) origin. Similarly, adenoviruses known to infect non-human animals (e.g., chimpanzees, mouse) may also be employed in the methods of the disclosure (See, e.g., U.S. Pat. No. 6,083,716). In addition to WT adenoviruses, recombinant viruses or non-viral vectors (e.g., plasmids, episomes, etc.) carrying the necessary helper functions may be utilized. Such recombinant viruses are known in the art and may be prepared according to published techniques. See, e.g., U.S. Pat. Nos. 5,871,982 and 6,251,677, which describe a hybrid Ad/AAV virus. A variety of adenovirus strains are available from the American Type Culture Collection, Manassas, Va., or available by request from a variety of commercial and institutional sources. Further, the sequences of many such strains are available from a variety of databases including, e.g., PubMed and GenBank.

Cells may also be transfected with a vector (e.g., helper vector) which provides helper functions to the AAV. The vector providing helper functions may provide adenovirus functions, including, e.g., E1a, E1b, E2a, E4ORF6. The sequences of adenovirus gene providing these functions may be obtained from any known adenovirus serotype, such as serotypes 2, 3, 4, 7, 12 and 40, and further including any of the presently identified human types known in the art. Thus, in some embodiments, the methods involve transfecting the cell with a vector expressing one or more genes necessary for AAV replication, AAV gene transcription, and/or AAV packaging.

In some cases, a novel isolated capsid gene can be used to construct and package rAAV vectors, using methods well known in the art, to determine functional characteristics associated with the novel capsid protein encoded by the gene. For example, novel isolated capsid genes can be used to construct and package rAAV vectors comprising a reporter gene (e.g., B-Galactosidase, GFP, Luciferase, etc.). The rAAV vector can then be delivered to an animal (e.g., mouse) and the tissue targeting properties of the novel isolated capsid gene can be determined by examining the expression of the reporter gene in various tissues (e.g., heart, liver, kidneys) of the animal. Other methods for characterizing the novel isolated capsid genes are disclosed herein and still others are well known in the art.

The kit may have a variety of forms, such as a blister pouch, a shrink-wrapped pouch, a vacuum sealable pouch, a sealable thermoformed tray, or a similar pouch or tray form, with the accessories loosely packed within the pouch, one or more tubes, containers, a box or a bag. The kit may be sterilized after the accessories are added, thereby allowing the individual accessories in the container to be otherwise unwrapped. The kits can be sterilized using any appropriate sterilization techniques, such as radiation sterilization, heat sterilization, or other sterilization methods known in the art. The kit may also include other components, depending on the specific application, for example, containers, cell media, salts, buffers, reagents, syringes, needles, a fabric, such as gauze, for applying or removing a disinfecting agent, disposable gloves, a support for the agents prior to administration etc.

The instructions included within the kit may involve methods for detecting a latent AAV in a cell. In addition, kits of the disclosure may include, instructions, a negative and/or positive control, containers, diluents and buffers for the sample, sample preparation tubes and a printed or electronic table of reference AAV sequence for sequence comparisons.

### Methods of Treatment

Aspects of the present disclosure provide methods for treating sialidosis and/or galactosialidosis. Additional aspects of the present disclosure provide methods for treating Alzheimer's disease.

Sialidosis is a rare neurodegenerative disease caused by deficiency of the sialic acid-cleaving enzyme Neuraminidase 1 (NEU1) and progressive buildup of sialylated glycopeptides and oligosaccharides in tissues and body fluids.

Type 1 sialidosis is the attenuated non-neuropathic form of the disease, also known as cherry red spot myoclonus syndrome. Clinical signs manifest in the second decade of life with gait abnormalities, decreased visual acuity due to bilateral macular cherry red spots, involuntary myoclonus, retina cherry red spot, ataxia, loss of visual acuity, nystagmus, seizures and hyperreflexia. It is fatal in the 3rd to 5th decade of life. Type 1 patients have abnormal ophthalmic exam, cortical and cerebellar atrophy on MRI, epileptic events on electroencephalography (EEG) and myoclonus on electromyography (EMG).

Type 2 sialidosis is a more aggressive form of the disease and is further subdivided into infantile/juvenile or congenital/hydropic forms based on the age of onset. Children with the infantile/juvenile form also develop myoclonus and seizures like the type 1 patients described above, but also develop developmental delay, milestone loss, intellectual disability and hearing and vision loss. Type 2 patients also suffer from systemic disease similar to the mucopolysaccharidoses including hepatosplenomegaly, dysostosis multiplex with joint disease, corneal clouding, facial edema and short stature. Infantile and juvenile forms of this disease are fatal in the first and second decades of life, respectively. These patients also exhibit abnormalities on EEG and MRI, as described above, and exhibit oligosacchariduria. Patients with the congenital form are stillborn or die shortly after birth with hydrops fetalis and ascites being common.

Galactosialidosis (GSL) is a lysosomal storage disease associated with a combined deficiency of beta-galactosidase and neuraminidase, secondary to a defect in protective protein/cathepsin A (PPCA). Patients have clinical manifestations typical of a lysosomal disorder, such as coarse facies, cherry red spots, vertebral changes, foam cells in the bone marrow, and vacuolated lymphocytes.

Alzheimer's Disease, a progressive form of dementia, that is characterized by degradation of amyloid precursor protein. These degraded protein products (also known as Aβ-peptides) have been shown to build up in the brain, often to toxic levels. The expression of NEU1 has been implicated in Alzheimer's Disease progression. A loss of NEU1 expression can leads to oversialylated amyloid precursor protein which can result in increased production of Aβ-peptides. It is thought that increasing NEU1 expression and/or activity may treat Alzheimer's Disease (e.g., slow progression of the disease).

Accordingly, in some embodiments, the disclosure provides isolated nucleic acids, rAAVs, compositions, and methods useful in treating sialidosis, galactosialidosis, and/or Alzheimer's Disease. The terms “treatment,” “treat,” and “treating,” as may be used interchangeably herein, refer to a clinical intervention aimed to reverse, alleviate, delay the onset of, or inhibit the progress of a, indication, disease, disorder, or one or more symptoms thereof, as described herein (e.g., sialidosis or galactosialidosis). In some embodiments, treatment may be administered after one or more symptoms have developed and/or after a disease has been diagnosed. In other embodiments, treatment may be administered in the absence of symptoms (e.g., to prevent or delay onset of a symptom or inhibit onset or progression of a disease). For example, treatment may be administered to a susceptible individual (e.g., subject) prior to the onset of symptoms (e.g., in light of a history of symptoms and/or in light of genetic or other susceptibility factors). Treatment may also be continued after symptoms have resolved, for example, to prevent or delay their recurrence.

In some aspects, the disclosure relates to a method for promoting expression of functional NEU1 protein, in a subject, the method comprising administering to the subject an effective amount of an rAAV comprising a nucleic acid engineered to express NEU1 in the subject. In some embodiments, the nucleic acid is further engineered to express CTSA. In some embodiments, the isolated nucleic acids, rAAVs, compositions, and methods are for the treatment of sialidosis and/or galactosialidosis.

In some aspects, the disclosure relates to a method for promoting expression of functional megadalton multiprotein complex in the lysosome of a target cell in a subject, the method comprising administering to the subject an effective amount of a recombinant adeno-associated viral (rAAV) vector comprising a capsid containing a nucleic acid engineered to express NEU1, wherein the megadalton multiprotein complex comprises Neuraminidase 1 (NEU1), acid beta-galactosidase (GLB1) and cathepsin A protein. In some embodiments, the nucleic acid is further engineered to express CTSA.

Methods for treating sialidosis and/or galactosialidosis in a subject may comprise administering an isolated nucleic acid, rAAV, or composition of the present disclosure that comprises a transgene encoding NEU1 and/or CTSA. In some embodiments, the method (e.g., administration of the isolated nucleic acid, rAAV, or compositions of the present disclosure) increases the expression of functional NEU1 protein in a subject. In some embodiments, the method (e.g., administration of the isolated nucleic acid, rAAV, or compositions of the present disclosure) increases the expression of functional megadalton multiprotein complex comprising Neuraminidase 1 (NEU1), acid beta-galactosidase (GLB1) and cathepsin A protein in a subject. In some embodiments, the method (e.g., administration of the isolated nucleic acid, rAAV, or compositions of the present disclosure) increases the functional expression of NEU1 protein and/or megadalton multiprotein complex by at least 10%, at least 20%, at least 30%, at least 40%, at least 50%, at least 60%, at least 70%, at least 80%, at least 90% or more, in a subject (e.g., relative to the subject prior to administration of the rAAV).

A subject may be a human, a mouse, a rat, a pig, a dog, a cat, cattle, or a non-human primate. In some embodiments, the subject is a human. Administering means contacting a cell or subject with an isolated nucleic acid, rAAV, or composition of the present disclosure. Non-limiting examples of administering including intravenous injection, intraarterial injection, intracranial injection, intrathecal injection, intracerebral injection, infusion, or inhalation.

### EXAMPLES

### Example 1. Design and Testing of rAAV Constructs

Recombinant AAV (rAAV) constructs described in this Example include one or more of the following features: a CMV enhancer, a Chicken Beta Actin (CBA) or Chicken Beta (CB) promoter, an extended CBA intron, a sequence encoding NEU1 (e.g., a sequence encoding the amino acid sequence set forth in any one of SEQ ID NOs: 5-8), and a sequence encoding CTSA (e.g., a sequence encoding the amino acid sequence set forth in SEQ ID NO: 11 or 12). The sequence encoding NEU1 or CTSA may encode a signal peptide (e.g., SEQ ID NOs: 13-18). Examples of constructs described by the disclosure are shown in FIGS. 2A-2G and in SEQ ID NOs: 21-24.

The pAAV(LysL4)-CB-Idua(SP)_Neu1 Clone 3340 construct (FIGS. 2A and 2G) comprises a 5′ ITR (R-ITR), CMV enhancer, CB promoter, the extended CBA intron, a sequence encoding an IDUA signal peptide linked to NEU1, an SV40 polyA sequence, and a 3′ ITR (L-ITR).

The pAAV(LysL4)-CB-Neu1 Clone 3341 construct (FIGS. 2B and 2E) comprises a 5′ ITR (R-ITR), CMV enhancer, CB promoter, the extended CBA intron, a sequence encoding NEU1, an SV40 polyA sequence, and a 3′ ITR (L-ITR).

The pAAV-SV40-Ctsa-BiCB6-Idua(SP)Neu1-RBG construct (FIG. 2C) is a bicistronic construct, wherein a first expression cassette comprises a CBA promoter, a sequence encoding an IDUA signal peptide linked to NEU1, an RBG polyA sequence, and an ITR; and wherein a second expression cassette comprises a CBA promoter, a sequence encoding an CTSA signal peptide linked to CTSA protein, an SV40 polyA sequence, and an ITR.

The pAAV-SV40-Ctsa-BiCB6-Neu1-RBG construct (FIGS. 2D and 2F) is a bicistronic construct, wherein a first expression cassette comprises a CBA promoter, a sequence encoding an NEU1 signal peptide linked to NEU1, an RBG polyA sequence, and an ITR; and wherein a second expression cassette comprises a CBA promoter, a sequence encoding an CTSA signal peptide linked to CTSA protein, an SV40 polyA sequence, and an ITR.

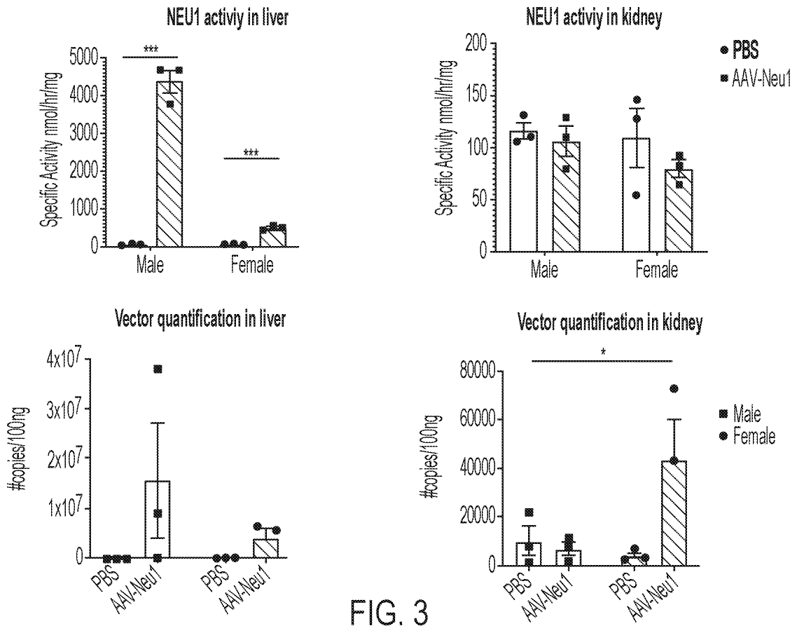

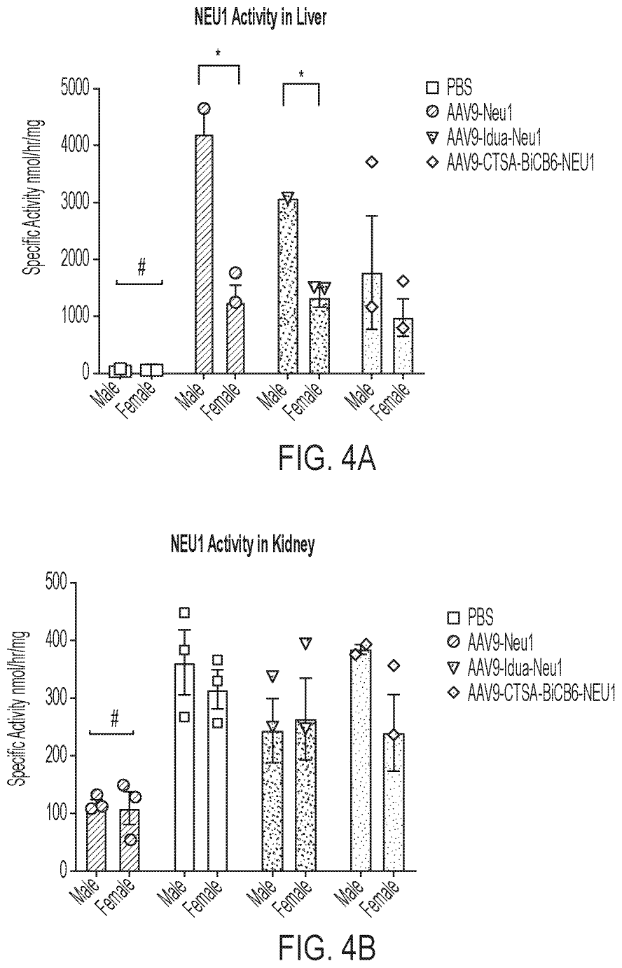

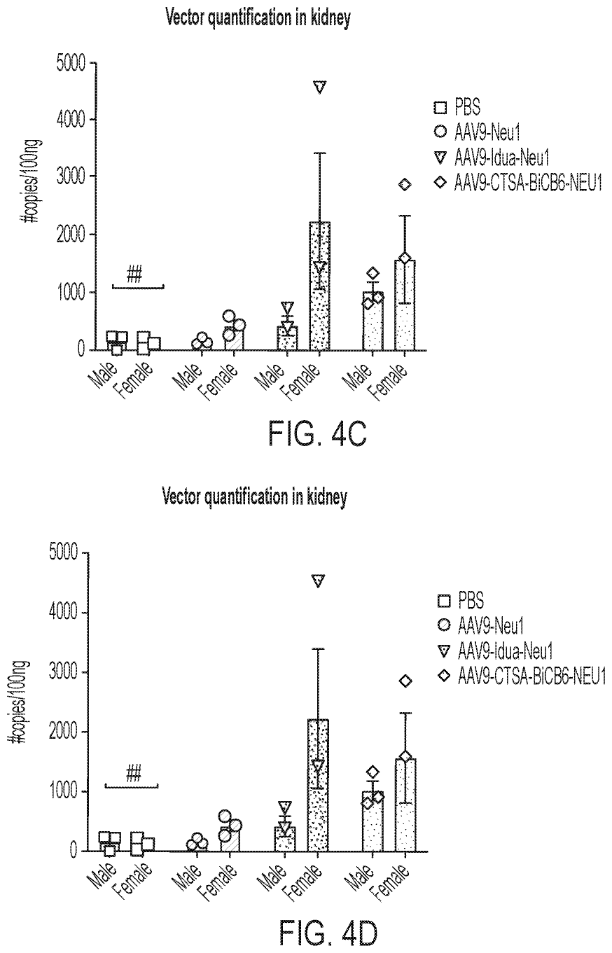

One-month-old mice were systemically injected (via hydrodynamic injection) with 1×10¹² rAAV particles comprising AAV9 capsids and one of the nucleic acid constructs described above (pAAV(LysL4)-CB-Neu1 Clone 3341, pAAV-SV40-Ctsa-BiCB6-Neu1-RBG, or pAAV(LysL4)-CB-Idua(SP)_Neu1 Clone 3340) (FIG. 3A). Mice injected with phosphate-buffered saline (PBS) were used as control. 3 male and 3 female mice were tested for each experimental condition. One month after injections, NEU1 activity and vector concentration was measured in the livers and kidneys from these mice (FIG. 3). NEU1 activity was measured by enzymatic assay, while vector concentration was measured by DNA isolation and subsequent qPCR analysis. It was observed that injection of NEU1-encoding rAAV vectors described by the disclosure resulted in increased NEU1 activity in the livers of male and female mice relative to PBS control (FIGS. 3 and 4A). There was no statistical increase observed in NEU1 activity in the kidneys of treated mice relative to PBS control (FIGS. 3 and 4B). Copies of the vector constructs were observed in both the liver and kidneys (FIGS. 3, 4C, and 4D).

These data suggest that rAAV constructs encoding NEU1 can be successfully delivered in vivo for increased expression and activity of NEU1 in the liver.

### Example 2. Sheep Model of Sialidosis

A sheep model of sialidosis was generated using CRISPR-Cas9 technology to knock out endogenous NEU1. The sheep model system is useful for evaluating the efficacy of rAAV constructs of the disclosure for treatment of sialidosis (e.g., type I sialidosis). Sheep may be systematically injected with an effective dose of rAAV construct. At varying time points following administration of the rAAV, the sheep may be evaluated for their level of symptoms associated with sialidosis. At a final time point, the sheep may be euthanized. NEU1 activity and expression in various tissues including the liver and kidneys may be tested.

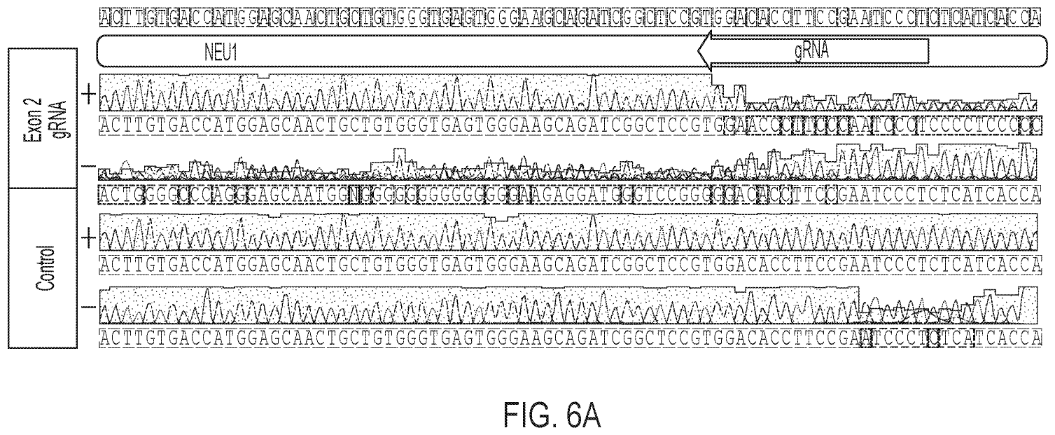

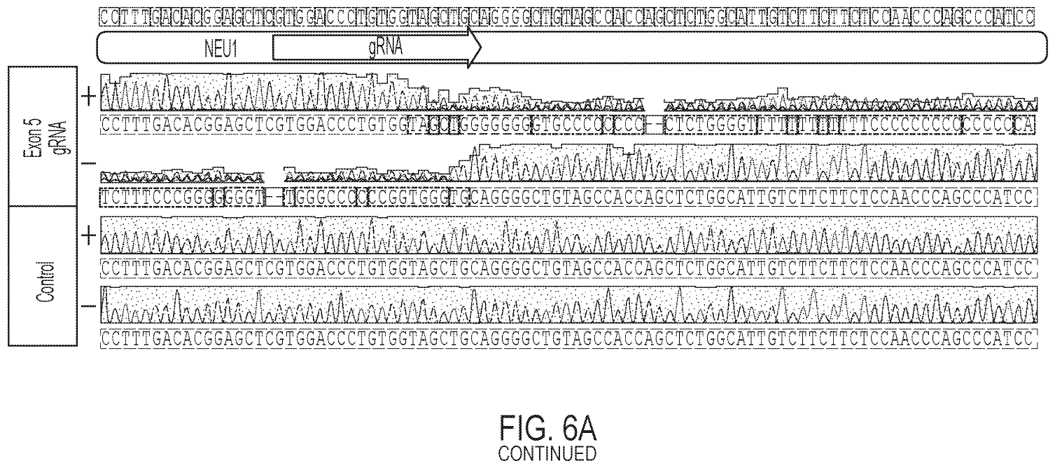

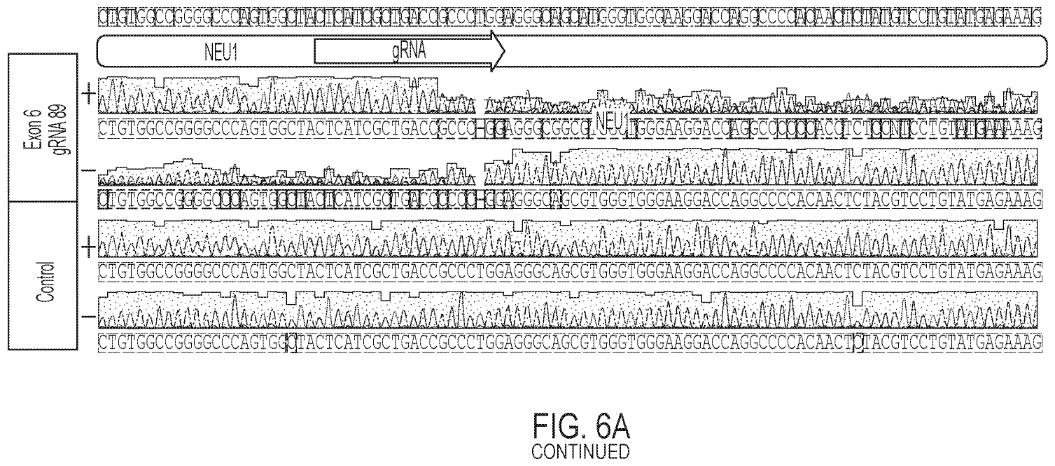

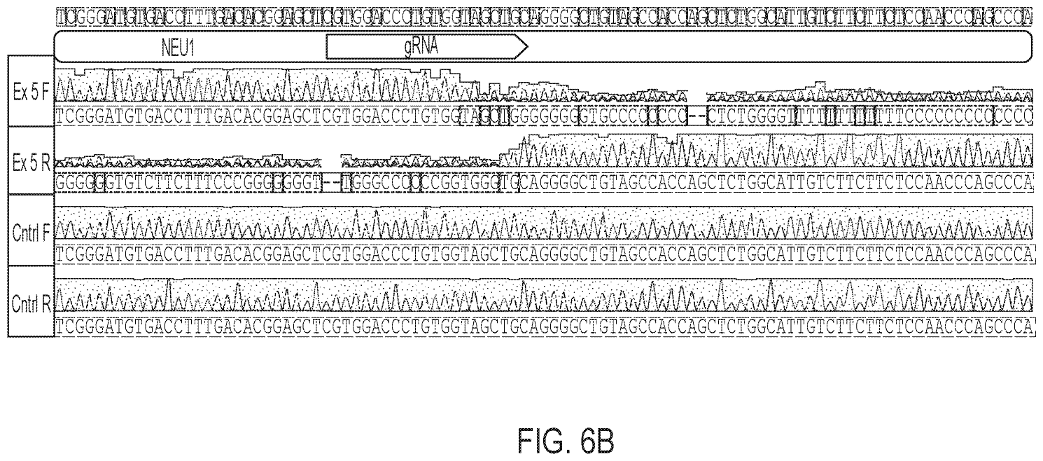

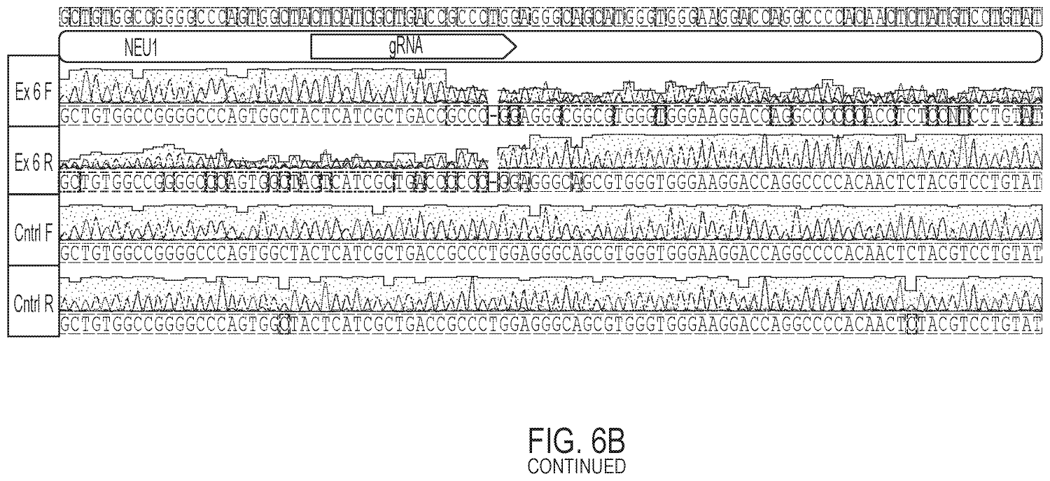

Exemplary sheep for the model system were generated from early-stage embryos that were flushed and edited by electroporation with ribonucleoproteins (RNPs) comprising Cas9 endonuclease and one or more guide RNAs targeting exons 2, 5 and 6 of NEU1. These embryos were cultured in vitro to the blastocyst stage and their DNA isolated and sequenced to confirm genomic mutations in NEU1. Embryos from the same sample were implanted into adult female sheep for gestation. The resulting offspring contained DNA comprising loss of exons 2, 5 or 6 of NEU1. FIGS. 6A-6B provide sequence maps indicating the target regions for the guide RNAs targeting exons 2, 5 and 6 of NEU1.

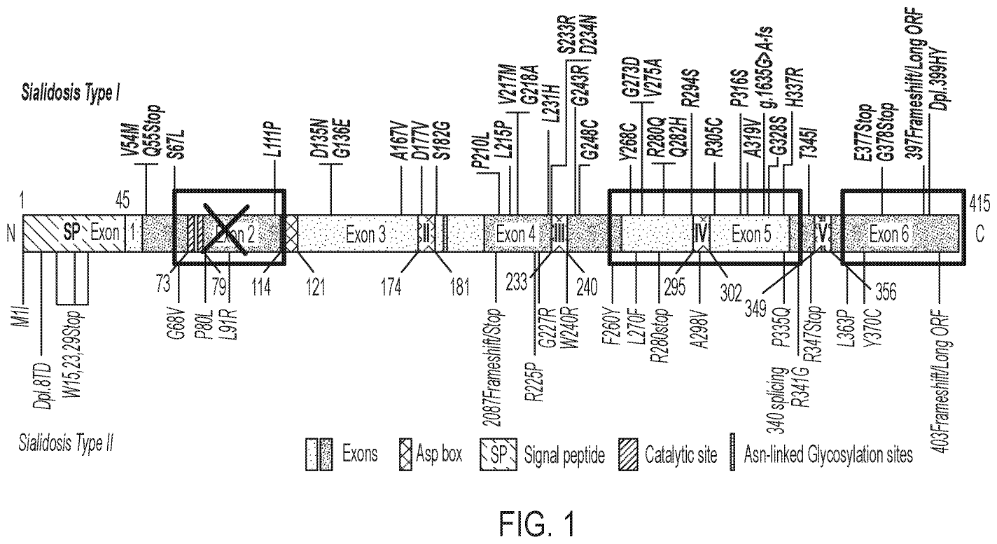

In some incidences, the NEU1 gene of the designed sheep model comprised a 3 base pair deletion in exon 5 that has been shown to be associated with type I sialidosis. Interestingly, the sheep comprising this mutation (S4 in Table 1) did not exhibit pathological phenotypes representative of sialidosis. This deletion, A320del-Neu1, is analogous to a known A319V mutation (see A319V sialidosis Type I mutation in FIG. 1). To further characterize this mutation of sheep NEU1, a construct was generated comprising a sequence encoding for A320del-Neu1. Human embryonic kidney (293) cells were transfected with this construct and a construct encoding for wild-type sheep NEU1 (WT-Neu1), and tested for NEU1 activity. Compared to non-transfected control cells, transfection of WT-Neu1 led to increased NEU1 activity; however, expression of A320del-Neu1 resulted in NEU1 activity levels comparable to the control cells. These results indicate that the A320del NEU1 variant lacks enzymatic activity.

The engineered sheep described herein exhibited phenotypes typical of patients with sialidosis. Measurements of NEU1 activity in sheep livers, kidneys, and hippocampi demonstrated high levels of knockdown in the kidney compared to control sheep, corresponding to physical abnormalities observed in the kidneys of these sheep. Furthermore, sialic acid levels were measured in sheep kidneys, livers, spleens, quadricepses, cerebellums, and spinal cords, the results of which showed differential levels of sialic acid accumulation in different organs corresponding to levels of NEU1 expression.

TABLE 1
Sialidosis sheep model animals

| | | Embryo Stage | | | | |
|---|---|---|---|---|---|---|
| | | at | CRISPR | | | Enzymatic |
| Sheep# | Sex | Electroporation | Guide | Genotype | Phenotype | Activity |
| S1 | M | 3 one-cell, | Exon 5 | Affected | Very sick, blind, wide stance, | Decreased |
| | | 2 two-cell | | mosaic: 80% | enlarged kidney, severe spine | ~50% of |
| | | | | normal | curvature, pericardial fluid, | normal |
| | | | | sequence & | died of sepsis | |
| | | | | 20% in-frame | | |
| | | | | 3-bp deletion | | |
| S2 | M | 3 one-cell, | Exon 5 | Normal | Alive, normal behavior | Normal |
| | | 2 two-cell | | sequence | | |
| S3 | M | 3 one-cell, | Exon 5 | Affected | Died in womb (autolysis) ~2 | N/A |
| | | 2 two-cell | | mosaic: | days prior to birth | |
| | | | | variety of | | |
| | | | | mutations | | |
| S4 | M | 3 one-cell, | Exon 5 | Affected | Alive, normal behavior, | Normal |
| | | 2 two-cell | | mosaic: 80% | germline mutation of 3-bp | |
| | | | | normal | deletion | |
| | | | | sequence & | | |
| | | | | 20% in-frame | | |
| | | | | 3-bp deletion | | |
| S5 | F | 3 one-cell, | Exon 5 | Affected | Died shortly after birth, | Decreased |
| | | 2 two-cell | | mosaic: 50% | enlarged kidney and liver, | 50% > of |
| | | | | frameshift | pleural effusion, retracted | normal |
| | | | | 11-bp | tendons, severe spine | |
| | | | | deletion & | curvature and cataracts | |
| | | | | 50% | | |
| | | | | frameshift 4- | | |
| | | | | bp insertion | | |
| S6 | M | 5 one-cell | Exon 5 | Normal | Alive, normal behavior | Normal |
| | | | | mosaic: | | |
| | | | | muscle | | |
| | | | | showed | | |
| | | | | variety of | | |
| | | | | mutations | | |
| S7 | F | 5 one-cell | Exon 5 | Affected | Gastroschisis, enlarged liver | Increased |
| | | | | mosaic: | and kidney, severe spine | |
| | | | | possible large | curvature, died shortly after | |
| | | | | deletions, | birth | |
| | | | | further | | |
| | | | | mutational | | |
| | | | | analysis | | |
| | | | | pending | | |
| S8 | M | 5 one-cell | Exon 5 | Affected with | Died in womb (autolysis) | N/A |
| | | | | normal | | |
| | | | | sequences | | |
| S9 | M | 5 one-cell | Exon 5 | Affected | Blind, bilateral cataracts, | Decreased |
| | | | | mosaic: 90% | nystagmus, corneal lesions, | ~15% of |
| | | | | normal | hydrocephalus, enlarged | normal |
| | | | | sequence, | ventricles, thickened dura | |
| | | | | muscle shows | and bone, thick fascia, small | |
| | | | | large deletion | liver, inflamed pancreas, | |
| | | | | | enlarged lymph node | |
| S10 | M | 5 one-cell | Exon 5 | Normal | Blind, bilateral cataracts, | Decreased |
| | | | | sequence | nystagmus, corneal lesions, | ~15% of |
| | | | | | small kidneys and liver | normal |
| S11 | M | 2 one-cell, | Exon 6 | Affected: | Small at birth, retracted | Decreased |
| | | 1 three-cell, | g89 | large deletion | forelimbs, high sodium, | 75% > of |
| | | 1 four-cell | | | enlarged kidney, | normal |
| | | | | | hydronephrosis, patent | |
| | | | | | ductus | |

### Example 3. Long-Term Study of Vector Efficiency in Sialidosis Mouse Model

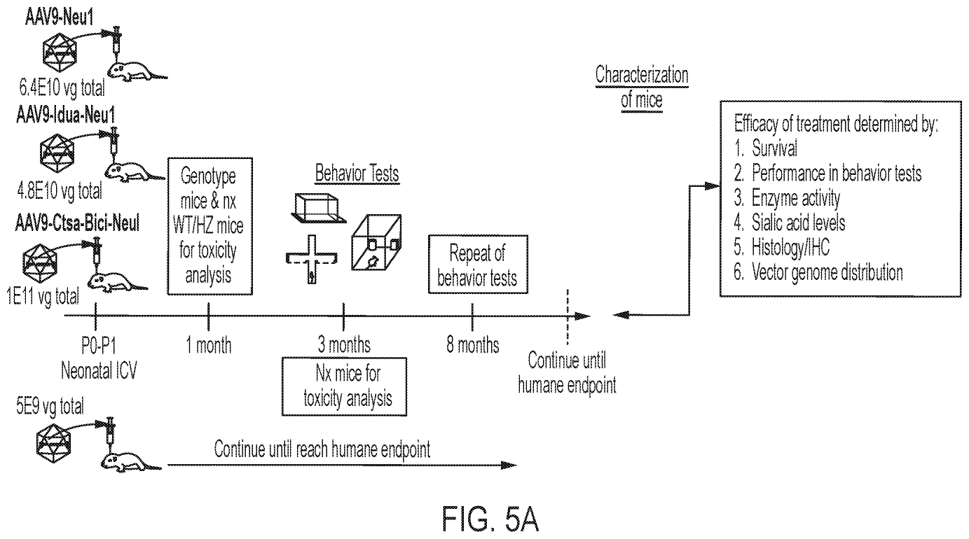

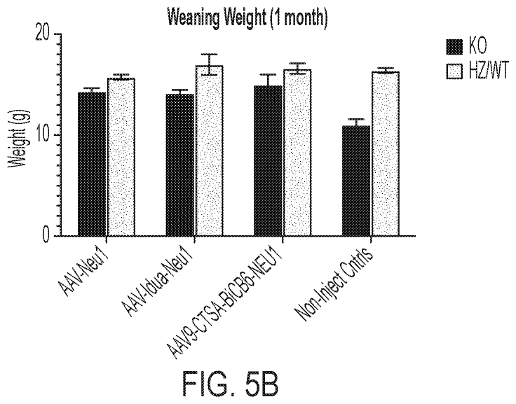

A study design for evaluation of long-term vector efficiency in a sialidosis mouse model was generated (FIG. 5A). Neonatal mice were divided into two groups based on their NEU1 genotype; Group 1=heterozygous and wild-type (HZ/WT); Group 2=knock-out (KG). The neonates from each group were intracerebroventricularly (ICV) injected on days P0-P1 after birth with one of three treatments: (1) AAV9-NEU1 rAAV particles (6.4×10¹⁰ vg total); (2) AAV9-Idua-Neu1 rAAV particles (4.8×10¹⁰ vg total); or (3) AAV9-Idua-Bicis-Neu1 rAAV particles (1×10¹¹ vg total). At one month of age, mice were weaned and weighed to test for potential physiological changes caused by the different treatment groups. In both HZ/WT and KO groups, mice treated with rAAV particles showed no difference in weight compared to non-injected control mice (FIG. 5B). Additionally at one month, nephrectomies are performed in HZ/WT mice followed by histological and immunohistochemical analyses to evaluate toxicity of NEU1-rAAV treatment. At three months of age, nephrectomies are again performed, and the resulting tissue tested for signs of toxicity. Furthermore, the 3-month-old mice are subjected to behavioral testing in order to evaluate behavioral changes induced by rAAV treatment. At 8 months of age, the behavioral tests are repeated to determine the potential longevity and/or late onset of behavioral phenotypes. The mice are maintained until they reach a humane endpoint, at which time they are evaluated for survival, behavioral test performance, liver and kidney NEU1 enzymatic activity, organ sialic acid levels, histological and immunohistochemical analyses, and vector genome distribution.

The results of this study allow for the evaluation of NEU1-rAAV vectors as a long-term treatment for sialidosis.

### Example 4. Model System for Alzheimer's Disease

It has been shown that a loss of NEU1 expression in mice leads to oversialylated amyloid precursor protein (APP), which becomes abnormally processed in lysosomes, leading to enhanced Aβ-peptide release via excessive lysosomal exocytosis. Because Aβ-peptide accumulation is correlated with Alzheimer's Disease (AD), it is possible that increasing NEU1 expression and/or activity in AD patients may ameliorate progression of this disease.

To test this hypothesis, 1-2-month-old mice are injected via ICV and thalamic injections with rAAV particles comprising AAV9 capsids and either the Ctsa-Bici-NEU1 construct, or a construct encoding for β-galactosidase flanked by ITRs. At nine months of age, the mice are subjected to behavioral testing to evaluate the effect of increased NEU1 activity on their learning and memory. The mice are then euthanized and evaluated for vector genome distribution, hippocampal NEU1 enzymatic activity, sialic acid levels, and histological and immunohistochemical analyses.

In some embodiments, young mice are transduced with rAAV encoding NEU1 or (3-galactosidase, allowed to age for 7-8 months, then evaluated for toxicity, behavior (e.g., learning and memory), NEU1 enzymatic activity, sialic acid levels, vector genome distribution, histology and immunohistochemical analyses (e.g., amyloid precursor protein and/or Aβ-peptide accumulation). In some embodiments, the mice are wild-type mice. In some embodiments, the mice are a model of Alzheimer's Disease.

The results of this study allow for the evaluation of NEU1-rAAV vectors as a treatment for AD caused by Aβ-peptide accumulation.

TABLE 2
Sequences

| SEQ | | |
|---|---|---|
| ID | | |
| NO. | Sequence* | Description |
| 1 | CTCTGTGGAGTCTAGCTGCCAGGGTCGCGGCAGCTGCGGGGA | NEU1 RNA |
| | GAGATGACTGGGGAGCGACCCAGCACGGCGCTCCCGGACAG | NM_000434.4 |
| | ACGCTGGGGGCCGCGGATTCTGGGCTTCTGGGGAGGCTGTAG | |
| | GGTTTGGGTGTTTGCCGCGATCTTCCTGCTGCTGTCTCTGGCA | |
| | GCCTCCTGGTCCAAGGCTGAGAACGACTTCGGTCTGGTGCAG | |
| | CCGCTGGTGACCATGGAGCAACTGCTGTGGGTGAGCGGGAGA | |
| | CAGATCGGCTCAGTGGACACCTTCCGCATCCCGCTCATCACA | |
| | GCCACTCCGCGGGGCACTCTTCTCGCCTTTGCTGAGGCGAGG | |
| | AAAATGTCCTCATCCGATGAGGGGGCCAAGTTCATCGCCCTG | |
| | CGGAGGTCCATGGACCAGGGCAGCACATGGTCTCCTACAGCG | |
| | TTCATTGTCAATGATGGGGATGTCCCCGATGGGCTGAACCTT | |
| | GGGGCAGTAGTGAGCGATGTTGAGACAGGAGTAGTATTTCTT | |
| | TTCTACTCCCTTTGTGCTCACAAGGCCGGCTGCCAGGTGGCCT | |
| | CTACCATGTTGGTATGGAGCAAGGATGATGGTGTTTCCTGGA | |
| | GCACACCCCGGAATCTCTCCCTGGATATTGGCACTGAAGTGT | |
| | TTGCCCCTGGACCGGGCTCTGGTATTCAGAAACAGCGGGAGC | |
| | CACGGAAGGGCCGCCTCATCGTGTGTGGCCATGGGACGCTGG | |
| | AGCGGGACGGAGTCTTCTGTCTCCTCAGCGATGATCATGGTG | |
| | CCTCCTGGCGCTACGGAAGTGGGGTCAGCGGCATCCCCTACG | |
| | GTCAGCCCAAGCAGGAAAATGATTTCAATCCTGATGAATGCC | |
| | AGCCCTATGAGCTCCCAGATGGCTCAGTCGTCATCAATGCCC | |
| | GAAACCAGAACAACTACCACTGCCACTGCCGAATTGTCCTCC | |
| | GCAGCTATGATGCCTGTGATACACTAAGGCCCCGTGATGTGA | |
| | CCTTCGACCCTGAGCTCGTGGACCCTGTGGTAGCTGCAGGAG | |
| | CTGTAGTCACCAGCTCCGGCATTGTCTTCTTCTCCAACCCAGC | |
| | ACATCCAGAGTTCCGAGTGAACCTGACCCTGCGATGGAGCTT | |
| | CAGCAATGGTACCTCATGGCGGAAAGAGACAGTCCAGCTATG | |
| | GCCAGGCCCCAGTGGCTATTCATCCCTGGCAACCCTGGAGGG | |
| | CAGCATGGATGGAGAGGAGCAGGCCCCCCAGCTCTACGTCCT | |
| | GTATGAGAAAGGCCGGAACCACTACACAGAGAGCATCTCCGT | |
| | GGCCAAAATCAGTGTCTATGGGACACTCTGAGCTGTGCCACT | |
| | GCCACAGGGGTATTCTGCCTTCAGGACTCTGCCTTCAGGAAC | |
| | ACGGGTCTGTAGAGGGTCTGCTGGAGACGCCTGAAAGACAGT | |
| | TCCATCTTCCTTTAGACTCCAGCCTTGGCAAAATCACCTTCCC | |
| | TTTACCAGGGAAATCACTTCCTTTAGGACTGAAAGCTAGGCG | |
| | TCCTCTCCCACAAAAAAGTCCTGCCCTCATCTGAGAATACTGT | |
| | CTTTCCATATGGCTAAGTGTGGCCCCACCACCCTCTCTGCCCT | |
| | CCCGGGACATTGATTGGTCCTGTCTTGGGCAGGTCTAGTGAG | |
| | CTGTAGAATTGAATCAATGTGAACTCAGGGAACTGGGGAAGG | |
| | CTGAGCCTCCTCTTTGGTGTTGCGGTAAGATAACCGACAGGG | |
| | CTGGTGAAAGTCCCCAGATGGCAGGATATTTGGTTTCAGAGT | |
| | AAGGACTAGGTGCACCACCATGACTGACTATCAATCAAAATG | |
| | TTTGTAACTTAAAATTTTTAATGAAGGATAATGAATATTTGTA | |
| | GAGTCTCTATGGTTCTGTCAATGCACATCTTCGTGTCTGTTTT | |
| | CCTCATGTATCCTTGTGAGCCTGGGTGAGTTCTGGGGAGAGA | |
| | CCTGATGTGCGTACTGCCTGTGAAAATCTGACTTTGGCAAAT | |
| | CAAATCCTCTTTTCCTTTTGACATGCCCTCTTTTTTTGTTGTTG | |
| | CTTTTTTTGAGACAGGGCTCGCTCTGTCACCCAGGCTGGAGTG | |
| | CAGTTGCACAATCACGGCTCACTGAAGCCTCAACCTCCTGGG | |
| | CTCAAGTGATCCTCACGTCTCAGCCTCCGGAGTAGTTGGGAC | |
| | TACAGGTCAGTGACACCATGCCTGGTTAATTTTTTTAATTTTT | |
| | ATTTTCAGTAGAGACAAGGTTGCGCTATGTTGCCCAGGCTGG | |
| | TATGGAACTCCTGTGCTTAAGCAATCCTCATGCCTCAGCTTCC | |
| | CAAAGTGCTGAGGTTACAGCTATGAGCCACCGCACCCAGCCT | |
| | ACATTCCTTCTTATCACCGAGAAACAGGTTGATCTTCACAGGT | |
| | GTAATGAGTATGAAGGGAGTGCCATAAGATATTTTTTATTTTT | |
| | TATTTATTTATTTTTTAATTTAATTTTTTTTTTTTTGAGATGGA | |
| | GTCTTGCTCTGGCACCCAGGCTAGAGTGCAGTGGTGCGATCT | |
| | CGGCTCACTGCAACCTCTGCCTCCCAGGTTCAAGCGATTCTTC | |
| | TGCCTCAGCTTCCCGAATAGCTGGGATTACAGGTGCCCACCA | |
| | CCACACCCGGCTAATTTTTGTATTTTTAGTAGAGACGGAGTTT | |
| | CACCATGTTGGCCAGGCTGGTCTCAAACTCCTGGCCTCAGGT | |
| | GATCCACCCGCCTCGGCCTCCCAAAGTGTTGTGATTACAAGC | |
| | ATGAGCCATGGTGCCGGCGGGCTGATTTTTTTAATTTTTAGTA | |
| | GAGACAAAGTCTCACTATATTGCCTAGGTTGGTCTCAAACTG | |
| | CTGAGCTCAAGCAATCTGCCGGCCATGTTCTCTCAAAGTGCT | |
| | GGGATTACAGGCTTGAGCCCCTGCACCCAGCCTGTAAGATAT | |
| | TTTAAAATCCACACTTGGCCAAGCGTGGTGGCTCACACATAT | |
| | AATCCCAGCACTTTGGGAGGCCAAGGTGGGCGGATCACAAGG | |
| | TCAGGAGTTCGAGACCAGCCTGGCCAATATGGTGAAATCCCA | |
| | TCTCTACTAAAAATACAAAAACTAGCCAGGCGTGTTGGCTTA | |
| | CACTTATAGTCCCAGCTACTCAGGTGGCTGAGGCAGGAGAAT | |
| | CACTTGAACCAGGAGGCGGAGTTTGCAGTGAGCTGAGATCAC | |
| | AGCACTGCACACCAGCCTCGGCAACAGAGTGAGACTCCGTCT | |
| | GAAAAAAAAAAAATCCACGCTGTTTTATATTTCTAACATGGG | |
| | GTAGGATACCATCCTCCATGAAATAGGGTGCAAGTGTGTGAA | |
| | ATGCAGATGGGAATCCTGTCCCAGCTAGCCTGACAGAGAAGT | |
| | GAGCACTTCCTATGGCAAATTATGTAAGAAAATATTCCCCAG | |
| | GCAGATGCTTATGAAAAGAATCTGTTCAGCCTGCAGGTAAGT | |
| | GGGAGAGCCCTTGCTC | |
| 2 | atggtgggagctgacccaaccaggcccagaggacctctgtcttactgggctggcaggagag | NEU1 |
| | gacagggactggccgctatcttcctgctgctggtgtctgccgctgagagcgaggcccgggc | Sequence A |
| | tgaggacgatttttccctggtgcagccactggtgacaatggagcagctgctgtgggtgagcg | |
| | gaaagcagatcggctccgtggacaccttccggatccctctgatcaccgctacaccacgcgg | |
| | cacactgctggccttcgctgaggctaggaagaagagcgccagcgacgagggcgctaagttt | |
| | atcgctatgcggcgctccaccgatcagggatctacctggagctccacagctttcatcgtgga | |
| | cgatggagaggccagcgatggactgaacctgggcgctgtggtgaacgacgtggataccgg | |
| | catcgtgtttctgatctacacactgtgcgcccacaaggtgaactgtcaggtggcttctaccatg | |
| | ctggtgtggagcaaggacgatggcatctcttggagcccccctaggaacctgtctgtggacat | |
| | cggaacagagatgttcgcccctggcccaggaagcggcatccagaagcagagggagccag | |
| | gaaagggccgcctgatcgtgtgcggacacggcaccctggagagagatggagtgttttgtct | |
| | gctgtccgacgatcacggcgcctcttggcactacggaacaggcgtgtccggaatccctttcg | |
| | gccagccaaagcacgaccacgacttcaacccagacgagtgccagccatacgagctgcctg | |
| | atggctccgtgatcatcaacgccaggaaccagaacaactaccactgccggtgtcgcatcgtg | |
| | ctgagatcttacgacgcttgtgataccctgaggcctagagacgtgacattcgatccagagctg | |
| | gtggacccagtggtggctgctggagctctggccacctctagcggcatcgtgttctttagcaac | |
| | ccagcccaccccgagttcagggtgaacctgaccctgagatggtccttttctaacggcacatc | |
| | ctggcagaaggagagggtgcaagtgtggccaggacctagcggctactcctctctgaccgcc | |
| | ctggagaactccacagacggcaagaagcagccaccccagctgtttgtgctgtacgagaagg | |
| | gactgaaccgctacaccgagagcatctccatggtgaagatcagcgtgtacggaacactgtga | |
| 3 | gaggatgacttcagcctggtgcagccgctggtgaccatggagcagctgctgtgggtgagcg | NEU1 |
| | ggaagcagatcggctctgtagacactttccgcatcccgctcatcacagccacccctcggggc | Sequence B |
| | acgctcctggccttcgctgaggccaggaaaaaatctgcatccgatgagggggccaagttcat | |
| | cgccatgaggaggtccacggaccagggtagcacgtggtcctctacagccttcatcgtagac | |
| | gatggggaggcctccgatggcctgaacctgggcgctgtggtgaacgatgtagacacaggg | |
| | atagtgttccttatctataccctctgtgctcacaaggtcaactgccaggtggcctctaccatgtt | |
| | ggtttggagtaaggacgacggcatttcctggagcccaccccggaatctctctgtggatattgg | |
| | cacagagatgtttgcccctggacctggctcaggcattcagaaacagcgggagcctgggaag | |
| | ggccggctcattgtgtgtggacacgggacgctggagcgagatggggtcttctgtctcctcag | |
| | tgatgaccacggtgcctcctggcactacggcactggagtgagcggcattccctttggccagc | |
| | ccaaacacgatcacgatttcaaccccgacgagtgccagccctacgagcttccagatggctcg | |
| | gtcatcatcaacgcccggaaccagaataactaccattgccgctgcaggatcgtcctccgcag | |
| | ctatgacgcctgtgacaccctcaggccccgggatgtgaccttcgaccctgagctcgtggacc | |
| | ctgtggtagctgcaggagcactagccaccagctccggcattgtcttcttctccaatccagccc | |
| | accctgagttccgagtgaacctgaccctgcgctggagtttcagcaatggtacatcctggcag | |
| | aaggagagggtccaggtgtggccgggacccagcggctactcgtccctgacagccctggaa | |
| | aacagcacggatggaaagaagcagcccccgcagctgttcgttctgtacgagaaaggcctga | |
| | accggtacaccgagagcatctccatggtcaaaatcagcgtctacggcacgctc | |
| 4 | gaaaacgattttggcctggtgcagccgctggtgaccatggaacagctgctgtgggtgagcg | Wild-type |
| | gccgccagattggcagcgtggatacctttcgcattccgctgattaccgcgaccccgcgcggc | human NEU1 |
| | accctgctggcgtttgcggaagcgcgcaaaatgagcagcagcgatgaaggcgcgaaattta | (without |
| | ttgcgctgcgccgcagcatggatcagggcagcacctggagcccgaccgcgtttattgtgaac | signal |
| | gatggcgatgtgccggatggcctgaacctgggcgcggtggtgagcgatgtggaaaccggc | peptide) |
| | gtggtgtttctgttttatagcctgtgcgcgcataaagcgggctgccaggtggcgagcaccatg | |
| | ctggtgtggagcaaagatgatggcgtgagctggagcaccccgcgcaacctgagcctggata | |
| | ttggcaccgaagtgtttgcgccgggcccgggcagcggcattcagaaacagcgcgaaccgc | |
| | gcaaaggccgcctgattgtgtgcggccatggcaccctggaacgcgatggcgtgttttgcctg | |
| | ctgagcgatgatcatggcgcgagctggcgctatggcagcggcgtgagcggcattccgtatg | |
| | gccagccgaaacaggaaaacgattttaacccggatgaatgccagccgtatgaactgccgga | |
| | tggcagcgtggtgattaacgcgcgcaaccagaacaactatcattgccattgccgcattgtgct | |
| | gcgcagctatgatgcgtgcgataccctgcgcccgcgcgatgtgacctttgatccggaactgg | |
| | tggatccggtggtggcggcgggcgcggtggtgaccagcagcggcattgtgttttttagcaac | |
| | ccggcgcatccggaatttcgcgtgaacctgaccctgcgctggagctttagcaacggcacca | |
| | gctggcgcaaagaaaccgtgcagctgtggccgggcccgagcggctatagcagcctggcga | |
| | ccctggaaggcagcatggatggcgaagaacaggcgccgcagctgtatgtgctgtatgaaaa | |
| | aggccgcaaccattataccgaaagcattagcgtggcgaaaattagcgtgtatggcaccctg | |
| 5 | MTGERPSTALPDRRWGPRILGFWGGCRVWVFAAIFLLLSLAAS | NEU1 protein |
| | WSKAENDFGLVQPLVTMEQLLWVSGRQIGSVDTFRIPLITATPR | NP_000425.1 |
| | GTLLAFAEARKMSSSDEGAKFIALRRSMDQGSTWSPTAFIVNDG | |
| | DVPDGLNLGAVVSDVETGVVFLFYSLCAHKAGCQVASTMLVW | |
| | SKDDGVSWSTPRNLSLDIGTEVFAPGPGSGIQKQREPRKGRLIV | |
| | CGHGTLERDGVFCLLSDDHGASWRYGSGVSGIPYGQPKQENDF | |
| | NPDECQPYELPDGSVVINARNQNNYHCHCRIVLRSYDACDTLRP | |
| | RDVTFDPELVDPVVAAGAVVTSSGIVFFSNPAHPEFRVNLTLRW | |
| | SFSNGTSWRKETVQLWPGPSGYSSLATLEGSMDGEEQAPQLYV | |
| | LYEKGRNHYTESISVAKISVYGTL | |
| 6 | MEQLLWVSGKQIGSVDTFRIPLITATPRGTLLAFAEARKKSASD | NEU1 |
| | EGAKFIAMRRSTDQGSTWSSTAFIVDDGEASDGLNLGAVVNDV | Sequence A |
| | DTGIVFLIYTLCAHKVNCQVASTMLVWSKDDGISWSPPRNLSV | |
| | DIGTEMFAPGPGSGIQKQREPGKGRLIVCGHGTLERDGVFCLLS | |
| | DDHGASWHYGTGVSGIPFGQPKHDHDFNPDECQPYELPDGSVII | |
| | NARNQNNYHCRCRIVLRSYDACDTLRPRDVTFDPELVDPVVAA | |
| | GALATSSGIVFFSNPAHPEFRVNLTLRWSFSNGTSWQKERVQV | |
| | WPGPSGYSSLTALENSTDGKKQPPQLFVLYEKGLNRYTESISMV | |
| | KISVYGTL | |
| 7 | MVGADPTRPRGPLSYWAGRRGQGLAAIFLLLVSAAESEARAED | NEU1 |
| | DFSLVQPLVTMEQLLWVSGKQIGSVDTFRIPLITATPRGTLLAFA | Sequence B |
| | EARKKSASDEGAKFIAMRRSTDQGSTWSSTAFIVDDGEASDGL | |
| | NLGAVVNDVDTGIVFLIYTLCAHKVNCQVASTMLVWSKDDGIS | |
| | WSPPRNLSVDIGTEMFAPGPGSGIQKQREPGKGRLIVCGHGTLE | |
| | RDGVFCLLSDDHGASWHYGTGVSGIPFGQPKHDHDFNPDECQP | |
| | YELPDGSVIINARNQNNYHCRCRIVLRSYDACDTLRPRDVTFDP | |
| | ELVDPVVAAGALATSSGIVFFSNPAHPEFRVNLTLRWSFSNGTS | |
| | WQKERVQVWPGPSGYSSLTALENSTDGKKQPPQLFVLYEKGLN | |
| | RYTESISMVKISVYGTL | |
| 8 | ENDFGLVQPLVTMEQLLWVSGRQIGSVDTFRIPLITATPRGTLL | Wild-type |
| | AFAEARKMSSSDEGAKFIALRRSMDQGSTWSPTAFIVNDGDVP | human NEU1 |
| | DGLNLGAVVSDVETGVVFLFYSLCAHKAGCQVASTMLVWSKD | (without |
| | DGVSWSTPRNLSLDIGTEVFAPGPGSGIQKQREPRKGRLIVCGH | signal |
| | GTLERDGVFCLLSDDHGASWRYGSGVSGIPYGQPKQENDFNPD | peptide) |
| | ECQPYELPDGSVVINARNQNNYHCHCRIVLRSYDACDTLRPRD | |
| | VTFDPELVDPVVAAGAVVTSSGIVFFSNPAHPEFRVNLTLRWSF | |
| | SNGTSWRKETVQLWPGPSGYSSLATLEGSMDGEEQAPQLYVLY | |
| | EKGRNHYTESISVAKISVYGTL | |
| 9 | agtccccgggcgcctcctggagagcaaggacgcgggggagcagagatgatccgagccgcgccgccgccgctgtt | CTSA |
| | cctgctgctgctgctgctgctgctgctagtgtcctgggcgtcccgaggcgaggcagcccccgaccaggacgaga | NM_000308.4 |
| | tccagcgcctccccgggctggccaagcagccgtctttccgccagtactccggctacctcaaaggctccggctcc | |
| | aagcacctccactactggtttgtggagtcccagaaggatcccgagaacagccctgtggtgctttggctcaatgg | |
| | gggtcccggctgcagctcactagatgggctcctcacagagcatggccccttcctggtccagccagatggtgtca | |
| | ccctggagtacaacccctattcttggaatctgattgccaatgtgttatacctggagtccccagctggggtgggc | |
| | ttctcctactccgatgacaagttttatgcaactaatgacactgaggtcgcccagagcaattttgaggcccttca | |
| | agatttcttccgcctctttccggagtacaagaacaacaaacttttcctgaccggggagagctatgctggcatct | |
| | acatccccaccctggccgtgctggtcatgcaggatcccagcatgaaccttcaggggctggctgtgggcaatgga | |
| | ctctcctcctatgagcagaatgacaactccctggtctactttgcctactaccatggccttctggggaacaggct | |
| | ttggtcttctctccagacccactgctgctctcaaaacaagtgtaacttctatgacaacaaagacctggaatgcg | |
| | tgaccaatcttcaggaagtggcccgcatcgtgggcaactctggcctcaacatctacaatctctatgccccgtgt | |
| | gctggaggggtgcccagccattttaggtatgagaaggacactgttgtggtccaggatttgggcaacatcttcac | |
| | tcgcctgccactcaagcggatgtggcatcaggcactgctgcgctcaggggataaagtgcgcatggaccccccct | |
| | gcaccaacacaacagctgcttccacctacctcaacaacccgtacgtgcggaaggccctcaacatcccggagcag | |
| | ctgccacaatgggacatgtgcaactttctggtaaacttacagtaccgccgtctctaccgaagcatgaactccca | |
| | gtatctgaagctgcttagctcacagaaataccagatcctattatataatggagatgtagacatggcctgcaatt | |
| | tcatgggggatgagtggtttgtggattccctcaaccagaagatggaggtgcagcgccggccctggttagtgaag | |
| | tacggggacagcggggagcagattgccggcttcgtgaaggagttctcccacatcgcctttctcacgatcaaggg | |
| | cgccggccacatggttcccaccgacaagcccctcgctgccttcaccatgttctcccgcttcctgaacaagcagc | |
| | catactgatgaccacagcaaccagctccacggcctgatgcagcccctcccagcctctcccgctaggagagtcct | |
| | cttctaagcaaagtgcccctgcaggccgggttctgccgccaggactgcccccttcccagagccctgtacatccc | |
| | agactgggcccagggtctcccatagacagcctgggggcaagttagcactttattcccgcagcagttcctgaatg | |
| | gggggcctggccccttctctgcttaaagaatgccctttatgatgcactgattccatcccaggaacccaacagag | |
| | ctcaggacagcccacagggaggtggtggacggactgtaattgatagattgattatggaattaaattgggtacag | |
| | cttcaaa | |
| 10 | gctccggaccaggatgaaatcgattgtctccccggcctggccaagcagccctctttccggca | CTSA |
| | atactccggctacctcagagcatcggactccaagcacttccactactggtttgtggagtcgca | proprotein |
| | gaacgacccaaagaacagccccgtggtgctttggcttaacgggggtcccggctgcagctcg | Sequence A |
| | ctcgatgggctgcttacagagcacggcccctttctgatccagccagatggtgtcaccctgga | |
| | gtacaacccctatgcttggaacctgattgccaacgtgctgtatatcgagtccccagctggggt | |
| | gggcttctcctactcggatgacaagatgtacgtgaccaatgacacagaggtggcggagaac | |
| | aattatgaagcccttaaagacttcttccgcctctttccggaatacaaggacaacaaacttttcct | |
| | gacaggagagagctatgctggcatctacatccccaccttggctgtactggtcatgcaggatc | |
| | ctagcatgaatcttcaggggctggctgtgggcaatggacttgcctcctatgagcagaacgac | |
| | aactccctggtctactttgcctactaccatggccttctggggaacagactttggacttcactgc | |
| | agacccactgctgcgctcagaacaagtgtaacttctatgacaacaaagacccagagtgtgta | |
| | aacaatctcctggaagtgtctcgaattgtgggcaaatctggcctcaacatctacaatctctatg | |
| | ctccgtgtgctggtggggtgcccggcagacatagatatgaggacacacttgtagtccaggat | |
| | tttggcaacatcttcactcgcctgccacttaagcggagatttcctgaggcactgatgcgttctg | |
| | gggacaaggtacgcttggatcctccctgcaccaacaccacagccccttccaactacctcaac | |
| | aacccctatgttcggaaggctctccacatccccgagtcgctgccccgctgggacatgtgcaa | |
| | cttcttggtgaatttacagtaccgccgcctctaccaaagcatgaactcccagtacctgaagct | |
| | gctcagttcacagaaataccagatcctgctctacaacggagatgtggacatggcctgcaactt | |
| | catgggcgatgagtggtttgtggattcgctcaaccagaagatggaggtgcagcgccggccc | |
| | tggctagtggactacggggagagcggagaacaggtagctggtttcgtgaaggagtgttcac | |
| | acatcaccttcctcaccatcaagggtgccggacacatggtccccacggacaagcctcgagct | |
| | gcttttaccatgttctcgaggttcctgaacaaagagccttac | |
| 11 | MIRAAPPPLFLLLLLLLLLVSWASRGEAAPDQDEIQRLPGLAKQ | Capthesin A |
| | PSFRQYSGYLKGSGSKHLHYWFVESQKDPENSPVVLWLNGGPG | NP_000299.3 |
| | CSSLDGLLTEHGPFLVQPDGVTLEYNPYSWNLIANVLYLESPAG | |
| | VGFSYSDDKFYATNDTEVAQSNFEALQDFFRLFPEYKNNKLFLT | |
| | GESYAGIYIPTLAVLVMQDPSMNLQGLAVGNGLSSYEQNDNSL | |
| | VYFAYYHGLLGNRLWSSLQTHCCSQNKCNFYDNKDLECVTNL | |
| | QEVARIVGNSGLNIYNLYAPCAGGVPSHFRYEKDTVVVQDLGN | |
| | IFTRLPLKRMWHQALLRSGDKVRMDPPCTNTTAASTYLNNPYV | |
| | RKALNIPEQLPQWDMCNFLVNLQYRRLYRSMNSQYLKLLSSQK | |
| | YQILLYNGDVDMACNFMGDEWFVDSLNQKMEVQRRPWLVKY | |
| | GDSGEQIAGFVKEFSHIAFLTIKGAGHMVPTDKPLAAFTMFSRF | |
| | LNKQPY | |
| 12 | APDQDEIDCLPGLAKQPSFRQYSGYLRASDSKHFHYWFVESQN | CTSA |
| | DPKNSPVVLWLNGGPGCSSLDGLLTEHGPFLIQPDGVTLEYNPY | proprotein |
| | AWNLIANVLYIESPAGVGFSYSDDKMYVTNDTEVAENNYEALK | Sequence A |
| | DFFRLFPEYKDNKLFLTGESYAGIYIPTLAVLVMQDPSMNLQGL | |
| | AVGNGLASYEQNDNSLVYFAYYHGLLGNRLWTSLQTHCCAQN | |
| | KCNFYDNKDPECVNNLLEVSRIVGKSGLNIYNLYAPCAGGVPG | |
| | RHRYEDTLVVQDFGNIFTRLPLKRRFPEALMRSGDKVRLDPPCT | |
| | NTTAPSNYLNNPYVRKALHIPESLPRWDMCNFLVNLQYRRLYQ | |
| | SMNSQYLKLLSSQKYQILLYNGDVDMACNFMGDEWFVDSLNQ | |
| | KMEVQRRPWLVDYGESGEQVAGFVKECSHITFLTIKGAGHMVP | |
| | TDKPRAAFTMFSRFLNKEPY | |
| 13 | atgaccggcgaacgcccgagcaccgcgctgccggatcgccgctggggcccgcgcattctg | NEU1 signal |
| | ggcttttggggcggctgccgcgtgtgggtgtttgcggcgatttttctgctgctgagcctggcg | peptide |
| | gcgagctggagcaaagcg | |
| 14 | atgacttccagtccaaaggcgcctcctggagagcaaggacgcaaggaagcagagatgccc | CTSA signal |
| | ggaaccgcgctgtctccactgctcttgttgctgctcctgtcctgggcgtcccggaacgaagca | peptide |
| 15 | atgcgacccccgcgtccctcctcagctatgctgacgttttttgctgcgttcttggccgcgccct | Idua signal |
| | tggcgctggct | peptide |
| 16 | MTGERPSTALPDRRWGPRILGFWGGCRVWVFAAIFLLLSLAAS | NEU1 signal |
| | WSKA | peptide |
| 17 | MTSSPKAPPGEQGRKEAEMPGTALSPLLLLLLLSWASRNEA | CTSA signal |
| | | peptide |
| 18 | MRPPRPSSAMLTFFAAFLAAPLALA | Idua signal |
| | | peptide |
| 19 | atgcggccccctaggcccagctccgccatgctgaccttctttgctgctttcctggctgctccac | Idua signal |
| | tggccctggctgaggacgatttttctctggtgcagcccctggtgacaatggagcagctgctgt | peptide + |
| | gggtgagcggaaagcagatcggctccgtggacaccttcaggatccccctgatcaccgccac | Neu1 |
| | acctagaggcacactgctggccttcgctgaggccaggaagaagagcgccagcgacgaggg | |
| | cgctaagtttatcgccatgaggagatccaccgatcagggctctacctggtctagcacagcttt | |
| | catcgtggacgatggagaggcctccgatggactgaacctgggcgctgtggtgaacgacgtg | |
| | gataccggcatcgtgtttctgatctacacactgtgcgcccacaaggtgaactgtcaggtggct | |
| | tctaccatgctggtgtggagcaaggacgatggcatctcctggtctccaccccggaacctgtct | |
| | gtggacatcggaacagagatgttcgctccaggacctggaagcggaatccagaagcagagg | |
| | gagcctggaaagggcagactgatcgtgtgcggacacggcaccctggagagggatggagtg | |
| | ttttgtctgctgtccgacgatcacggcgcctcttggcactacggaacaggcgtgagcggaat | |
| | ccccttcggccagcctaagcacgaccacgacttcaaccctgacgagtgccagccatacgag | |
| | ctgcccgatggctccgtgatcatcaacgcccggaaccagaacaactaccactgccggtgtc | |
| | gcatcgtgctgcgctcttacgacgcttgtgataccctgaggccaagagacgtgacattcgatc | |
| | ctgagctggtggacccagtggtggctgctggagctctggccacctcctctggcatcgtgttct | |
| | ttagcaaccctgcccacccagagttccgggtgaacctgaccctgcgctggagcttttccaac | |
| | ggcacatcctggcagaaggagagggtgcaagtgtggccaggaccaagcggctacagctcc | |
| | ctgaccgctctggagaactccacagacggcaagaagcagcctccacagctgtttgtgctgta | |
| | cgagaagggactgaacagatacaccgagtctatcagcatggtgaagatctccgtgtacgga | |
| | acactgtga | |
| 20 | MRPPRPSSAMLTFFAAFLAAPLALAEDDFSLVQPLVTMEQLLW | Idua signal |
| | VSGKQIGSVDTFRIPLITATPRGTLLAFAEARKKSASDEGAKFIA | peptide + |
| | MRRSTDQGSTWSSTAFIVDDGEASDGLNLGAVVNDVDTGIVFLI | Neu1 |
| | YTLCAHKVNCQVASTMLVWSKDDGISWSPPRNLSVDIGTEMFA | |
| | PGPGSGIQKQREPGKGRLIVCGHGTLERDGVFCLLSDDHGASW | |
| | HYGTGVSGIPFGQPKHDHDFNPDECQPYELPDGSVIINARNQNN | |
| | YHCRCRIVLRSYDACDTLRPRDVTFDPELVDPVVAAGALATSSG | |
| | IVFFSNPAHPEFRVNLTLRWSFSNGTSWQKERVQVWPGPSGYSS | |
| | LTALENSTDGKKQPPQLFVLYEKGLNRYTESISMVKISVYGTL | |
| 21 | cctgcaggcagctgcgcgctcgctcgctcactgaggccgcccgggcaaagcccgggcgtc | pAAV(LysL4)- |
| | gggcgacctttggtcgcccggcctcagtgagcgagcgagcgcgcagagagggagtggcc | CB- |
| | aactccatcactaggggttcctgcggccagatcttcaatattggccattagccatattattcatt | Idua(SP)_Neu1 |
| | ggttatatagcataaatcaatattggctattggccattgcatacgttgtatctatatcataatatg | Clone 3340 |
| | tacatttatattggctcatgtccaatatgaccgccatgttggcattgattattgactagttattaat | |
| | agtaatcaattacggggtcattagttcatagcccatatatggagttccgcgttacataacttacg | |
| | gtaaatggcccgcctggctgaccgcccaacgacccccgcccattgacgtcaataatgacgta | |
| | tgttcccatagtaacgccaatagggactttccattgacgtcaatgggtggagtatttacggtaa | |
| | actgcccacttggcagtacatcaagtgtatcatatgccaagtccgccccctattgacgtcaat | |
| | gacggtaaatggcccgcctggcattatgcccagtacatgaccttacgggactttcctacttgg | |
| | cagtacatctacgtattagtcatcgctattaccatggtcgaggtgagccccacgttctgcttca | |
| | ctctccccatctcccccccctccccacccccaattttgtatttatttattttttaattattttgtgca | |
| | gcgatgggggcg ggggggggggggggcgcgcgccaggcggggggggggggcga | |
| | ggggggggggggcgaggcggagaggtgggggcagccaatcagagcggcgcgctc | |
| | cgaaagtttccttttatggcgaggggggggggggccctataaaaagcgaagcgcgc | |
| | ggggggggagtcgctgcgcgctgccttcgccccgtgccccgctccgccgccgcctcgc | |
| | gccgcccgccccggctctgactgaccgcgttactcccacaggtgagcggggggacggcc | |
| | cttctcctccgggctgtaattagcgcttggtttaatgacggcttgtttcttttctgtggctgcgtg | |
| | aaagccttgaggggctccgggagggccctttgtgcggggggagcggctcggggggtgcgt | |
| | gcgtgtgtgtgtgcgtggggagcgccgcgtgcggctccgcgctgcccggcggctgtgagc | |
| | gctgcgggcgcggcgcggggctttgtgcgctccgcagtgtgcgcgaggggagcgcggcc | |
| | gggggcggtgccccgcggtgcggggggggctgcgaggggaacaaaggctgcgtgcggg | |
| | gtgtgtgcgtgggggggtgagcagggggtgtgggcgcgtcggtcgggctgcaaccccccc | |
| | tgcacccccctccccgagttgctgagcacggcccggcttcgggtgcggggctccgtacggg | |
| | gcgtggcgcggggctcgccgtgccgggcggggggggcggcaggtgggggtgccgggc | |
| | ggggggggccgcctcgggccggggagggctcggggggggggggcggcccccgg | |
| | agcgccggcggctgtcgaggcgcggcgagccgcagccattgccttttatggtaatcgtgcg | |
| | agagggcgcagggacttcctttgtcccaaatctgtgcggagccgaaatctgggaggcgccg | |
| | ccgcaccccctctagcgggcgcggggcgaagcggtgcggcgccggcaggaaggaaatgg | |
| | gcggggagggccttcgtgcgtcgccgcgccgccgtccccttctccctctccagcctcgggg | |
| | ctgtccgcggggggacggctgccttcgggggggacggggcagggggggttcggcttctg | |
| | gcgtgtgaccggcggctctagagcctctgctaaccatgttcatgccttcttctttttcctacagc | |
| | tcctgggcaacgtgctggttattgtgctgtctcatcattttggcaaagaattcgatatcaagctt | |
| | gctagccgccaccatgcggccccctaggcccagctccgccatgctgaccttctttgctgcttt | |
| | cctggctgctccactggccctggctgaggacgatttttctctggtgcagcccctggtgacaat | |
| | ggagcagctgctgtgggtgagcggaaagcagatcggctccgtggacaccttcaggatcccc | |
| | ctgatcaccgccacacctagaggcacactgctggccttcgctgaggccaggaagaagagcg | |
| | ccagcgacgagggcgctaagtttatcgccatgaggagatccaccgatcagggctctacctg | |
| | gtctagcacagctttcatcgtggacgatggagaggcctccgatggactgaacctgggcgctg | |
| | tggtgaacgacgtggataccggcatcgtgtttctgatctacacactgtgcgcccacaaggtga | |
| | actgtcaggtggcttctaccatgctggtgtggagcaaggacgatggcatctcctggtctccac | |
| | cccggaacctgtctgtggacatcggaacagagatgttcgctccaggacctggaagcggaat | |
| | ccagaagcagagggagcctggaaagggcagactgatcgtgtgcggacacggcaccctgg | |
| | agagggatggagtgttttgtctgctgtccgacgatcacggcgcctcttggcactacggaaca | |
| | ggcgtgagcggaatccccttcggccagcctaagcacgaccacgacttcaaccctgacgagt | |
| | gccagccatacgagctgcccgatggctccgtgatcatcaacgcccggaaccagaacaacta | |
| | ccactgccggtgtcgcatcgtgctgcgctcttacgacgcttgtgataccctgaggccaagag | |
| | acgtgacattcgatcctgagctggtggacccagtggtggctgctggagctctggccacctcc | |
| | tctggcatcgtgttctttagcaaccctgcccacccagagttccgggtgaacctgaccctgcgc | |
| | tggagcttttccaacggcacatcctggcagaaggagagggtgcaagtgtggccaggaccaa | |
| | gcggctacagctccctgaccgctctggagaactccacagacggcaagaagcagcctccaca | |
| | gctgtttgtgctgtacgagaagggactgaacagatacaccgagtctatcagcatggtgaaga | |
| | tctccgtgtacggaacactgtgactcgagtttttttttgcggccgcttcgagcagacatgataa | |
| | gatacattgatgagtttggacaaaccacaactagaatgcagtgaaaaaaatgctttatttgtga | |
| | aatttgtgatgctattgctttatttgtaaccattataagctgcaataaacaagttaacaacaacaa | |
| | ttgcattcattttatgtttcaggttcagggggagatgtgggaggttttttaaagcaagtaaaacc | |
| | tctacaaatgtggtaaaatcgataggccgcaggaacccctagtgatggagttggccactccc | |
| | tctctgcgcgctcgctcgctcactgaggccgggcgaccaaaggtcgcccgacgcccgggc | |
| | ggcctcagtgagcgagcgagcgcgcagctgcctgcaggacatgtgagcaaaaggccagca | |
| | aaaggccaggaaccgtaaaaaggccgcgttgctggcgtttttccataggctccgcccccctg | |
| | acgagcatcacaaaaatcgacgctcaagtcagaggtggcgaaacccgacaggactataaag | |
| | ataccaggcgtttccccctggaagctccctcgtgcgctctcctgttccgaccctgccgcttac | |
| | cggatacctgtccgcctttctcccttcgggaagcgtggcgctttctcatagctcacgctgtagg | |
| | tatctcagttcggtgtaggtcgttcgctccaagctgggctgtgtgcacgaaccccccgttcag | |
| | cccgaccgctgcgccttatccggtaactatcgtcttgagtccaacccggtaagacacgactta | |
| | tcgccactggcagcagccactggtaacaggattagcagagcgaggtatgtaggcggtgcta | |
| | cagagttcttgaagtggtggcctaactacggctacactagaagaacagtatttggtatctgcg | |
| | ctctgctgaagccagttaccttcggaaaaagagttggtagctcttgatccggcaaacaaacca | |
| | ccgctggtagcggtggtttttttgtttgcaagcagcagattacgcgcagaaaaaaaggatctc | |
| | aagaagatcctttgatcttttctacggggtctgacgctcagtggaacgaaaactcacgttaag | |
| | ggattttggtcatgagattatcaaaaaggatcttcacctagatccttttaaattaaaaatgaagtt | |
| | ttaaatcaatctaaagtatatatgagtaaacttggtctgacagttaccaatgcttaatcagtgag | |
| | gcacctatctcagcgatctgtctatttcgttcatccatagttgcctgactccccgtcgtgtagat | |
| | aactacgatacgggagggcttaccatctggccccagtgctgcaatgataccgcgagaccca | |
| | cgctcaccggctccagatttatcagcaataaaccagccagccggaagggccgagcgcaga | |
| | agtggtcctgcaactttatccgcctccatccagtctattaattgttgccgggaagctagagtaa | |
| | gtagttcgccagttaatagtttgcgcaacgttgttgccattgctacaggcatcgtggtgtcacg | |
| | ctcgtcgtttggtatggcttcattcagctccggttcccaacgatcaaggcgagttacatgatcc | |
| | cccatgttgtgcaaaaaagcggttagctccttcggtcctccgatcgttgtcagaagtaagttgg | |
| | ccgcagtgttatcactcatggttatggcagcactgcataattctcttactgtcatgccatccgta | |
| | agatgcttttctgtgactggtgagtactcaaccaagtcattctgagaatagtgtatgcggcgac | |
| | cgagttgctcttgcccggcgtcaatacgggataataccgcgccacatagcagaactttaaaa | |
| | gtgctcatcattggaaaacgttcttcggggcgaaaactctcaaggatcttaccgctgttgagat | |
| | ccagttcgatgtaacccactcgtgcacccaactgatcttcagcatcttttactttcaccagcgtt | |
| | tctgggtgagcaaaaacaggaaggcaaaatgccgcaaaaaagggaataagggcgacacg | |
| | gaaatgttgaatactcatactcttcctttttcaatattattgaagcatttatcagggttattgtctca | |
| | tgagcggatacatatttgaatgtatttagaaaaataaacaaataggggttccgcgcacatttcc | |
| | ccgaaaagtgccacctgacgtctaagaaaccattattatcatgacattaacctataaaaatag | |
| | gcgtatcacgaggccctttcgtctcgcgcgtttcggtgatgacggtgaaaacctctgacacat | |
| | gcagctcccggagacggtcacagcttgtctgtaagcggatgccgggagcagacaagcccgt | |
| | cagggcgcgtcagcgggtgttggcgggtgtcggggctggcttaactatgcggcatcagagc | |
| | agattgtactgagagtgcaccataaaattgtaaacgttaatattttgttaaaattcgcgttaaatt | |
| | tttgttaaatcagctcattttttaaccaataggccgaaatcggcaaaatcccttataaatcaaaa | |
| | gaatagcccgagatagggttgagtgttgttccagtttggaacaagagtccactattaaagaac | |
| | gtggactccaacgtcaaagggcgaaaaaccgtctatcagggcgatggcccactacgtgaac | |
| | catcacccaaatcaagttttttggggtcgaggtgccgtaaagcactaaatcggaaccctaaag | |
| | ggagcccccgatttagagcttgacggggaaagccggcgaacgtggcgagaaaggaaggg | |
| | aagaaagcgaaaggagcgggcgctaaggcgctggcaagtgtagcggtcacgctgcgcgta | |
| | accaccacacccgccgcgcttaatgcgccgctacagggcgcgtactatggttgctttgacgt | |
| | atgcggtgtgaaataccgcacagatgcgtaaggagaaaataccgcatcaggcgcc | |
| 22 | cctgcaggcagctgcgcgctcgctcgctcactgaggccgcccgggcaaagcccgggcgtc | pAAV(LysL4)- |
| | gggcgacctttggtcgcccggcctcagtgagcgagcgagcgcgcagagagggagtggcc | CB-Neu1 |
| | aactccatcactaggggttcctgcggccagatcttcaatattggccattagccatattattcatt | Clone 3341 |
| | ggttatatagcataaatcaatattggctattggccattgcatacgttgtatctatatcataatatg | |
| | tacatttatattggctcatgtccaatatgaccgccatgttggcattgattattgactagttattaat | |
| | agtaatcaattacggggtcattagttcatagcccatatatggagttccgcgttacataacttacg | |
| | gtaaatggcccgcctggctgaccgcccaacgacccccgcccattgacgtcaataatgacgta | |
| | tgttcccatagtaacgccaatagggactttccattgacgtcaatgggtggagtatttacggtaa | |
| | actgcccacttggcagtacatcaagtgtatcatatgccaagtccgccccctattgacgtcaat | |
| | gacggtaaatggcccgcctggcattatgcccagtacatgaccttacgggactttcctacttgg | |
| | cagtacatctacgtattagtcatcgctattaccatggtcgaggtgagccccacgttctgcttca | |
| | ctctccccatctcccccccctccccacccccaattttgtatttatttattttttaattattttgtgca | |
| | gcgatgggggcggggggggggggggggcgcgcgccaggcggggcggggcggggcga | |
| | ggggcggggcggggcgaggcggagaggtgcggcggcagccaatcagagcggcgcgctc | |
| | cgaaagtttccttttatggcgaggcggcggcggcggcggccctataaaaagcgaagcgcgc | |
| | ggggggggagtcgctgcgcgctgccttcgccccgtgccccgctccgccgccgcctcgc | |
| | gccgcccgccccggctctgactgaccgcgttactcccacaggtgagcggggggacggcc | |
| | cttctcctccgggctgtaattagcgcttggtttaatgacggcttgtttcttttctgtggctgcgtg | |
| | aaagccttgaggggctccgggagggccctttgtgcggggggagcggctcggggggtgcgt | |
| | gcgtgtgtgtgtgcgtggggagcgccgcgtgcggctccgcgctgcccggcggctgtgagc | |
| | gctgcgggcgcggcgcggggctttgtgcgctccgcagtgtgcgcgaggggagcgcggcc | |
| | gggggcggtgccccgcggtgcggggggggctgcgaggggaacaaaggctgcgtgcggg | |
| | gtgtgtgcgtgggggggtgagcagggggtgtgggcgcgtcggtcgggctgcaaccccccc | |
| | tgcacccccctccccgagttgctgagcacggcccggcttcgggtgcggggctccgtacggg | |
| | gcgtggcgcggggctcgccgtgccgggcggggggggcggcaggtgggggtgccgggc | |
| | ggggcggggccgcctcgggccggggagggctcggggaggggcggggcggcccccgg | |
| | agcgccggcggctgtcgaggcgcggcgagccgcagccattgccttttatggtaatcgtgcg | |
| | agagggcgcagggacttcctttgtcccaaatctgtgcggagccgaaatctgggaggcgccg | |
| | ccgcaccccctctagcgggcgcggggcgaagcggtgcggcgccggcaggaaggaaatgg | |
| | gcggggagggccttcgtgcgtcgccgcgccgccgtccccttctccctctccagcctcgggg | |
| | ctgtccgcggggggacggctgccttcgggggggacggggcagggcggggttcggcttctg | |
| | gcgtgtgaccggcggctctagagcctctgctaaccatgttcatgccttcttctttttcctacagc | |
| | tcctgggcaacgtgctggttattgtgctgtctcatcattttggcaaagaattcgatatcaagctt | |
| | gctagccgccaccatggtgggagctgacccaaccaggcccagaggacctctgtcttactgg | |
| | gctggcaggagaggacagggactggccgctatcttcctgctgctggtgtctgccgctgaga | |
| | gcgaggcccgggctgaggacgatttttccctggtgcagccactggtgacaatggagcagct | |
| | gctgtgggtgagcggaaagcagatcggctccgtggacaccttccggatccctctgatcaccg | |
| | ctacaccacgcggcacactgctggccttcgctgaggctaggaagaagagcgccagcgacg | |
| | agggcgctaagtttatcgctatgcggcgctccaccgatcagggatctacctggagctccaca | |
| | gctttcatcgtggacgatggagaggccagcgatggactgaacctgggcgctgtggtgaacg | |
| | acgtggataccggcatcgtgtttctgatctacacactgtgcgcccacaaggtgaactgtcagg | |
| | tggcttctaccatgctggtgtggagcaaggacgatggcatctcttggagcccccctaggaac | |
| | ctgtctgtggacatcggaacagagatgttcgcccctggcccaggaagcggcatccagaagc | |
| | agagggagccaggaaagggccgcctgatcgtgtgcggacacggcaccctggagagagat | |
| | ggagtgttttgtctgctgtccgacgatcacggcgcctcttggcactacggaacaggcgtgtcc | |
| | ggaatccctttcggccagccaaagcacgaccacgacttcaacccagacgagtgccagccat | |
| | acgagctgcctgatggctccgtgatcatcaacgccaggaaccagaacaactaccactgccg | |
| | gtgtcgcatcgtgctgagatcttacgacgcttgtgataccctgaggcctagagacgtgacatt | |
| | cgatccagagctggtggacccagtggtggctgctggagctctggccacctctagcggcatc | |
| | gtgttctttagcaacccagcccaccccgagttcagggtgaacctgaccctgagatggtccttt | |
| | tctaacggcacatcctggcagaaggagagggtgcaagtgtggccaggacctagcggctact | |
| | cctctctgaccgccctggagaactccacagacggcaagaagcagccaccccagctgtttgt | |
| | gctgtacgagaagggactgaaccgctacaccgagagcatctccatggtgaagatcagcgtg | |
| | tacggaacactgtgactcgagtttttttttgcggccgcttcgagcagacatgataagatacatt | |
| | gatgagtttggacaaaccacaactagaatgcagtgaaaaaaatgctttatttgtgaaatttgtg | |
| | atgctattgctttatttgtaaccattataagctgcaataaacaagttaacaacaacaattgcattc | |
| | attttatgtttcaggttcagggggagatgtgggaggttttttaaagcaagtaaaacctctacaa | |
| | atgtggtaaaatcgataggccgcaggaacccctagtgatggagttggccactccctctctgc | |
| | gcgctcgctcgctcactgaggccgggcgaccaaaggtcgcccgacgcccgggcggcctca | |
| | gtgagcgagcgagcgcgcagctgcctgcaggacatgtgagcaaaaggccagcaaaaggc | |
| | caggaaccgtaaaaaggccgcgttgctggcgtttttccataggctccgcccccctgacgagc | |
| | atcacaaaaatcgacgctcaagtcagaggtggcgaaacccgacaggactataaagatacca | |
| | ggcgtttccccctggaagctccctcgtgcgctctcctgttccgaccctgccgcttaccggata | |
| | cctgtccgcctttctcccttcgggaagcgtggcgctttctcatagctcacgctgtaggtatctc | |
| | agttcggtgtaggtcgttcgctccaagctgggctgtgtgcacgaaccccccgttcagcccga | |
| | ccgctgcgccttatccggtaactatcgtcttgagtccaacccggtaagacacgacttatcgcc | |
| | actggcagcagccactggtaacaggattagcagagcgaggtatgtaggcggtgctacagag | |
| | ttcttgaagtggtggcctaactacggctacactagaagaacagtatttggtatctgcgctctgc | |
| | tgaagccagttaccttcggaaaaagagttggtagctcttgatccggcaaacaaaccaccgct | |
| | ggtagcggtggtttttttgtttgcaagcagcagattacgcgcagaaaaaaaggatctcaagaa | |
| | gatcctttgatcttttctacggggtctgacgctcagtggaacgaaaactcacgttaagggatttt | |
| | ggtcatgagattatcaaaaaggatcttcacctagatccttttaaattaaaaatgaagttttaaatc | |
| | aatctaaagtatatatgagtaaacttggtctgacagttaccaatgcttaatcagtgaggcacct | |
| | atctcagcgatctgtctatttcgttcatccatagttgcctgactccccgtcgtgtagataactac | |
| | gatacgggagggcttaccatctggccccagtgctgcaatgataccgcgagacccacgctca | |
| | ccggctccagatttatcagcaataaaccagccagccggaagggccgagcgcagaagtggtc | |
| | ctgcaactttatccgcctccatccagtctattaattgttgccgggaagctagagtaagtagttc | |
| | gccagttaatagtttgcgcaacgttgttgccattgctacaggcatcgtggtgtcacgctcgtcg | |
| | tttggtatggcttcattcagctccggttcccaacgatcaaggcgagttacatgatcccccatgt | |
| | tgtgcaaaaaagcggttagctccttcggtcctccgatcgttgtcagaagtaagttggccgcag | |
| | tgttatcactcatggttatggcagcactgcataattctcttactgtcatgccatccgtaagatgct | |
| | tttctgtgactggtgagtactcaaccaagtcattctgagaatagtgtatgcggcgaccgagttg | |
| | ctcttgcccggcgtcaatacgggataataccgcgccacatagcagaactttaaaagtgctcat | |
| | cattggaaaacgttcttcggggcgaaaactctcaaggatcttaccgctgttgagatccagttc | |
| | gatgtaacccactcgtgcacccaactgatcttcagcatcttttactttcaccagcgtttctgggt | |
| | gagcaaaaacaggaaggcaaaatgccgcaaaaaagggaataagggcgacacggaaatgtt | |
| | gaatactcatactcttcctttttcaatattattgaagcatttatcagggttattgtctcatgagcgg | |
| | atacatatttgaatgtatttagaaaaataaacaaataggggttccgcgcacatttccccgaaaa | |
| | gtgccacctgacgtctaagaaaccattattatcatgacattaacctataaaaataggcgtatca | |
| | cgaggccctttcgtctcgcgcgtttcggtgatgacggtgaaaacctctgacacatgcagctcc | |
| | cggagacggtcacagcttgtctgtaagcggatgccgggagcagacaagcccgtcagggcg | |
| | cgtcagcgggtgttggcgggtgtcggggctggcttaactatgcggcatcagagcagattgta | |
| | ctgagagtgcaccataaaattgtaaacgttaatattttgttaaaattcgcgttaaatttttgttaaa | |
| | tcagctcattttttaaccaataggccgaaatcggcaaaatcccttataaatcaaaagaatagcc | |
| | cgagatagggttgagtgttgttccagtttggaacaagagtccactattaaagaacgtggactc | |
| | caacgtcaaagggcgaaaaaccgtctatcagggcgatggcccactacgtgaaccatcaccc | |
| | aaatcaagttttttggggtcgaggtgccgtaaagcactaaatcggaaccctaaagggagccc | |
| | ccgatttagagcttgacggggaaagccggcgaacgtggcgagaaaggaagggaagaaag | |
| | cgaaaggagcgggcgctaaggcgctggcaagtgtagcggtcacgctgcgcgtaaccacca | |
| | cacccgccgcgcttaatgcgccgctacagggcgcgtactatggttgctttgacgtatgcggt | |
| | gtgaaataccgcacagatgcgtaaggagaaaataccgcatcaggcgcc | |
| 23 | ttggccactccctctctgcgcgctcgctcgctcactgaggccgggcgaccaaaggtcgccc | pAAV-SV40- |
| | gacgcccgggctttgcccgggggcctcagtgagcgagcgagcgcgcagagagggagtg | Ctsa-BiCB6- |
| | gccaactccatcactaggggttcctagatctgaattctaccacatttgtagaggttttacttgctt | Idua(SP)Neu1- |
| | taaaaaacctcccacatctccccctgaacctgaaacataaaatgaatgcaattgttgttgttaa | RBG |
| | cttgtttattgcagcttataatggttacaaataaagcaatagcatcacaaatttcacaaataaag | |
| | catttttttcactgcattctagttgtggtttgtccaaactcatcaatgtatcttatcatgtctgtcga | |
| | gtcagtaaggctctttgttcaggaacctcgagaacatggtaaaagcagctcgaggcttgtccg | |
| | tggggaccatgtgtccggcacccttgatggtgaggaaggtgatgtgtgaacactccttcacg | |
| | aaaccagctacctgttctccgctctccccgtagtccactagccagggccggcgctgcacctc | |
| | catcttctggttgagcgaatccacaaaccactcatcgcccatgaagttgcaggccatgtccac | |
| | atctccgttgtagagcaggatctggtatttctgtgaactgagcagcttcaggtactgggagttc | |
| | atgctttggtagaggcggcggtactgtaaattcaccaagaagttgcacatgtcccagcgggg | |
| | cagcgactcggggatgtggagagccttccgaacataggggttgttgaggtagttggaaggg | |
| | gctgtggtgttggtgcagggaggatccaagcgtaccttgtccccagaacgcatcagtgcctc | |
| | aggaaatctccgcttaagtggcaggcgagtgaagatgttgccaaaatcctggactacaagtg | |
| | tgtcctcatatctatgtctgccgggcaccccaccagcacacggagcatagagattgtagatgt | |
| | tgaggccagatttgcccacaattcgagacacttccaggagattgtttacacactctgggtcttt | |
| | gttgtcatagaagttacacttgttctgagcgcagcagtgggtctgcagtgaagtccaaagtct | |
| | gttccccagaaggccatggtagtaggcaaagtagaccagggagttgtcgttctgctcatagg | |
| | aggcaagtccattgcccacagccagcccctgaagattcatgctaggatcctgcatgaccagt | |
| | acagccaaggtggggatgtagatgccagcatagctctctcctgtcaggaaaagtttgttgtcc | |
| | ttgtattccggaaagaggcggaagaagtctttaagggcttcataattgttctccgccacctctg | |
| | tgtcattggtcacgtacatcttgtcatccgagtaggagaagcccaccccagctggggactcg | |
| | atatacagcacgttggcaatcaggttccaagcataggggttgtactccagggtgacaccatct | |
| | ggctggatcagaaaggggccgtgctctgtaagcagcccatcgagcgagctgcagccggga | |
| | cccccgttaagccaaagcaccacggggctgttctttgggtcgttctgcgactccacaaacca | |
| | gtagtggaagtgcttggagtccgatgctctgaggtagccggagtattgccggaaagagggct | |
| | gcttggccaggccggggagacaatcgatttcatcctggtccggagctgcttcgttccgggac | |
| | gcccaggacaggagcagcaacaagagcagtggagacagcgcggttccgggcatctctgct | |
| | tccttgcgtccttgctctccaggaggcgcctttggactggaagtcatggtggcggctagcga | |
| | gtggacacctgtggagagaaaggcaaagtggatgtcagtaagaccaataggtgcctatcag | |
| | aaacgcaagagtcttctctgtctcgacaagcccagtttctattggtctccttaaacctgtcttgt | |
| | aaccttgatacttacctgcccagtgcctcacgaccaaactagcgctagagcttgctcccgccc | |
| | gccgcgcgcttcgctttttatagggccgccgccgccgccgcctcgccataaaaggaaacttt | |
| | cggagcgcgccgctctgattggctgccgccgcacctctccgcctcgccccgccccgcccct | |
| | cgcccccatcgctgcacaaaataattaaaaaataaataaatacaaaattgggggggggagg | |
| | ggggggagatggggagagtgaagcagaacgtggcctcggatcccccgggctgcagtatta | |
| | atagtaatcaattacggggtcattagttcatagcccatatatggagttccgcgttacataactta | |
| | cggtaaatggcccgcctggctgaccgcccaacgacccccgcccattgacgtcaataatgac | |
| | gtatgttcccatagtaacgccaatagggactttccattgacgtcaatgggtggagtatttacgg | |
| | taaactgcccacttggcagtacatcaagtgtatcatatgccaagtacgccccctattgacgtca | |
| | atgacggtaaatggcccgcctggcattatgcccagtacatgaccttatgggactttcctacttg | |
| | gcagtacatctacgtattagtcatcgctattaccatgtcgaggccacgttctgcttcactctccc | |
| | catctcccccccctccccacccccaattttgtatttatttattttttaattattttgtgcagcgatg | |
| | ggggcgaggggggggcggggcgaggcggagaggtgcggcggcagccaatcagagcg | |
| | gcgcgctccgaaagtttccttttatggcgaggggggggggggccctataaaaagcg | |
| | aagcgcgcggggggggagcaagctcgctagcactagtggcgcgcccaggtaagtttag | |
| | tctttttgtcttttatttcaggtcccggatccggtggtggtgcaaatcaaagaactgctcctcag | |
| | tggatgttgcctttacttctaggcctgtacggaagtggctagccgccaccatgcgacccccgc | |
| | gtccctcctcagctatgctgacgttttttgctgcgttcttggccgcgcccttggcgctggctga | |
| | ggatgacttcagcctggtgcagccgctggtgaccatggagcagctgctgtgggtgagcggg | |
| | aagcagatcggctctgtagacactttccgcatcccgctcatcacagccacccctcggggcac | |
| | gctcctggccttcgctgaggccaggaaaaaatctgcatccgatgagggggccaagttcatcg | |
| | ccatgaggaggtccacggaccagggtagcacgtggtcctctacagccttcatcgtagacgat | |
| | ggggaggcctccgatggcctgaacctgggcgctgtggtgaacgatgtagacacagggata | |
| | gtgttccttatctataccctctgtgctcacaaggtcaactgccaggtggcctctaccatgttggt | |
| | ttggagtaaggacgacggcatttcctggagcccaccccggaatctctctgtggatattggcac | |
| | agagatgtttgcccctggacctggctcaggcattcagaaacagcgggagcctgggaagggc | |
| | cggctcattgtgtgtggacacgggacgctggagcgagatggggtcttctgtctcctcagtgat | |
| | gaccacggtgcctcctggcactacggcactggagtgagcggcattccctttggccagccca | |
| | aacacgatcacgatttcaaccccgacgagtgccagccctacgagcttccagatggctcggtc | |
| | atcatcaacgcccggaaccagaataactaccattgccgctgcaggatcgtcctccgcagcta | |
| | tgacgcctgtgacaccctcaggccccgggatgtgaccttcgaccctgagctcgtggaccctg | |
| | tggtagctgcaggagcactagccaccagctccggcattgtcttcttctccaatccagcccacc | |
| | ctgagttccgagtgaacctgaccctgcgctggagtttcagcaatggtacatcctggcagaag | |
| | gagagggtccaggtgtggccgggacccagcggctactcgtccctgacagccctggaaaac | |
| | agcacggatggaaagaagcagcccccgcagctgttcgttctgtacgagaaaggcctgaacc | |
| | ggtacaccgagagcatctccatggtcaaaatcagcgtctacggcacgctctgactcgagcgg | |
| | ccgctctagagatctttttccctctgccaaaaattatggggacatcatgaagccccttgagcat | |
| | ctgacttctggctaataaaggaaatttattttcattgcaatagtgtgttggaattttttgtgtctct | |
| | cactcggcatgctggggagagatctaggaacccctagtgatggagttggccactccctctct | |
| | gcgcgctcgctcgctcactgaggccgcccgggcaaagcccgggcgtcgggcgacctttgg | |
| | tcgcccggcctcagtgagcgagcgagcgcgcagagagggagtggccatgcagccagctg | |
| | gcgtaatagcgaagaggcccgcaccgatcgcccttcccaacagttgcgtagcctgaatggc | |
| | gaatggcgcgacgcgccctgtagcggcgcattaagcgcggcgggtgtggtggttacgcgc | |
| | agcgtgaccgctacacttgccagcgccctagcgcccgctcctttcgctttcttcccttcctttct | |
| | cgccacgttcgccggctttccccgtcaagctctaaatcgggggctccctttagggttccgattt | |
| | agtgctttacggcacctcgaccccaaaaaacttgattagggtgatggttcacgtagtgggcca | |
| | tcgccctgatagacggtttttcgccctttgacgttggagtccacgttctttaatagtggactctt | |
| | gttccaaactggaacaacactcaaccctatctcggtctattcttttgatttataagggattttgcc | |
| | gatttcggcctattggttaaaaaatgagctgatttaacaaaaatttaacgcgaattttaacaaaa | |
| | tattaacgtttacaatttcctgatgcggtattttctccttacgcatctgtgcggtatttcacaccg | |
| | catatggtgcactctcagtacaattgctctgatgccgcatagttaagccagccccgacaccc | |
| | gccaacacccgctgacgcgccctgacgggcttgtctgctcccggcatccgcttacagacaa | |
| | gctgtgaccgtctccgggagctgcatgtgtcagaggttttcaccgtcatcaccgaaacgcgc | |
| | gagacgaaagggcctcgtgatacgcctatttttataggttaatgtcatgataataatggtttctt | |
| | agacgtcaggtggcacttttcggggaaatgtgcgcggaacccctatttgtttatttttctaaata | |
| | cattcaaatatgtatccgctcatgagacaataaccctgataaatgcttcaataatattgaaaaa | |
| | ggaagagtatgagtattcaacatttccgtgtcgcccttattcccttttttgcggcattttgccttc | |
| | ctgtttttgctcacccagaaacgctggtgaaagtaaaagatgctgaagatcagttgggtgcac | |
| | gagtgggttacatcgaactggatctcaacagcggtaagatccttgagagttttcgccccgaag | |
| | aacgttttccaatgatgagcacttttaaagttctgctatgtggcgcggtattatcccgtattgac | |
| | gccgggcaagagcaactcggtcgccgcatacactattctcagaatgacttggttgagtactc | |
| | accagtcacagaaaagcatcttacggatggcatgacagtaagagaattatgcagtgctgcca | |
| | taaccatgagtgataacactgcggccaacttacttctgacaacgatcggaggaccgaaggag | |
| | ctaaccgcttttttgcacaacatgggggatcatgtaactcgccttgatcgttgggaaccggag | |
| | ctgaatgaagccataccaaacgacgagcgtgacaccacgatgcctgtagcaatggcaacaa | |
| | cgttgcgcaaactattaactggcgaactacttactctagcttcccggcaacaattaatagactg | |
| | gatggaggcggataaagttgcaggaccacttctgcgctcggcccttccggctggctggtttat | |
| | tgctgataaatctggagccggtgagcgtgggtctcgcggtatcattgcagcactggggccag | |
| | atggtaagccctcccgtatcgtagttatctacacgacggggagtcaggcaactatggatgaa | |
| | cgaaatagacagatcgctgagataggtgcctcactgattaagcattggtaactgtcagaccaa | |
| | gtttactcatatatactttagattgatttaaaacttcatttttaatttaaaaggatctaggtgaagat | |
| | cctttttgataatctcatgaccaaaatcccttaacgtgagttttcgttccactgagcgtcagacc | |
| | ccgtagaaaagatcaaaggatcttcttgagatcctttttttctgcgcgtaatctgctgcttgcaa | |
| | acaaaaaaaccaccgctaccagcggtggtttgtttgccggatcaagagctaccaactcttttt | |
| | ccgaaggtaactggcttcagcagagcgcagataccaaatactgtccttctagtgtagccgta | |
| | gttaggccaccacttcaagaactctgtagcaccgcctacatacctcgctctgctaatcctgtta | |
| | ccagtggctgctgccagtggcgataagtcgtgtcttaccgggttggactcaagacgatagtta | |
| | ccggataaggcgcagcggtcgggctgaacggggggttcgtgcacacagcccagcttggag | |
| | cgaacgacctacaccgaactgagatacctacagcgtgagcattgagaaagcgccacgcttc | |
| | ccgaagggagaaaggcggacaggtatccggtaagcggcagggtcggaacaggagagcgc | |
| | acgagggagcttccagggggaaacgcctggtatctttatagtcctgtcgggtttcgccacctc | |
| | tgacttgagcgtcgatttttgtgatgctcgtcaggggggcggagcctatggaaaaacgccag | |
| | caacgcggcctttttacggttcctggccttttgctggccttttgctcacatgttctttcctgcgtt | |
| | atcccctgattctgtggataaccgtattaccgcctttgagtgagctgataccgctcgccgcag | |
| | ccgaacgaccgagcgcagcgagtcagtgagcgaggaagcggaagagcgcccaatacgca | |
| | aaccgcctctccccgcgcgttggccgattcattaatgcagctgggctgcaggggggggggg | |
| | ggggggggggggggggggggggggg | |
| 24 | ttggccactccctctctgcgcgctcgctcgctcactgaggccgggcgaccaaaggtcgccc | pAAV-SV40- |
| | gacgcccgggctttgcccgggcggcctcagtgagcgagcgagcgcgcagagagggagtg | Ctsa-BiCB6- |
| | gccaactccatcactaggggttcctagatctgaattctaccacatttgtagaggttttacttgctt | Neu1-RBG |
| | taaaaaacctcccacatctccccctgaacctgaaacataaaatgaatgcaattgttgttgttaa | |
| | cttgtttattgcagcttataatggttacaaataaagcaatagcatcacaaatttcacaaataaag | |
| | catttttttcactgcattctagttgtggtttgtccaaactcatcaatgtatcttatcatgtctgtcga | |
| | gtcagtaaggctctttgttcaggaacctcgagaacatggtaaaagcagctcgaggcttgtccg | |
| | tggggaccatgtgtccggcacccttgatggtgaggaaggtgatgtgtgaacactccttcacg | |
| | aaaccagctacctgttctccgctctccccgtagtccactagccagggccggcgctgcacctc | |
| | catcttctggttgagcgaatccacaaaccactcatcgcccatgaagttgcaggccatgtccac | |
| | atctccgttgtagagcaggatctggtatttctgtgaactgagcagcttcaggtactgggagttc | |
| | atgctttggtagaggcggcggtactgtaaattcaccaagaagttgcacatgtcccagcgggg | |
| | cagcgactcggggatgtggagagccttccgaacataggggttgttgaggtagttggaaggg | |
| | gctgtggtgttggtgcagggaggatccaagcgtaccttgtccccagaacgcatcagtgcctc | |
| | aggaaatctccgcttaagtggcaggcgagtgaagatgttgccaaaatcctggactacaagtg | |
| | tgtcctcatatctatgtctgccgggcaccccaccagcacacggagcatagagattgtagatgt | |
| | tgaggccagatttgcccacaattcgagacacttccaggagattgtttacacactctgggtcttt | |
| | gttgtcatagaagttacacttgttctgagcgcagcagtgggtctgcagtgaagtccaaagtct | |
| | gttccccagaaggccatggtagtaggcaaagtagaccagggagttgtcgttctgctcatagg | |
| | aggcaagtccattgcccacagccagcccctgaagattcatgctaggatcctgcatgaccagt | |
| | acagccaaggtggggatgtagatgccagcatagctctctcctgtcaggaaaagtttgttgtcc | |
| | ttgtattccggaaagaggcggaagaagtctttaagggcttcataattgttctccgccacctctg | |
| | tgtcattggtcacgtacatcttgtcatccgagtaggagaagcccaccccagctggggactcg | |
| | atatacagcacgttggcaatcaggttccaagcataggggttgtactccagggtgacaccatct | |
| | ggctggatcagaaaggggccgtgctctgtaagcagcccatcgagcgagctgcagccggga | |
| | cccccgttaagccaaagcaccacggggctgttctttgggtcgttctgcgactccacaaacca | |
| | gtagtggaagtgcttggagtccgatgctctgaggtagccggagtattgccggaaagagggct | |
| | gcttggccaggccggggagacaatcgatttcatcctggtccggagctgcttcgttccgggac | |
| | gcccaggacaggagcagcaacaagagcagtggagacagcgcggttccgggcatctctgct | |
| | tccttgcgtccttgctctccaggaggcgcctttggactggaagtcatggtggcggctagcga | |
| | gtggacacctgtggagagaaaggcaaagtggatgtcagtaagaccaataggtgcctatcag | |
| | aaacgcaagagtcttctctgtctcgacaagcccagtttctattggtctccttaaacctgtcttgt | |
| | aaccttgatacttacctgcccagtgcctcacgaccaaactagcgctagagcttgctcccgccc | |
| | gccgcgcgcttcgctttttatagggccgccgccgccgccgcctcgccataaaaggaaacttt | |
| | cggagcgcgccgctctgattggctgccgccgcacctctccgcctcgccccgccccgcccct | |
| | cgcccccatcgctgcacaaaataattaaaaaataaataaatacaaaattgggggggggagg | |
| | ggggggagatggggagagtgaagcagaacgtggcctcggatcccccgggctgcagtatta | |
| | atagtaatcaattacggggtcattagttcatagcccatatatggagttccgcgttacataactta | |
| | cggtaaatggcccgcctggctgaccgcccaacgacccccgcccattgacgtcaataatgac | |
| | gtatgttcccatagtaacgccaatagggactttccattgacgtcaatgggtggagtatttacgg | |
| | taaactgcccacttggcagtacatcaagtgtatcatatgccaagtacgccccctattgacgtca | |
| | atgacggtaaatggcccgcctggcattatgcccagtacatgaccttatgggactttcctacttg | |
| | gcagtacatctacgtattagtcatcgctattaccatgtcgaggccacgttctgcttcactctccc | |
| | catctcccccccctccccacccccaattttgtatttatttattttttaattattttgtgcagcgatg | |
| | ggggcgaggggggggcggggcgaggcggagaggtgcggcggcagccaatcagagcg | |
| | gcgcgctccgaaagtttccttttatggcgaggcggcggcggcggcggccctataaaaagcg | |
| | aagcgcgcggggggggagcaagctcgctagcactagtggcgcgcccaggtaagtttag | |
| | tctttttgtcttttatttcaggtcccggatccggtggtggtgcaaatcaaagaactgctcctcag | |
| | tggatgttgcctttacttctaggcctgtacggaagtggctagccgccaccatggtgggggca | |
| | gacccgaccagaccccggggaccgctgagctattgggcgggccgtcggggtcaggggctc | |
| | gcagcgatcttcctgctcctggtgtccgcggcggaatccgaggccagggcagaggatgact | |
| | tcagcctggtgcagccgctggtgaccatggagcagctgctgtgggtgagcgggaagcagat | |
| | cggctctgtagacactttccgcatcccgctcatcacagccacccctcggggcacgctcctgg | |
| | ccttcgctgaggccaggaaaaaatctgcatccgatgagggggccaagttcatcgccatgag | |
| | gaggtccacggaccagggtagcacgtggtcctctacagccttcatcgtagacgatggggag | |
| | gcctccgatggcctgaacctgggcgctgtggtgaacgatgtagacacagggatagtgttcct | |
| | tatctataccctctgtgctcacaaggtcaactgccaggtggcctctaccatgttggtttggagt | |
| | aaggacgacggcatttcctggagcccaccccggaatctctctgtggatattggcacagagat | |
| | gtttgcccctggacctggctcaggcattcagaaacagcgggagcctgggaagggccggctc | |
| | attgtgtgtggacacgggacgctggagcgagatggggtcttctgtctcctcagtgatgacca | |
| | cggtgcctcctggcactacggcactggagtgagcggcattccctttggccagcccaaacac | |
| | gatcacgatttcaaccccgacgagtgccagccctacgagcttccagatggctcggtcatcat | |
| | caacgcccggaaccagaataactaccattgccgctgcaggatcgtcctccgcagctatgacg | |
| | cctgtgacaccctcaggccccgggatgtgaccttcgaccctgagctcgtggaccctgtggta | |
| | gctgcaggagcactagccaccagctccggcattgtcttcttctccaatccagcccaccctga | |
| | gttccgagtgaacctgaccctgcgctggagtttcagcaatggtacatcctggcagaaggaga | |
| | gggtccaggtgtggccgggacccagcggctactcgtccctgacagccctggaaaacagca | |
| | cggatggaaagaagcagcccccgcagctgttcgttctgtacgagaaaggcctgaaccggta | |
| | caccgagagcatctccatggtcaaaatcagcgtctacggcacgctctgactcgaggcggcc | |
| | gctctagagatctttttccctctgccaaaaattatggggacatcatgaagccccttgagcatct | |
| | gacttctggctaataaaggaaatttattttcattgcaatagtgtgttggaattttttgtgtctctca | |
| | ctcggcatgctggggagagatctaggaacccctagtgatggagttggccactccctctctgc | |
| | gcgctcgctcgctcactgaggccgcccgggcaaagcccgggcgtcgggcgacctttggtc | |
| | gcccggcctcagtgagcgagcgagcgcgcagagagggagtggccatgcagccagctggc | |
| | gtaatagcgaagaggcccgcaccgatcgcccttcccaacagttgcgtagcctgaatggcga | |
| | atggcgcgacgcgccctgtagcggcgcattaagcgcggcgggtgtggtggttacgcgcag | |
| | cgtgaccgctacacttgccagcgccctagcgcccgctcctttcgctttcttcccttcctttctcg | |
| | ccacgttcgccggctttccccgtcaagctctaaatcgggggctccctttagggttccgatttag | |
| | tgctttacggcacctcgaccccaaaaaacttgattagggtgatggttcacgtagtgggccatc | |
| | gccctgatagacggtttttcgccctttgacgttggagtccacgttctttaatagtggactcttgtt | |
| | ccaaactggaacaacactcaaccctatctcggtctattcttttgatttataagggattttgccga | |
| | tttcggcctattggttaaaaaatgagctgatttaacaaaaatttaacgcgaattttaacaaaatat | |
| | taacgtttacaatttcctgatgcggtattttctccttacgcatctgtgcggtatttcacaccgcat | |
| | atggtgcactctcagtacaatctgctctgatgccgcatagttaagccagccccgacacccgc | |
| | caacacccgctgacgcgccctgacgggcttgtctgctcccggcatccgcttacagacaagct | |
| | gtgaccgtctccgggagctgcatgtgtcagaggttttcaccgtcatcaccgaaacgcgcgag | |
| | acgaaagggcctcgtgatacgcctatttttataggttaatgtcatgataataatggtttcttaga | |
| | cgtcaggtggcacttttcggggaaatgtgcgcggaacccctatttgtttatttttctaaatacat | |
| | tcaaatatgtatccgctcatgagacaataaccctgataaatgcttcaataatattgaaaaagga | |
| | agagtatgagtattcaacatttccgtgtcgcccttattcccttttttgcggcattttgccttcctgt | |
| | ttttgctcacccagaaacgctggtgaaagtaaaagatgctgaagatcagttgggtgcacgagt | |
| | gggttacatcgaactggatctcaacagcggtaagatccttgagagttttcgccccgaagaac | |
| | gttttccaatgatgagcacttttaaagttctgctatgtggcgcggtattatcccgtattgacgcc | |
| | gggcaagagcaactcggtcgccgcatacactattctcagaatgacttggttgagtactcacca | |
| | gtcacagaaaagcatcttacggatggcatgacagtaagagaattatgcagtgctgccataac | |
| | catgagtgataacactgcggccaacttacttctgacaacgatcggaggaccgaaggagctaa | |
| | ccgcttttttgcacaacatgggggatcatgtaactcgccttgatcgttgggaaccggagctga | |
| | atgaagccataccaaacgacgagcgtgacaccacgatgcctgtagcaatggcaacaacgtt | |
| | gcgcaaactattaactggcgaactacttactctagcttcccggcaacaattaatagactggat | |
| | ggaggcggataaagttgcaggaccacttctgcgctcggcccttccggctggctggtttattgc | |
| | tgataaatctggagccggtgagcgtgggtctcgcggtatcattgcagcactggggccagatg | |
| | gtaagccctcccgtatcgtagttatctacacgacggggagtcaggcaactatggatgaacga | |
| | aatagacagatcgctgagataggtgcctcactgattaagcattggtaactgtcagaccaagtt | |
| | tactcatatatactttagattgatttaaaacttcatttttaatttaaaaggatctaggtgaagatcc | |
| | tttttgataatctcatgaccaaaatcccttaacgtgagttttcgttccactgagcgtcagacccc | |
| | gtagaaaagatcaaaggatcttcttgagatcctttttttctgcgcgtaatctgctgcttgcaaac | |
| | aaaaaaaccaccgctaccagcggtggtttgtttgccggatcaagagctaccaactctttttcc | |
| | gaaggtaactggcttcagcagagcgcagataccaaatactgtccttctagtgtagccgtagtt | |
| | aggccaccacttcaagaactctgtagcaccgcctacatacctcgctctgctaatcctgttacc | |
| | agtggctgctgccagtggcgataagtcgtgtcttaccgggttggactcaagacgatagttacc | |
| | ggataaggcgcagcggtcgggctgaacggggggttcgtgcacacagcccagcttggagcg | |
| | aacgacctacaccgaactgagatacctacagcgtgagcattgagaaagcgccacgcttccc | |
| | gaagggagaaaggcggacaggtatccggtaagcggcagggtcggaacaggagagcgcac | |
| | gagggagcttccagggggaaacgcctggtatctttatagtcctgtcgggtttcgccacctctg | |
| | acttgagcgtcgatttttgtgatgctcgtcaggggggggagcctatggaaaaacgccagca | |
| | acgcggcctttttacggttcctggccttttgctggccttttgctcacatgttctttcctgcgttatc | |
| | ccctgattctgtggataaccgtattaccgcctttgagtgagctgataccgctcgccgcagccg | |
| | aacgaccgagcgcagcgagtcagtgagcgaggaagcggaagagcgcccaatacgcaaac | |
| | cgcctctccccgcgcgttggccgattcattaatgcagctgggctgcagggggggggggggg | |
| | ggggtgggggggggggggggggg | |

### Equivalents and Scope

It is to be understood that this disclosure is not limited to any or all of the particular embodiments described expressly herein, and as such may, of course, vary. It is also to be understood that the terminology used herein is for the purpose of describing particular embodiments only, and is not intended to be limiting.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are cited to disclose and describe the methods and/or materials in connection with which the publications are cited. All such publications and patents are herein incorporated by references as if each individual publication or patent were specifically and individually indicated to be incorporated by reference. Such incorporation by reference is expressly limited to the methods and/or materials described in the cited publications and patents and does not extend to any lexicographical definitions from the cited publications and patents (i.e., any lexicographical definition in the publications and patents cited that is not also expressly repeated in the disclosure should not be treated as such and should not be read as defining any terms appearing in the accompanying claims). If there is a conflict between any of the incorporated references and this disclosure, this disclosure shall control. In addition, any particular embodiment of this disclosure that falls within the prior art may be explicitly excluded from any one or more of the claims. Because such embodiments are deemed to be known to one of ordinary skill in the art, they may be excluded even if the exclusion is not set forth explicitly herein. Any particular embodiment of the disclosure can be excluded from any claim, for any reason, whether or not related to the existence of prior art. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

In the claims articles such as “a,” “an,” and “the” may mean one or more than one unless indicated to the contrary or otherwise evident from the context. Wherever used herein, a pronoun in a gender (e.g., masculine, feminine, neuter, other, etc.,) the pronoun shall be construed as gender neutral (i.e., construed to refer to all genders equally) regardless of the implied gender unless the context clearly indicates or requires otherwise. Wherever used herein, words used in the singular include the plural, and words used in the plural includes the singular, unless the context clearly indicates or requires otherwise. Claims or descriptions that include “or” between one or more members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all of the group members are present in, employed in, or otherwise relevant to a given product or process.

Furthermore, the disclosure encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists (e.g., in Markush group format), each subgroup of the elements is also disclosed, and any element(s) can be removed from the group. It should it be understood that, in general, where the disclosure, or aspects of the disclosure, is/are referred to as comprising particular elements and/or features, certain embodiments of the disclosure or aspects of the disclosure consist, or consist essentially of, such elements and/or features. For purposes of simplicity, those embodiments have not been specifically set forth in haec verba herein. It is also noted that the terms “comprising” and “containing” are intended to be open and permits the inclusion of additional elements or steps. Where ranges are given, endpoints are included in such ranges unless otherwise specified. Furthermore, unless otherwise indicated or otherwise evident from the context and understanding of one of ordinary skill in the art, values that are expressed as ranges can assume any specific value or sub-range within the stated ranges in different embodiments of the disclosure, to the tenth of the unit of the lower limit of the range, unless the context clearly dictates otherwise.

Those skilled in the art will recognize or be able to ascertain using no more than routine experimentation many equivalents to the specific embodiments described herein. The scope of the present embodiments described herein is not intended to be limited to the above Description, but rather is as set forth in the appended claims. Those of ordinary skill in the art will appreciate that various changes and modifications to this description may be made without departing from the spirit or scope of the disclosure, as defined in the following claims.

What is claimed is:

1. A recombinant adeno-associated virus (rAAV) comprising a capsid and a nucleic acid comprising a promoter and a sequence encoding NEU1.

2. The rAAV of claim 1, wherein the sequence encoding NEU1 is a mammalian sequence.

3. The rAAV of claim 2, wherein the mammalian sequence is a human, mouse, rat, goat, or sheep sequence.

4. The rAAV of any preceding claim, wherein the nucleic acid does not include a signal peptide (e.g., a native NEU1 signal peptide).

5. The rAAV of any one of claims 1-3, wherein the nucleic acid further comprises a sequence encoding a signal peptide, optionally wherein the sequence encoding a signal peptide is operably linked to the sequence encoding NEU1.

6. The rAAV of claim 5, wherein the signal peptide is a native NEU1 signal peptide or a variant thereof, optionally wherein the native NEU1 signal peptide comprises the amino acid sequence set forth in SEQ ID NO: 16.

7. The rAAV of claim 5, wherein the signal peptide is a signal peptide derived from a lysosomal protein.

8. The rAAV of claim 5 or 7, wherein the signal peptide is an iduronidase (IDUA) signal peptide, optionally wherein the IDUA signal peptide comprises the amino acid sequence set forth in SEQ ID NO: 18.

9. The rAAV of any one of claims 1-8, wherein the promoter is a chicken beta-actin (CBA) promoter, an enhanced chicken beta-actin promoter, a retroviral Rous sarcoma virus (RSV) long terminal repeat (LTR) promoter, a cytomegalovirus (CMV) promoter, a Simian vacuolating virus 40 (SV40) promoter, a dihydrofolate reductase promoter, a beta-actin promoter, a phosphoglycerol kinase (PGK) promoter, a EF1 alpha promoter, or a U6 promoter.

10. The rAAV of any one of claims 1-9, wherein the sequence encoding NEU1 comprises the nucleotide sequence set forth in any one of SEQ ID NOs: 1-4.

11. The rAAV of any one of claims 1-10, wherein the sequence encoding NEU1 encodes a NEU1 protein comprising an amino acid sequence as set forth in any one of SEQ ID NOs: 5-8.

12. The rAAV of any one of claims 1-11, wherein the sequence encoding NEU1 is a codon-optimized human NEU1 sequence.

13. The rAAV of any one of claims 1-12, wherein the nucleic acid further comprises a sequence encoding cathepsin A.

14. The rAAV of claim 13, wherein the nucleic acid comprises a first expression cassette engineered to express NEU1 and a second expression cassette engineered to express cathepsin A.

15. The rAAV of claim 13 or 14, wherein the sequence encoding cathepsin A comprises the nucleotide sequence set forth in any one of SEQ ID NOs: 9-10.

16. The rAAV of any one of claims 13-15, wherein the sequence encoding cathepsin A encodes a cathepsin A protein comprising the amino acid sequence set forth in any one of SEQ ID NOs: 11-12.

17. The rAAV of any one of claims 1-16, wherein the nucleic acid further comprises one or more enhancer sequences, optionally a cytomegalovirus (CMV) enhancer sequence and/or an SV40 enhancer sequence.

18. The rAAV of any one of claims 1-17, wherein the nucleic acid comprises one or more ITRs, wherein each ITR is selected from the group consisting of AAV1 ITR, AAV2 ITR, AAV3 ITR, AAV4 ITR, AAV5 ITR, and AAV6 ITR.

19. The rAAV of any one of claims 1-18, wherein the nucleic acid comprises the nucleotide sequence set forth in any one of SEQ ID NOs: 21-24.

20. The rAAV of any one of claims 1-19, wherein the capsid is an AAV1, AAV2, AAV3, AAV4, AAV5, AAV6, AAV7, AAV8, or AAV9 capsid protein.

21. A pharmaceutical composition comprising (i) the rAAV of any one of claims 1-20; and (ii) a pharmaceutically acceptable excipient or solution.

22. An isolated nucleic acid comprising the sequence as set forth in any one of SEQ ID NOs: 21-24.

23. A host cell comprising the rAAV of any one of claims 1-20 or the isolated nucleic acid construct of claim 22.

24. The host cell of claim 23, wherein the cell is a mammalian cell, optionally a human cell, bacterial cell, yeast cell, or insect cell.

25. A method for promoting expression of neuraminidase 1 (NEU1) protein in a subject, the method comprising administering to the subject the rAAV of any one of claims 1-20, the pharmaceutical composition of claim 21, the isolated nucleic acid of claim 22, or the host cell of claim 23 or 24.

26. A method for promoting expression of multiprotein complex in the lysosome of a target cell in a subject, the method comprising administering to the subject the rAAV of any one of claims 1-20, the pharmaceutical composition of claim 21, the isolated nucleic acid of claim 22, or the host cell of claim 23 or 24, wherein the multiprotein complex comprises Neuraminidase 1 (NEU1), acid beta-galactosidase (GLB1) and cathepsin A protein.

27. A method for treating sialidosis or galactosialidosis in a subject in need thereof, the method comprising administering to the subject the rAAV of any one of claims 1-20, the pharmaceutical composition of claim 21, the isolated nucleic acid of claim 22, or the host cell of claim 23 or 24.

28. The method of any one of claims 25-27, wherein the subject has Type 1 sialidosis or Type 2 sialidosis.

29. The method of any one of claims 25-28, wherein the subject has one or more mutations in an endogenous NEU1 gene and/or endogenous CTSA gene, optionally wherein the subject has a deletion in exon 2, exon 5, exon 6, and/or a A319V substitution.

30. A method for treating Alzheimer's Disease in a subject in need thereof, the method comprising administering to the subject the rAAV of any one of claims 1-20, the pharmaceutical composition of claim 21, the isolated nucleic acid of claim 22, or the host cell of claim 23 or 24.

31. The method of any one of claims 25-30, wherein administration of the rAAV vector is by systemic injection.
